(19) United States
(12) Patent Application Publication
(10) Pub. No.: US 2010/0279354 A1
(43) Pub. Date: Nov. 4, 2010

(54) ADAPTING MICROORGANISMS FOR AGRICULTURAL PRODUCTS

(75) Inventor: Eudes de Crecy, Gainesville, FL (US)

Correspondence Address: WILSON, SONSINI, GOODRICH & ROSATI, 650 PAGE MILL ROAD, PALO ALTO, CA, 94304-1050, US

(73) Assignee: EVOLUGATE, LLC, Gainesville, FL (US)

(21) Appl. No.: 12/770,656

(22) Filed: Apr. 29, 2010

Related U.S. Application Data
(60) Provisional application No. 61/173,934, filed on Apr. 29, 2009.
(60) Provisional application No. 61/310,855, filed on Mar. 5, 2010.

(51) Int. Cl. C12P 21/00; C12N 1/00; C12N 1/20; C12N 1/16; C12N 1/12; C12N 1/14; C12P 1/00; C12M 1/00

(52) U.S. Cl. 435/71.1; 435/243; 435/252.1; 435/255.1; 435/257.1; 435/254.1; 435/257.3; 435/41; 435/170; 435/171; 435/289.1

(57) ABSTRACT

Disclosed herein are methods and compositions to convert a carbonaceous material to produce a fatty acid, biofuel, biodiesel, or other useful end-product. Organisms are evolutionarily modified to utilize carbon and/or nitrogen sources available in a carbonaceous material. Culture of the evolutionarily modified organism on the carbonaceous material in turn produces a fatty acid, biofuel, biodiesel, or other useful end-product by a process of conversion performed by evolutionarily modified organisms.

### CROSS-REFERENCE

This application claims the benefit of U.S. Provisional Application No. 61/173,934, filed Apr. 29, 2009, and No. 61/310,855, filed Mar. 5, 2010, which applications are incorporated herein by reference.

### BACKGROUND

Petroleum is a non-renewable resource. As a result, there are concerns about the eventual depletion of petroleum reserves. Unlike petroleum, biologically derived hydrocarbon products are renewable hydrocarbon products.

Biofuel is a fuel derived from biological sources. For example, biologically produced lipids such as biomass oils derived from plants, algae, and animal fats can be used for biofuel (Johnson D, 1987, Overview of the DOE/SERI aquatic species program FY 1986 Solar Energy Institute, Colorado, incorporated herein by reference). Biomass oils can be used as fuels in a variety of ways: directly as boiler fuels, processed into biodiesel (methyl esters), or processed into “biodistallates” via refinery technology (Tyson et al, Biomass Oil Analysis: Research Needs and Recommendations, National Renewable Energy Laboratory, June 2004, incorporated herein by reference).

Producing biofuel from microorganisms can be an efficient way to produce biodiesel and other hydrocarbon based products. Biofuel obtained from microorganisms can also be non-toxic, biodegradable and free of sulfur. As most of the carbon dioxide typically released from burning biofuel is recycled from what was absorbed during the growth of the microorganisms (e.g., algae and bacteria), it is believed that the burning of biofuel releases less carbon dioxide than from the burning of petroleum, which releases carbon dioxide from a source that has been previously stored within the earth for centuries. Thus, utilizing microorganisms for the production of biofuel can result in lower greenhouse gases such as carbon dioxide.

Some species of microorganisms can be ideally suited for fatty acid or biofuel production due to their high oil content. Certain microorganisms contain lipids and/or other desirable hydrocarbon compounds as membrane components, storage products, metabolites and sources of energy. The percentages in which the lipids, hydrocarbon compounds and fatty acids are expressed in the microorganism will vary depending on the type of microorganism that is grown.

Today's biofuel industry produces a large amount of biomass as byproducts. For example, corn distillers produce a large amount of stillage in the course of producing ethanol from corn. Regardless of the source of stillage, whether it is from corn or other types of grains such as sorghum grains, stillage in general has a complex composition. However, stillage commonly includes non-fermented fibers (e.g., from the hull and tipcap of the corn kernel, particles of the corn germ with high oil content, oil and other lipids, gluten from the non-fermented portions of the corn kernel, any residual unreacted starch, solubles such as proteins and enzymes, and/or the byproducts and residue of fermentation including dead yeast cells). The particle sizes can range widely from broken parts, such as for kernels of 1-2 millimeters in size, down to fines in the under 10 micron range. Typically, stillage is dewatered to produce animal feeds. For example, corn stillage can be converted to produce animal feeds. Stillages such as wet distillers grains with solubles (WDGS); dry distillers grains with solubles (DDGS); condensed distillers grains with solubles (CDS); and distillers grains (DG), can be produced, and used by to produce a biofuel or other valuable chemical product.

A large amount of agricultural byproducts are produced in modern agricultural operation. For example, in fruit and vegetable processing plants, a large amount of peels are produced. In oil-extracting processes, seeds or fruits or their shells are produced as a waste after oil extraction. In the processing of sugarcane or sorghum, large amounts of bagasses are produced during the extraction process. These agricultural products are useful for producing biofuels and other chemical products. Provided herein are methods and compositions for culturing an evolutionarily modified organsim (EMO) to use carbonaceous materials, such as an agricultural products to produce useful end products. The EMOs can thereby produce biofuels or other useful end products

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In one aspect, provided herein is an evolutionarily modified organism wherein said evolutionarily modified organism's growth rate or end product yield during culture with a carbonaceous material is greater than the growth rate or end product yield of a non-evolutionarily modified organism, wherein said greater growth rate or product yield is due to a non-genetically engineered modification. In one embodiment the organism is a bacterium, yeast, alga, or fungus. In another embodiment the organism is Chlorella protothecoides.

In another embodiment the carbonaceous material is a stillage, bagasse, stover, peel, seed cake, seed, sugar beet, a wood chip, or any combination thereof. In another embodiment the stillage is wet distillers grains with solubles (WDGS), dry distillers grains with solubles (DDGS), condensed distillers grains with solubles (CDS), or distillers grains (DG). In another embodiment the organism has a greater end product yield of a non-evolutionarily modified organism. In another embodiment the end product is a fatty acid.

In another aspect, provided herein is an evolutionarily modified organism wherein said evolutionarily modified organism's growth rate or end product yield during culture with a carbonaceous material is greater than the growth rate or end product yield of a non-evolutionarily modified organism, wherein said evolutionarily modified organism is modified by continuous culture inside a flexible sterile tube filled with growth medium.

In another aspect, provided herein is a method of producing an evolutionarily evolved organism comprising: exposing an organism to a carbonaceous material; continuously culturing said organism; and producing an evolutionarily evolved organism from said organism, when said evolutionarily evolved organism has a growth rate or end product yield on said carbonaceous material that is greater than the growth rate or end product yield observed for said organism not continuously cultured with said carbonaceous material.

In one embodiment the organism is a bacterium, yeast, alga, or fungus. In another embodiment the organism is Chlorella protothecoides. In another embodiment the carbonaceous material is a stillage, bagasse, stover, peel, seed cake, seed, sugar beet, a wood chip, or any combination thereof. In another embodiment the stillage is wet distillers grains with solubles (WDGS), dry distillers grains with solubles (DDGS), condensed distillers grains with solubles (CDS), or distillers grains (DG). In another embodiment the stillage is hydrolyzed stillage. In another embodiment the hydrolysis is enzymatic hydrolysis with alpha-amylase. In another embodiment the stillage is corn stillage. In another embodiment the organism is placed in a flexible tubing, wherein said tubing is subdivided by an operation of a gate into one or more discreet chambers. In another embodiment the organism is a naturally occurring organism. In another embodiment the organism is a genetically modified organism.

In another aspect, provided herein is a method of producing an end product comprising: contacting an evolutionarily evolved organism with a carbonaceous material culturing said carbonaceous material with said organism; and separating an end product from said culture of. In one embodiment the organism is a bacterium, yeast, alga, or fungus. In another embodiment the organism is Chlorella protothecoides. In another embodiment the carbonaceous material is a stillage, bagasse, stover, peel, seed cake, seed, sugar beet, a wood chip, or any combination thereof. In another embodiment the stillage is wet distillers grains with solubles (WDGS), dry distillers grains with solubles (DDGS), condensed distillers grains with solubles (CDS), or distillers grains (DG). In another embodiment the stillage is hydrolyzed stillage. In another embodiment the hydrolysis is enzymatic hydrolysis with alpha-amylase. In another embodiment the stillage is corn stillage. In another embodiment the method further comprises extracting oil from said end product. In another embodiment the method further comprises processing said oil to a biofuel. In another embodiment the biofuel is a biodiesel. In another embodiment the end product is a protein. In another embodiment the protein is an algal protein. In another embodiment the end product is a vitamin.

In another aspect, provided herein is a biofuel manufacturing plant, comprising: a culture unit comprising an evolutionary modified organism and a carbonaceous material wherein said carbonaceous material is said evolutionary modified organism's primary carbon source; a processing unit for processing the organism grown in said culture unit to obtain a lipid fraction; and a reactor unit for transesterifying a first lipid fraction with an alcohol to obtain alkyl esters; a separator unit to separate the alkyl esters from by-products. In one embodiment the plant further comprises a protein recovery unit for recovering protein from said organism. In another embodiment the culture unit has a growth chamber for growing the organism in a heterotrophic environment. In another embodiment the plant further comprises a filter unit for recovering and filtering the alkyl esters to obtain a bio fuel product. In another embodiment the carbonaceous material is a stillage, bagasse, stover, peel, seed cake, seed, sugar beet, a wood chip, or any combination thereof.

In another aspect, provided herein is a method for producing a biofuel product from a by-product of biofuel production, comprising the steps of: obtaining a by-product of biofuel production, said by-product of biodiesel production comprising an agricultural product; growing a evolutionary modified organism with said byproduct of biofuel production, and wherein the agricultural product is the primary carbon source; isolating and recovering the evolutionary modified organism from said growing step; fractionating the evolutionary modified organism from said growing step to obtain a first lipid fraction; transesterifying the first lipid fraction with an alcohol to obtain alkyl esters as the biofuel product; and recovering the bio fuel product. In one embodiment the organism is a bacterium, yeast, alga, or fungus. In another embodiment the organism is Chlorella protothecoides. In another embodiment the agricultural product is a glycerol, stillage, bagasse, stover, peel, seed cake, seed, sugar beet, a wood chip, or any combination thereof. In another embodiment the biofuel is a biodiesel.

In one aspect, provided herein is an organism evolutionarily modified to produce one or more end products. Also provided herein are methods and compositions for producing one or more end products. In one embodiment, the end product is a fatty acid, biofuel, biodiesel, or other valuable chemical. An EMO can be adapted to grow or use a biomass. The EMO can be adapted to grow or use any compound or material as a carbon source or nitrogen source or any other nutrient such as phosphate or sulfur. In one embodiment, the EMO can be adapted to grow or use any an agricultural product to produce one or more end products, such as a biofuel, fatty acids, biodiesel, or other valuable chemical.

In another aspect, provided herein is a method of adapting an organism for growing on stillage, comprising: placing a naturally occurring organism in a flexible tubing wherein said tubing is subdivided by an operation of a gate into one or more discreet chambers; culturing said organism; exposing said organism to stillage; and continuously culturing said organism in said chamber until said organism's growth rate or product yield on stillage is increased in comparison with the growth rate observed before exposure to said stillage. In one embodiment, said organism is a bacterium, yeast, alga, or fungus. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage.

In another aspect, provided herein is an evolved organism growing on stillage comprising: placing a naturally occurring organism in a flexible tubing wherein said tubing is subdivided by an operation of a gate into one or more discreet chambers; culturing said organism; exposing said organism to stillage; and continuously culturing said organism in said chamber until said organism's growth rate or product yield on stillage is increased in comparison with the growth rate observed before exposure to said stillage. In one embodiment, said organism is a bacterium, yeast, alga, or fungus. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage.

In another aspect, provided herein is a culture product comprising: contacting an artificially evolved microorganism with a stillage; incubating said stillage with said microorganism; and producing a culture product separable from said stillage. In one embodiment, said organism is a bacterium, yeast, alga, or fungus. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage. In another embodiment, said product is said microorganism.

In another aspect, provided herein is a method of producing an end product (such as a fatty acid or a biofuel) comprising: contacting an artificially evolved microorganism with a stillage; incubating said stillage with said microorganism; separating a cultured product from said stillage; extracting oil from said culture product; and processing said oil to biodiesel. In one embodiment, said organism is a bacterium, yeast, alga, or fungus. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage. In another embodiment, said processing is transesterfication.

In another aspect, provided herein is a method of producing biofuel comprising: contacting an artificially evolved microorganism with a stillage; incubating said stillage with said microorganism; separating a culture product from said stillage; extracting oil from said culture product; and processing said oil to biofuel. In one embodiment, said organism is a bacterium, yeast, alga, or fungus. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage. In another embodiment, said processing is cracking.

In another aspect, provided herein is a method of producing algal proteins comprising: contacting an artificially evolved microorganism with a stillage; culturing said microorganism with said stillage; and separating said microorganism from said stillage. In one embodiment, said organism is an alga. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage.

In one aspect, provided herein is a method of producing multi-vitamin supplement comprising: contacting an artificially evolved microorganism with a stillage; culturing said microorganism with said stillage; separating said microorganism from said stillage; drying said microorganism; and packaging said dried microorganism as multi-vitamins supplement. In one embodiment, said organism is an alga. In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage.

In another aspect, provided herein is a method of adapting an organism for growing on stillage, comprising: culturing said organism; exposing said organism to stillage; and continuously culturing said organism until said organism's growth rate or product yield on stillage is increased in comparison with the growth rate observed before exposure to said stillage. In one embodiment, said organism is a bacterium, yeast, alga, or fungus.

In another embodiment, said organism is Chlorella protothecoides. In another embodiment, said stillage is wet distillers grains with solubles (WDGS), dry distillers grains with solubles (DDGS), condensed distillers grains with solubles (CDS), or distillers grains (DG). In another embodiment, said stillage is hydrolyzed stillage. In another embodiment, said hydrolysis is enzymatic hydrolysis with alpha-amylase. In another embodiment, said stillage is corn stillage.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a flowchart for processing a biomass to produce fatty acids, which are subsequnetly converted into biodiesel.

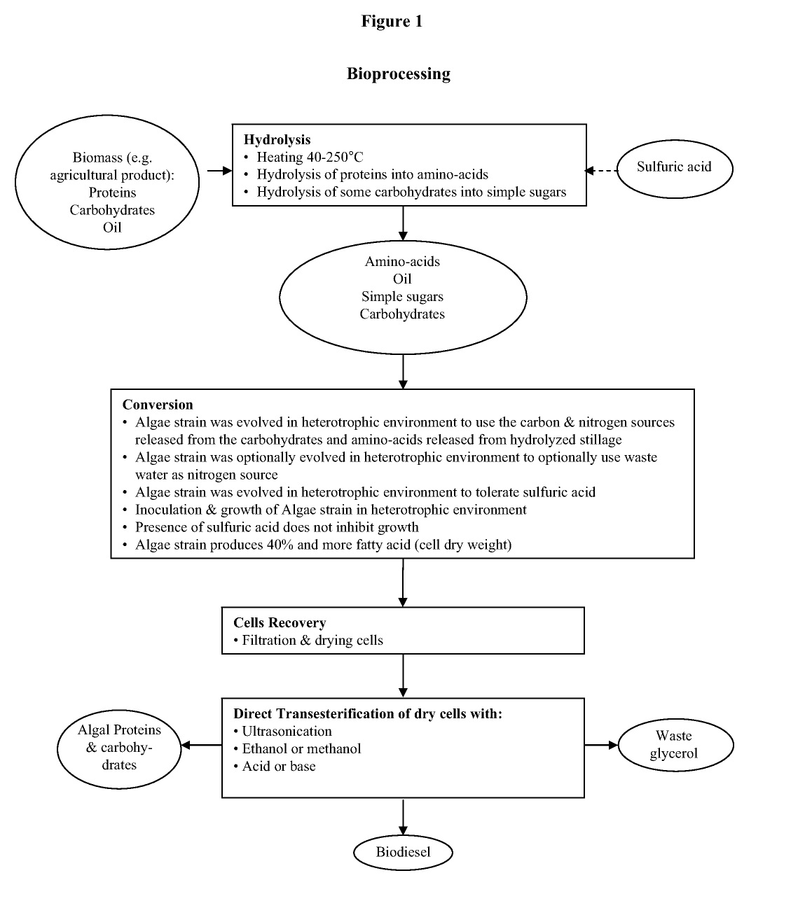

FIG. 2 illustrates a flowchart for processing corn stillage to produce fatty acids, which are subsequnetly converted into biodiesel

FIG. 3 illustrates a flowchart for processing corn stover to produce fatty acids, which are subsequnetly converted into biodiesel

FIG. 4 illustrates a flowchart for producing stillage from corn in the course of ethanol production.

FIG. 5 illustrates a flowchart depicting processing of complete jatropha seed cake with additional cellulosic compound, for fatty acid and biofuel production.

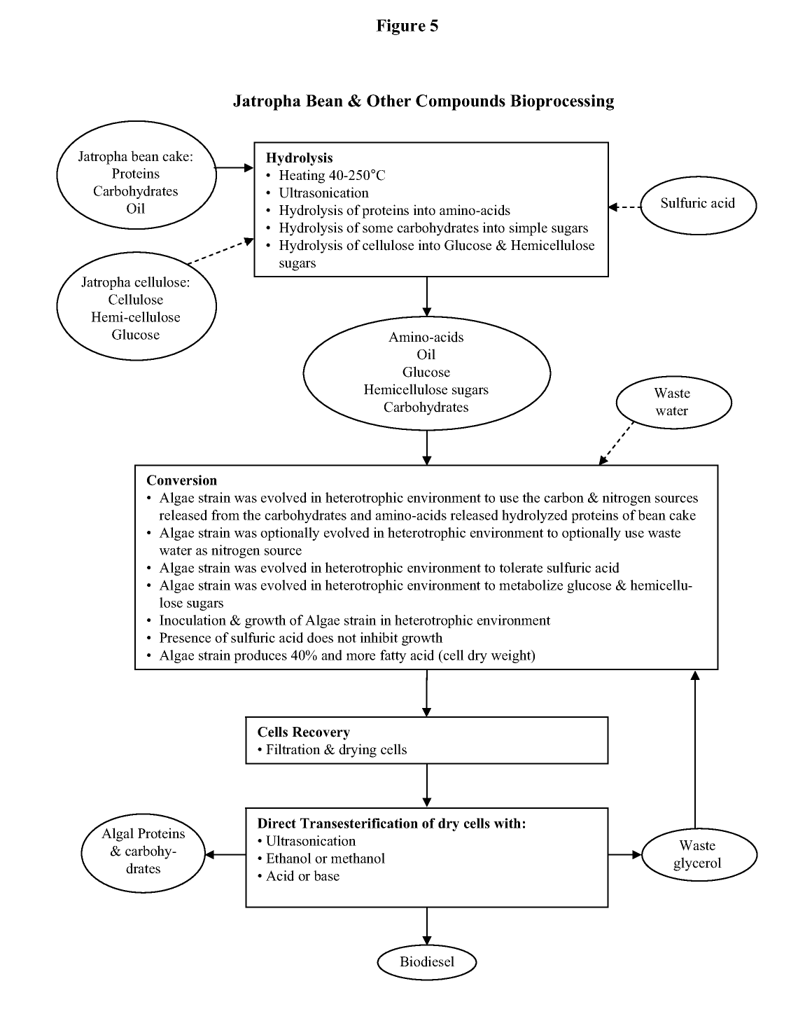

FIG. 6 illustrates a flowchart depicting processing of sole protein portion of castor seed cake, for fatty acid and biofuel production.

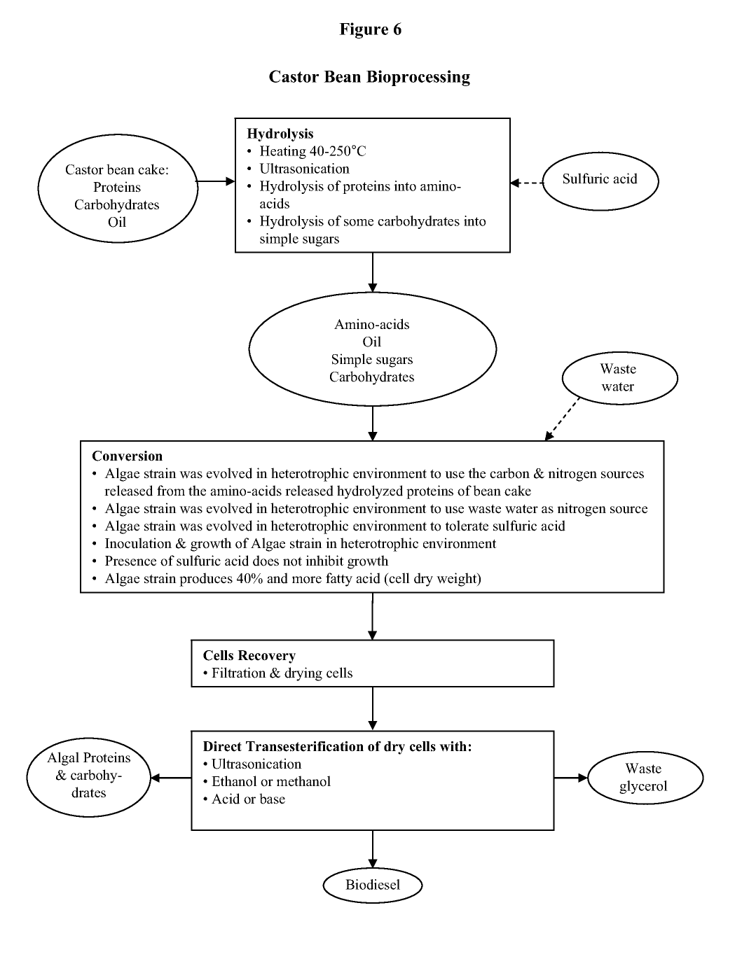

FIG. 7 illustrates a flowchart depicting processing bagasse, for fatty acid and biofuel production.

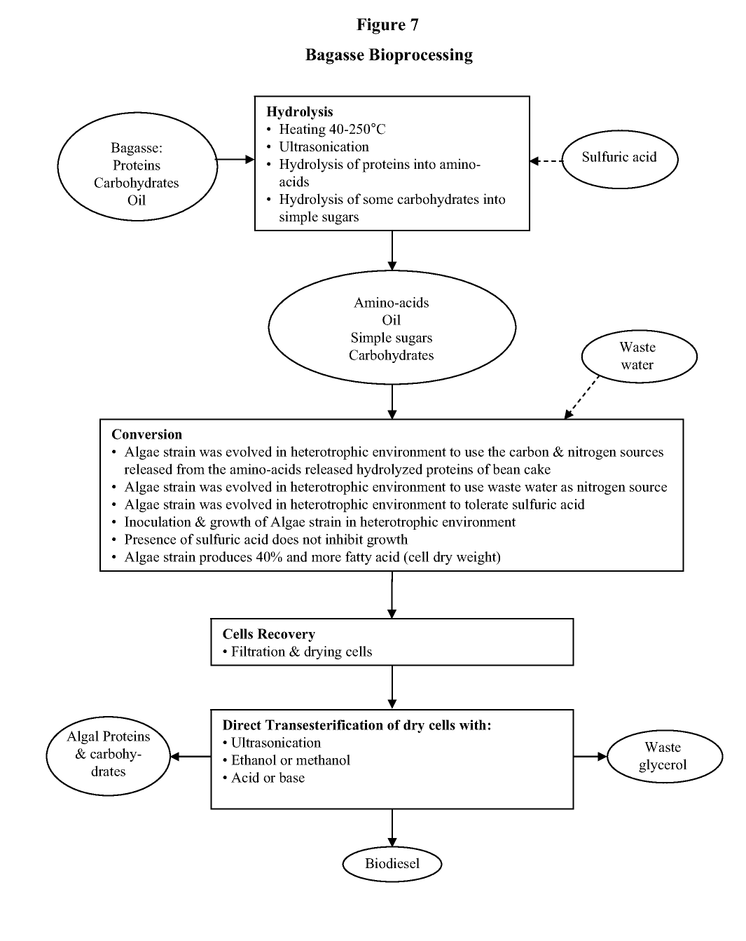

FIG. 8 illustrates a flowchart depicting an arrangement of equipment for a biofuel manufacturing site and process for producing biofuel.

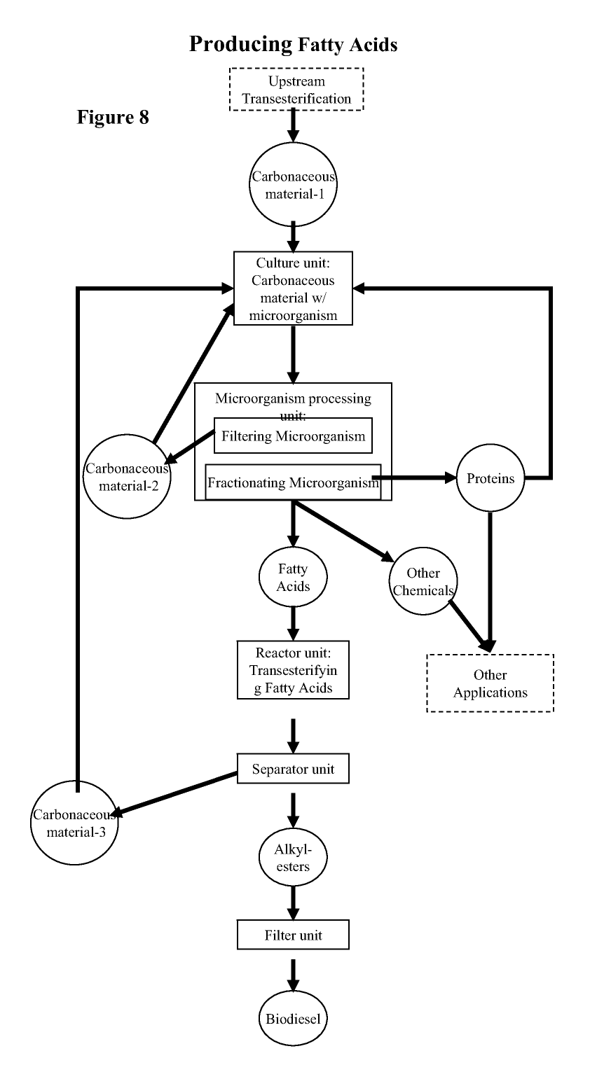

FIG. 9 illustrates a flowchart depicting an arrangement of equipment for a biofuel manufacturing site and process for producing biofuel and other hydrocarbon products.

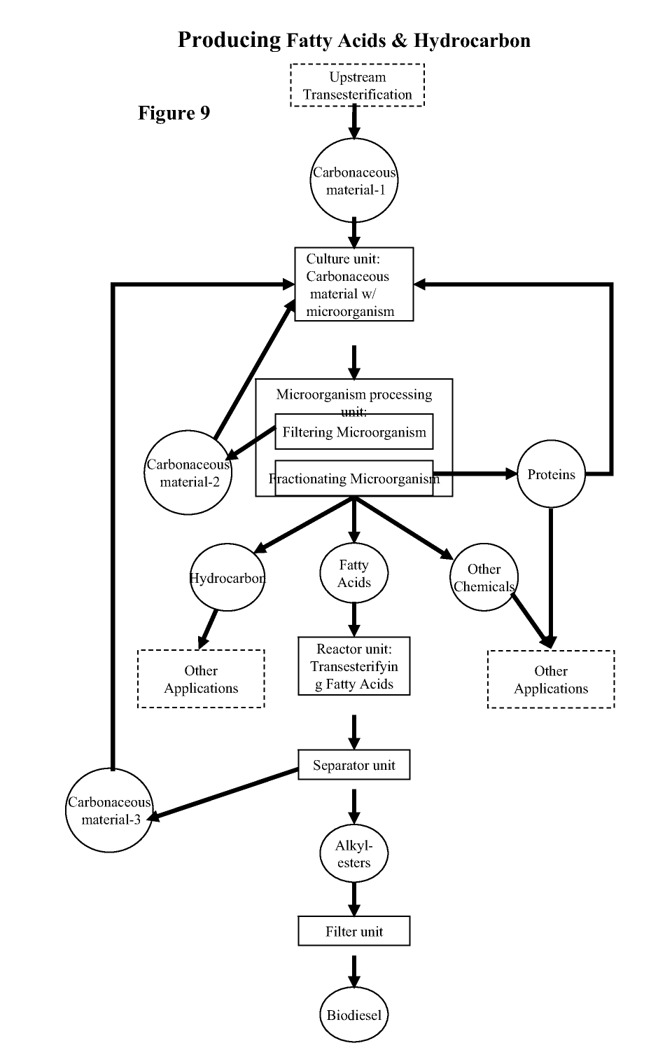

FIG. 10 illustrates the growth curves from samples obtained from the Evolugator™ showing C. protothecoides strain growing on a mix of carbohydrates, raw glycerol and Jatropha seed cake.

### DETAILED DESCRIPTION

Disclosed herein are methods and compositions of using a biomass as a source material to produce fatty acids, biofuel, biodiesel, or other useful end-products. In one embodiment, an agricultural product is utilized as a source material to produce biofuel, biodiesel, or other useful end-products. In one embodiment, an organism is evolutionarily modified to utilize carbon and/or nitogen sources available in, but not limited to, agricultural products, industrial waste, recycled waste, municipal solid waste, synthetic waste, human waste, animal waste, animal by-product, commercial organics (e.g., beverage industry waste, cheese, whey, dairy waste, food processing waste, lumber and industrial wood, waste, pulp and paper facility waste, restaurant waste, fabrics, cotton, wool and linen), construction and demolition debris, waste paper (e.g., old newspapers, old corrugated containers, mixed paper, pulp substitutes, computer printouts, white office paper, and printing pant scraps), or yard waste (e.g., leaves, twigs, grass, plant cuttings, branches, trees, and vines).

The terms “carbonaceous material” or “biomass” as used herein includes a biological materials that can be converted into a biofuel, chemical or other end product. One exemplary source of carbonaceous material is an agricultural product. One exemplary source of carbonaceous material is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, bamboo, and material derived from these. Plant matter can also be residual spent solids from alcoholic fermentation from materials such as corn and which contain lignin, starch, cellulose, hemicellulose, and proteins. Plant matter can be further described by reference to the chemical species present, such as proteins, polysaccharides (such as chitin) and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, corn stover, corn stillage, corn cobs, corn grain, bagasse, soy stems, soy leaves, soy pods, soy molasses, soy flakes, pennycress seeds or seed cake, distillers grains, peels, pits, fermentation waste, wood chips, saw dust, wood flour, wood pulp, paper pulp, paper pulp waste steams straw, lumber, sewage, seed cake, husks, rice hulls, leaves, grass clippings, food waste restaurant waste, or cooking oil. These materials can come from farms, forestry, industrial sources, households, etc. Plant matter also includes maltose, corn syrup, Distillers Dried Solubles (DDS), Distillers Dried Grains (DDG), Condensed Distillers Solubles (CDS), Distillers Wet Grains (DWG), or Distillers Dried Grains with Solubles (DDGS). Biomass iincludes animal matter such as milk, meat, fat, bone meal, animal processing waste, and animal waste (e.g., feces). “Feedstock” is frequently used to refer to biomass being used for a process, such as those described herein. Another example of carbonaceous material or biomass is glycerol (such as unpurified glycerol from a transesterification process), sewage, municipal waste, which can contain indigestible materials such as paper or other cellulosic, hemicellulosic or lignocellulosic material.

The term “fatty acid” or lipid or oil as used herein includes one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A “fatty acid” can include one or more volatile fatty acids such as acetic acid, propionic acid, butyric acid, butyrate, caproate, caprylate, valerate and heptanoate, and the like. “Fatty acids” include saturated and unsaturated fatty acids. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 30 carbons, such as 11 to 23 carbons (e.g., 8 to 30 carbons or 12 to 24 carbons counting the carboxyl group) arranged in a linear chain with or without side chains and/or substitutions. A fatty acid comprising material can be one or more of these compounds in an isolated or purified form. It can be a material that includes one or more of these compounds that is combined or blended with other similar or different materials.

The term “biofuel” as used herein includes biodiesel, biokerosene, biojet fuel, biogasoline and other hydrocarbon based fuels. A biofuel can be a liquid or a gas. A biofuel can include an alcohol, such as ethanol butanol or propanol. A biofuel also includes gases such as methane, ethane, propane, butane and H₂.

In one embodiment, the organism is evolutionarily modified to utilize carbon and/or nitogen sources available in an agricultural product. Growth of the EMO on an agricultural product can produce fatty acids, biofuel, biodiesel, or other useful end-products by a process of conversion performed by the organism described herein. Agricultural products useful for growing EMOs include any product, byproduct, or biological waste of agricultural process. In one embodiment, the agricultural product is stillage. In another embodiment, the agricultural product is a stover. In another embodiment, the agricultural product is bagasse. In another embodiment, the agricultural product is a peel. In another embodiment, the agricultural product is a seed cake. In another embodiment, the agricultural product is a seed. In another embodiment, the agricultural product is a sugar beet. In another embodiment, the agricultural product is a wood chip. In one embodiment, glycerol is a source material used to produce fatty acids, biofuel, biodiesel, or other valuable chemicals. In another embodiment an EMO uses cellulose or hemicellulose to produce an end-product such as a fuel.

Evolutionary Modification of an Organism

Provided herein is an EMO adapted to grow on a carbonaceous material. An EMO is an organism that has been artificially evolutionarily modified by culturing the organism for a sufficient period of time under controlled conditions to induce one or more changes in the organism's DNA (e.g. a point mutation, a translocation, misssense mutation, a nonsense mutation, duplication, deletion or gene rearrangement, methylation), or to induce one or more changes to the organism's chromatin (e.g., histone methylation or acetylation) that causes a desired phenotypic change. In one embodiment an EMO has been modified so that it contains multiple mutations that effect one or more genes. In another embodiment an EMO has been modified by continuous culture. In one embodiment an EMO is a single celled organism. In another embodiment an EMO is a microorganism. In another embodiment an EMO is an algae or a bacteria. In another embodiment an EMO is a multicellular organism.

In one embodiment, an EMO is adapted to grow on a specific carbonaceous material. In one embodiment, an EMO is adapted to grow on glycerol. In another embodiment, the EMO is adapted to grow on a carbonaceous material, such as industrial waste, recycled waste, municipal solid waste, synthetic waste, human waste, animal waste, animal by-product, commercial organics (e.g., beverage industry waste, cheese, whey, dairy waste, food processing waste, lumber and industrial wood, waste, pulp and paper facility waste, restaurant waste, fabrics, cotton, wool and linen), construction and demolition debris, waste paper (e.g., old newspapers, old corrugated containers, mixed paper, pulp substitutes, computer printouts, white office paper, and printing pant scraps), or yard waste (e.g., leaves, twigs, grass, plant cuttings, branches, trees, or vines), stillage, seed cake or bagasse. In one embodiment, an EMO is adapted to grow on jatropha or castor bean seed cake.

The EMO can be produced by a method, device, or composition as described herein. In one embodiment, the evolutionary modification process uses a continuous culture method described in U.S. Patent Applications 20070037276 or 20080220501, which are herein incorporated by reference in their entirety. In one embodiment an organism is evolutionarily modified with an Evolugator™ to produce an EMO specifically modified to have a desired phenotype. In another embodiment the phenotype is increased growth rate, increased production of a end product (e.g., fatty acids or alcohol), increased culture density, or decreased production of an end-product (e.g., acetate, lactate, H₂ or CO₂.

The evolutionary process described herein can be applied to a wide range of organisms. In one embodiment, an organism is evolved to grow on one or more biomasses. In one embodiment, the biomass is an agricultural product. In one embodiment, an organism evolves to acquire one or more industrially useful characteristics. In yet another embodiment, an organism is evolved to grow on one or more agricultural products and to acquire one or more industrially useful characteristics. In one embodiment, the organism is a microorganism, such as further described below.

An industrially useful characteristic can include any characteristic that enhances the production of a valuable end product, such as fatty acids, biofuel, biodiesel, or another chemical. In one embodiment, an industrially useful characteristic is enhanced growth rate on an agricultural product. In another embodiment, an industrially useful characteristic is an ability to increase the variety of end products produced. In another embodiment, an industrially useful characteristic is an ability to increase the rate of producing one or more end products. In another embodiment, an industrially useful characteristic is an ability to increase the production amount of one or more end products. In one embodiment, the end product is a chemical end product, such as further described herein. In another embodiment, the end product is a biofuel. In another embodiment, the end product is fatty acids. In yet another embodiment, the end product is a biodiesel. In yet another embodiment, the end product is a valuable chemical.

In one embodiment, the growth rate of an evolved organism is enhanced by more than about 1.5, 2, 3, 4, 5, 6, 7, 8, 9, 10, 11, 12, 13, 14, 15, 16, 17, 18, 19, 20, 25, 30, 35, 50, 45, 50, 75, or 100 times, as compared to an organism (e.g. wild-type organism, or organism from which the evolved organism was derived from) that had not been evolved. In another embodiment, the evolved organism produces a greater variety of end products as compared to an organism (e.g. wild-type organism, or organism from which the evolved organism was derived from) that had not been evolved. In one embodiment, an evolved organism produces more than about 2, 3, 4, 5, 6, 7, 8, 9, 10, 11, 12, 13, 14, 15, 16, 17, 18, 19, 20, 25, 30, 35, 50, 45, 50, 75, or 100 times the number of end products as compared to an organism (e.g. wild-type organism, or organism from which the evolved organism was derived from) that had not been evolved.

In another embodiment, the production of one or more end products by the evolved organism is increased as compared to an organism (e.g. wild-type organism, or organism from which the evolved organism was derived from) that had not been evolved. In one embodiment, an evolved organism produces an end product at a rate that is more than about 1.5, 2, 3, 4, 5, 6, 7, 8, 9, 10, 11, 12, 13, 14, 15, 16, 17, 18, 19, 20, 25, 30, 35, 50, 45, 50, 75, or 100 times the rate of the organism that had not been evolved. In another embodiment, the evolved organism produces a greater quantity of an end product as compared to an organism (e.g. wild-type organism, or organism from which the evolved organism was derived from) that had not been evolved. In one embodiment, the evolved organism produces more than about 1.5, 2, 3, 4, 5, 6, 7, 8, 9, 10, 11, 12, 13, 14, 15, 16, 17, 18, 19, 20, 25, 30, 35, 50, 45, 50, 75, or 100 times the amount of a chemical end product as compared to an organism that had not been evolved. In another embodiment, the evolved organism produces greater than about 5, 6, 7, 8, 9, 10, 11, 12, 13, 14, 15, 16, 17, 18, 19, 20, 21, 22, 23, 24, 25, 26, 27, 28, 39, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100% more of an end product as compared to an organism that had not been evolved.

An organism can be evolved to gain a characteristic including, but not limited to, enhanced growth on one or more specific nitrogen sources, carbohydrate sources, phosphorous sources, sulfur sources, or combinations thereof. In one embodiment, the various nitrogen sources, carbohydrate sources, or both are from one or more agricultural products. In another embodiment, an organism is evolved to gain a characteristic including, but not limited to, enhancing growth on various glycerol sources.

In one embodiment, a device for continuous culture is used to culture an organism disclosed herein, such as an evolving organism, EMO, or genetically modified organism. In one embodiment, the organism is a microorganism. In one embodiment, the microorganism is a bacterium, yeast, an alga, or a fungus. The culturing can be continuous ad infinitum, without causing wall growth problem. This technology allows circumventing culture problems known to be associated with wall growth and thus enable efficient and faster evolution of a microorganism toward enhancement or acquisition of industrially desirable traits through evolutionary process.

In another embodiment, continuous culturing of an organism is carried out without any fluid transfer, including sterilization or rinsing functions. In one embodiment, a continuous culture is achieved inside a flexible sterile tube filled with growth medium. In one embodiment, the medium and the chamber surface are static with respect to each other, and both are regularly and simultaneously replaced by peristaltic movement of the tubing through “gates”, or points at which the tube is sterilely subdivided by clamps that prevent the cultured cells from moving between regions of the tube. UV gates can also (optionally) be added upstream and downstream of the culture vessel for additional security.

In another embodiment, a continuous culture can select continually, rather than periodically, against adherence of dilution-resistant variants to the chemostat surfaces, as replacement of the affected surfaces occurs in tandem with the process of dilution.

In another embodiment, the flexible sterile tube employed in a continuous culture is subdivided in a transient way such that there are regions that contain saturated (fully grown) culture, regions that contain fresh medium, and a region between these two, wherein one or more chambers referred to as growth or culture chambers are present to form a growth chamber region in which grown culture is mixed with fresh medium to achieve dilution. The gates are periodically released from one point on the tube and replaced at another point, such that grown culture along with its associated growth chamber surface and attached static cells, is removed by isolation from the growth chamber and replaced by both fresh medium and fresh chamber surface.

In another embodiment, a continuous culture can proceed by repetitive movements of the gated regions of tubing. In one embodiment, this involves simultaneous movements of the gates, the tubing, the medium, and any culture within the tubing. The tubing moves in the same direction; unused tubing containing fresh medium moves into the growth chamber and mix with the culture remaining there, providing the substrate for further growth of the cells contained therein. In one embodiment, this medium and its associated tubing is maintained in a sterile condition by separation from the growth chamber by the upstream gates before introduction into the growth chamber region. Used tubing containing grown culture is simultaneously moved ‘downstream’ and separated from the growth chamber by the downstream gates. As used herein, “upstream” refers to a portion of tubing containing fresh medium and “downstream” refers to a portion of tubing containing used medium.

In another embodiment, the boundaries between upstream chamber and the growth chamber or between the growth chamber and downstream chamber are defined by gates located along the tube. Gates can be operated as clamps, either opening or closing off a section of tubing. Gates configuration, i.e., where they are located or the number of gates, or the distance between gates can be adjusted according to species-specific demand of a culture. In a given configuration, gates can be designed through one chain of multiple teeth simultaneously moved or in another configuration separated in distinct synchronized. Gates can comprise a system made of two teeth pinching the tubing in a stacking manner.

In another embodiment, when one or more growth chambers are present, the growth chambers can be used for the same or different purpose. For example, living cells could be grown in a first growth chamber and a second growth chamber with the same or different conditions. In one embodiment, a first growth chamber can be used to grow cells and a second growth chamber can then be used to treat the living cells under different conditions. For example, the cells can be treated to induce the expression of a desired product. Components or additives of the culture medium itself can be added prior to or after the culture begins. For example, all components or additives could be included in the media before beginning the culture, or components can be injected into one or more of the growth chambers after the culture have been initiated.

In another embodiment, aeration (gas exchange), is achieved directly and without mechanical assistance by the use of gas permeable tubing. In one embodiment flexible gas permeable tubing is made from silicone. Aeration could be achieved through exchange with the ambient atmosphere or through exchange with an artificially defined atmosphere (liquid or gas) that contacts the growth chamber or the entire chemostat. In embodiments where anaerobiosis is desired, the flexible tubing can be gas impermeable. For example, flexible gas impermeable tubing can be made of coated or treated silicone.

In another embodiment, anaerobic evolution conditions can be achieved by confining regions of the tubing in a specific and controlled atmospheric area to control gas exchange dynamics. This can be achieved either by making the thermostatically controlled box gastight and then injecting neutral gas into it or by placing the complete device in an atmosphere controlled room.

In another embodiment, the growing chamber can be depressurized or over pressurized according to conditions desired. Different ways of adjusting pressure can be used, for instance applying vacuum or pressurized air to the fresh medium and tubing through its upstream extremity and across the growth chamber; another way of depressurizing or over pressurizing tubing can be done by alternate pinching and locking tubing upstream of or inside the growth chamber.

In one embodiment, a continuous culture described herein can use tilting movements of the device, and/or shaking of the growth chamber by an external device to decrease aggregation of cells within the growth chamber. Alternatively, one or several stirring bars can be included in the tubing filled with fresh medium before sterilization and magnetically agitated during culture operations.

In one embodiment, continuous culture described herein can use any form of liquid or semi-solid material as a growth medium.

In another embodiment, continuous culture described herein can contain multiple growth chambers, such that the downstream gates of one growth chamber become the upstream gates of another. This could, for example, allow one cell to grow alone in the first chamber, and then act as the source of nutrition for a second cell in the second chamber.

In another embodiment, continuous culture described herein can use an emitter to subject the cells, permanently or temporarily, to one or more of radio waves, light waves, UV-radiation, x-rays, sound waves, an electro magnetic field, a radioactive field, radioactive media, or combinations thereof. The growth chamber region of the device can be subjected to, permanently or temporarily, a different gravitational force. For example, the cells can be grown in a microgravity environment.

In one embodiment, a device for culturing an organism described herein comprises a) a flexible, sterile tube containing culture medium, b) a system of clamps, each capable of open and closed positions, the clamps being positioned so as to be able to divide the tube into separate regions containing spent culture (downstream region), growing culture (growth chamber), and fresh growth medium (upstream region), c) a component for moving the clamps and the tubing such that a portion of the growth chamber and the associated culture can be clamped off and separated from the growth chamber, and such that a portion of fresh tubing containing unused medium can be joined with a portion of the culture and associated medium already present in the growth chamber, wherein each of the clamps does not move with respect to the tube when said clamp is in the closed position. In one embodiment, the tubing is flexible to allow clamping and segregation into separated chambers. In another embodiment, the tubing is gas permeable, for example comprised primarily of silicon, to allow gas exchange between the cultured organism and the outside environment, according to the conditions desired. In yet another embodiment, the tubing is gas impermeable, to prevent gas exchange between the tubing and the outside environment, if anaerobiosis is desired. In one embodiment, tubing is transparent or translucent, to allow the measurement of turbidity. In yet another embodiment, the growth chamber tubing and associated media and culture can be depressurized or over pressurized relative to ambient atmosphere as desired. In one embodiment, the tubing allows the measure of pH of medium by inclusion of a pH indicator in the tubing composition or lining. In one embodiment, the growth chamber tubing and associated media and culture can be heated or cooled as appropriate. In one embodiment, the growth chamber tubing and associated media and culture can be kept motionless or agitated by any already known method. In one embodiment, the tubing can include one or several stirring bars for agitation purpose. In another embodiment, regions of the tubing can be confined in a specific and controlled atmospheric area to control gas exchange dynamics. In another embodiment, the growth chamber tubing and associated media and culture can be tilted either downward to remove aggregated cells, or upward to remove air.

In yet another embodiment, a device for culturing an organism described herein comprises a continuous length of flexible, sterile tubing; a system of clamps positioned at points along a section of the tubing, each of the clamps being positioned and arranged so as to be able to controllably pinch the tubing by putting said clamp into a closed position in which the tubing is divided into separate regions on respective sides of said clamp, the separate regions on respective sides of the clamp being merged back into a single region when the clamp is returned to an open position; wherein the clamps and tubing are arranged so that the tubing is clamped at first through fourth points along the tubing, defining first through third regions downstream of the first through third points, respectively; and wherein a volume of the second region delimited by said points two and three is greater than a volume of the first and third regions, wherein the system of clamps is constructed so that, in a repeating pattern, the tubing is clamped upstream of the first point, the tubing is clamped at a point between the second and third points, and the second point is returned to the open position, thereby subdividing the second region into an upstream portion and a downstream portion, merging the first region and the upstream portion, and thereby defining new first through fourth points and first through third regions. In one embodiment, the tubing is gas permeable. In one embodiment, the tubing is gas impermeable. In one embodiment, the tubing is translucent. In one embodiment, the tubing is transparent. In one embodiment, the contents of the tubing in the second region can be controllably depressurized or over pressurized relative to ambient atmosphere. In one embodiment, the device further comprises a pH indicator in the tubing, a heating and cooling device that can control a temperature of contents of the tubing, an agitator, or a combination thereof. In one embodiment, the agitator comprises at least one stirring bar. In one embodiment, the regions of the tubing can be confined in a specific and controlled atmospheric area to control gas exchange dynamics. In another embodiment, the device can further comprise a device to control tilting of the second portion of the tubing.

In yet another embodiment, a device for culturing an organism described herein comprises flexible tubing containing culture medium; and a system of clamps, each capable of open and closed positions, the clamps being positioned so as to be able to divide the tubing into: i) an upstream region containing unused culture medium; i) a downstream region containing spent culture medium; and iii) a growth chamber region for growing said cells disposed between the upstream and downstream regions; wherein the system of clamps is constructed and arranged to open and close so as to clamp off and define the growth chamber region of the tubing between the upstream and downstream regions of the tubing, and to cyclically redefine the growth chamber region of the tubing so that a first portion of the previously defined growth chamber region becomes a portion of the downstream region of the tubing, and a portion of the previously defined upstream region of the tubing becomes a portion of the growth chamber region of the tubing. In one embodiment, the system of clamps is structured and arranged so that each of the clamps does not move with respect to the tubing when said clamp is in the closed position. In one embodiment, the tubing is gas permeable. In one embodiment, the tubing is gas impermeable. In one embodiment, tubing is one of transparent and translucent to permit a turbidity meter to determine the density of the culture. In yet another embodiment, the device further comprises a pressure regulator constructed to change a pressure of the growth chamber portion of the tubing relative to ambient pressure. In one embodiment, the tubing comprises a pH indicator. In yet another embodiment, the device further comprises a temperature regulator constructed to control the temperature of the growth chamber region of the tubing, an agitator constructed to allow agitation of the growth chamber portion of the tubing, or a combination thereof. In one embodiment, the agitator comprises at least one stirring bar. In yet another embodiment, the growth chamber region comprises one or more growth chambers containing culture medium.

In one embodiment a microorganism is evolutionary modified to produce higher amounts of an end-product (e.g., fatty acids or ethanol) than the same strain of non-evolutionary modified microorganism by culturing it with one or more specific carbonaceous materials.

Genetic Engineering

In one embodiment, an organism disclosed herein is genetically engineered. In contrast to evolutionarily modifying or adapting an organism, genetically engineering an organism refers to directly manipulating one or more genes of an organism, such as through molecular cloning and recombinant DNA technology. An organism can be evolutionarily modified and subsequently be genetically modified through genetic engineering. In one embodiment, an EMO can be selected and genetically engineered to have one or more characteristics. In one embodiment an EMO that is adapted to grow on a particular carbonaceous material can be genetically engineered to increase its growth rate or to decrease production of a scondary metabolite. In another embodiment, an EMO that is adapted to produce a variety of end products is genetically engineered to increase its production of one or more of the end products.

In another embodiment, an organism can be genetically engineered and then subsequently evolutionarily modified, such as for better growth rate, a higher end product yield, or both. In one embodiment, an organism can be genetically engineered to metabolize a new compound as a nutrition source and evolved to increase its growth rate on this specific new nutrient source.

In yet another embodiment, an organism can be genetically engineered to have one or more characteristics of an EMO. For example, an EMO can be selected and its genome, or a portion thereof analyzed, such as by sequencing or other techniques, to identify one or more genetic modifications of the organism's genome. The identified genetic modifications can then be expressed in another organism through genetic engineering, such as by recombinant DNA techniques. For example, an EMO can have a mutation in a gene. The mutation is identified and cloned into another organism or a wild-type or non-EMO. The mutation can be inserted into an expression cassette designed for the chosen organism and introduced into the host where it is recombinantly produced. The choice of specific regulatory sequences such as promoter, signal sequence, 5′ and 3′ untranslated sequences, and enhancer, is within the level of skill of the one ordinarily skilled in the art. The resultant molecule, containing the individual elements linked in proper orientation and reading frame, may be inserted into a vector capable of being transformed into the host cell. Suitable expression vectors and methods for recombinant production of proteins are well known for host organisms, such as for bacteria (see, e.g. Studier and Moffatt. 1986. J. Mol. Biol. 189: 113; Brosius. 1989. DNA 8: 759, for E. coli), algae (see e.g. Mayfield, S. P., S. E. Franklin, and R. A. Lerner. 2003. Expression and assembly of a fully active antibody in algae. Proc Natl Acad Sci USA 100:438-42, Zaslayskaia, L. A., J. C. Lippmeier, C. Shih, D. Ehrhardt, A. R. Grossman, and K E. Apt., 2001. Trophic conversion of an obligate photoautotrophic organism through metabolic engineering. Science 292:2073-5) and yeast (see, e.g., Schneider and Guarente. 1991. Meth. Enzymol. 194: 373).

Organism for Evolutionary Modification

In one embodiment, an organism that is evolutionarily modified is a unicellular organism. In another embodiment, an organism that is evolutionarily modified is a multicellular organism. In another embodiment, the organism is a prokaryote. In yet another embodiment, the organism is a eukaryote. In one embodiment, the organism is a microorganism. In one embodiment, the microorganism is adapted to grow on carbon and/or nitrogen sources available on agricultural products. A suitable microorganism can be a eukaryote or prokaryote, being either a bacterium, a fungus, a yeast, an alga, plant cell or an animal cell. In one embodiment, the microorganism is a bacterium. In another embodiment, the microorganism is a yeast. In another embodiment, the microorganism is a fungus. In another embodiment, the microorganism is an alga.

In another embodiment a microorganism utilized herein is evolutionarily modified to serve as an improved bioagent for producing biofuel, fatty acids, biodiesel, hydrocarbon product or other fermentation or bioconversion end-product.

In another embodiment a microorganism can be a strain different than its wild type, which can be identified by the mutations acquired during the course of culture, and these mutations, can allow the new cells to be distinguished from their ancestor's genotype characteristics. Thus, one can select new strains of microorganisms by segregating individual strains with improved rates of reproduction through the process of natural selection.

In another embodiment a microorganism can be evolutionarily modified in a number of ways so that their growth rate, viability, and utility as a biofuel, fatty acids, biodiesel, or other hydrocarbon source of product can be improved. For example, a microorganism can be evolutionarily modified to enhance its ability to grow on a particular substrate, to use a particular carbonaceous material, to use a particluar phosphorus source, to use a particluar sulfur source, to use a particluar energy source, to use a particluar nitrogen source, to tolerate a component of a substrate/material/source that could be inhibiting growth or end product yield, or to tolerate specific environmental conditions (such as high or low pH, pressure, temperature or oxygenation).

In another embodiment a microorganism suitable for methods described herein can be naturally occurring or genetically modified. For example, algae can be cultivated for use as fatty acids, biofuel, biodiesel, or hydrocarbon based product.

In one embodiment, an alga is a heterotrophic alga, which can be grown in a closed reactor.

While a variety of algal species can be used, algae that naturally contain a high amount of lipids, such as 15-90%, 30-80%, 40-60%, and 25-60% by dry weight of the algae is useful for production of fatty acids, biofuel or biodiesel.

Algae useful for methods and compositions described herein include, but are not limited to, Chlorophyta (Chlorella and Prototheca), Prasinophyta (Dunaliella), Bacillariophyta (Navicula and Nitzschia), Ochrophyta (Ochromonas), Dinophyta (Gyrodinium) and Euglenozoa (Euglena). In one embodiment, Chlorella protothecoides is evolutionarily modified to grow on stillage. In another embodiment, Dunaliella Salina is modified to grow on agricultural products.

Cyanobacteria are prokaryotes (single-celled organisms) often referred to as “blue-green algae.” Cyanobacteria can also be used for evolutionary modification as described herein.

Bacteria can also be evolutionarily modified to produce a biofuel comprising lipids, hydrocarbons such as phytanyl and dibiphytanyl molecules and fatty acids that are naturally expressed and produced by the microorganisms. It can also be selected to produce fermentation products such as acetate, acetone, 2,3-butanediol, butanol, butyrate, CO₂, ethanol, formate, glycolate, lactate, malate, propionate, pyruvate, succinate, and other fermentation products. In one embodiment, a bacterium is selected from Corynobacterium, Nocardia, or Mycobacterium. In another embodiment, the bacterium is Mycobacterium smegmatis.

In one embodiment a EMO is a fungus. A fungus can be evolutionarily modified to enzymatically digest lignin or to acquire the ability to an increased rate of lignin digestion compared to that of a wild type species. In one embodiment, an evolutionarily modified fungus is a species of white rot fungi. Examples of white rot fungi include, but are not limited to, Bjerkandera adusta, Ceriporiopsis subvermispora, Coriolus versicolor, Corolopsis gallica, Corolopsis polyzona, Gloeophyllum striatum, Gloeophyllum trabeum, Irpex lacteus, Kuehneromyces mutabilis, Laetiporus sulphureus, Nematoloma frowardii, Phanerochaete chrysosporium, Phanerochaete laevis, Phanerochaete sordida, Phlebia radiata, Phlebia tremellosa, Pleurotus eryngii, Pleurotus ostreatus, Pleurotus pulmonarius, Pycnoporus cinnabarinus, Trametes hirsutus, Trametes hispida, or Trametes versicolor. In another embodiment, an evolutionarily modified fungus is Trichoderma reesei. In another embodiment, an evolutionarily modified fungus is Phanerochaete chrysosporium.

In one embodiment, a fungal strain is evolutionarily modified to break down lignin obtained from bagasse. In one embodiment, the fungal stain is Lentinula edodes. In another embodiment, the fungal stain is Pleurotus eryngii. In another embodiment, the fungal stain is Ceriporiopsis subvermispora.

In one embodiment, the microorganism to be evolutionarily modified is a Clostridium, including but not limited to, C. acetobutylicum, C. aerotolerans, C. baratii, C. beijerinckii, C. bifermentans, C. botulinum, C. butyricum, C. cadaveric, C. chauvoei, C. clostridioforme, C. colicanis, C. difficile, C. fallax, C. feseri, C. formicaceticum, C. histolyticum, C. innocuum, C. kluyveri, C. ljungdahlii, C. laramie, C. lavalense, C. nigrificans, C. novyi, C. oedematiens, C. paraputrificum, C. perfringens, C. phytofermentans, C. piliforme, C. ramosum, C. scatologenes, C. septicum, C. sordellii, C. sporogenes, C. tertium, C. tetani, C. thermocellum, or C. tyrobutyricum.

In yet another embodiment, the microorganism useful for evolutionarily modifications include, but are not limited to, Chaetomiaceae such as the genera Chaetomium e.g. the species Chaetomidium fimeti; Choanephoraceae such as the genera Blakeslea, Choanephora e.g. the species Blakeslea trispora, Choanephora cucurbitarum or Choanephora infundibulifera var. cucurbitarum; Cryptococcaceae such as the genera Candida, Crytococcus, Rhodotorula, Torulopsis e.g. the species Candida albicans, Candida albomarginata, Candida antarctica, Candida bacarum, Candida bogoriensis, Candida boidinii, Candida bovina, Candida brumptii, Candida cacaoi, Candida cariosilignicola, Candida catenulata, Candida chalmersii, Candida ciferrii, Candida cylindracea, Candida edax, Candida ernobii, Candida famata, Candida freyschussii, Candida friedrichii, Candida glabrata, Candida guilliermondii, Candida haemulonii, Candida humicola, Candida inconspicua, Candida ingens, Candida intermedia, Candida kefyr, Candida krusei, Candida lactiscondensi, Candida lambica, Candida lipolytica, Candida lusitaniae, Candida macedoniensis, Candida magnoliae, Candida membranaefaciens, Candida mesenterica, Candida multigemmis, Candida mycoderma, Candida nemodendra, Candida nitratophila, Candida norvegensis, Candida norvegica, Candida parapsilosis, Candida pelliculosa, Candida peltata, Candida pini, Candida pseudotropicalis, Candida pulcherrima, Candida punicea, Candida pustula, Candida ravautii, Candida reukaufii, Candida rugosa, Candida sake, Candida silvicola, Candida solani, Candida sp., Candida spandovensis, Candida succiphila, Candida tropicalis, Candida utilis, Candida valida, Candida versatilis, Candida vini, Candida zeylanoides, Cryptococcus albidus, Cryptococcus curvatus, Cryptococcus flavus, Cryptococcus humicola, Cryptococcus hungaricus, Cryptococcus kuetzingii, Cryptococcus laurentii, Cryptococcus macerans, Cryptococcus neoformans, Cryptococcus terreus, Cryptococcus uniguttulatus, Rhodotorula acheniorum, Rhodotorula bacarum, Rhodotorula bogoriensis, Rhodotorula flava, Rhodotorula glutinis, Rhodotorula macerans, Rhodotorula minuta, Rhodotorula mucilaginosa, Rhodotorula pilimanae, Rhodotorula pustula, Rhodotorula rubra, Rhodotorula tokyoensis, Torulopsis colliculosa, Torulopsis dattila or Torulopsis neoformans; Cunninghamellaceae such as the genera Cunninghamella e.g. the species Cunninghamella blakesleeana, Cunninghamella echinulata, Cunninghamella echinulata var. elegans, Cunninghamella elegans or Cunninghamella homothallica; Demetiaceae such as the genera Alternaria, Bipolaris, Cercospora, Chalara, Cladosporium, Curvularia, Exophilia, Helicosporium, Helminthosporium, Orbimyces, Philalophora, Pithomyces, Spilocaea, Thielaviopsis, Wangiella e.g. the species Curvularia affinis, Curvularia clavata, Curvularia fallax, Curvularia inaequalis, Curvularia indica, Curvularia lunata, Curvularia pallescens, Curvularia verruculosa or Helminothosporium sp.; Moniliaceae such as the genera Arthrobotrys, Aspergillus, Epidermophyton, Geotrichum, Gliocladium, Histoplasma, Microsporum, Monilia, Oedocephalum, Oidium, Penicillium, Trichoderma, Trichophyton, Thrichoteclum, Verticillium e.g. the species Aspergillus aculeatus, Aspergillus albus, Aspergillus alliaceus, Aspergillus asperescens, Aspergillus awamori, Aspergillus candidus, Aspergillus carbonarius, Aspergillus carneus, Aspergillus chevalieri, Aspergillus chevalieri var. intermedius, Aspergillus clavatus, Aspergillus ficuum, Aspergillus flavipes, Aspergillus flavus, Aspergillus foetidus, Aspergillus fumigatus, Aspergillus giganteus, Aspergillus humicola, Aspergillus intermedius, Aspergillus japonicus, Aspergillus nidulans, Aspergillus niger, Aspergillus niveus, Aspergillus ochraceus, Aspergillus oryzae, Aspergillus ostianus, Aspergillus parasiticus, Aspergillus parasiticus var. globosus, Aspergillus penicillioides, Aspergillus phoenicis, Aspergillus rugulosus, Aspergillus sclerotiorum, Aspergillus sojae var. gymnosardae, Aspergillus sydowi, Aspergillus tamarii, Aspergillus terreus, Aspergillus terricola, Aspergillus toxicarius, Aspergillus unguis, Aspergillus ustus, Aspergillus versicolor, Aspergillus vitricolae, Aspergillus wentii, Penicillium adametzi, Penicllium albicans, Penicillium arabicum, Penicillium arenicola, Penicillium argillaceum, Penicillium arvense, Penicillium asperosporum, Penicillium aurantiogriseum, Penicillium avellaneum, Penicillium baarnense, Penicillium bacillisporum, Penicillium brasilianum, Penicillium brevicompactum, Penicillium camemberti, Penicillium canadense, Penicillium canescens, Penicillium caperatum, Penicillium capsulatum, Penicillium caseicolum, Penicillium chrysogenum, Penicillium citreonigrum, Penicillium citrinum, Penicillium claviforme, Penicillium commune, Penicillium corylophilum, Penicillium corymbiferum, Penicillium crustosum, Penicillium cyclopium, Penicillium daleae, Penicillium decumbens, Penicillium dierckxii, Penicillium digitatum, Penicillium digitatum var. latum, Penicillium divaricatum, Penicillium diversum, Penicillium duclauxii, Penicillium echinosporum, Penicillium expansum, Penicillium fellutanum, Penicillium frequentans, Penicillium funiculosum, Penicillium glabrum, Penicillium gladioli, Penicillium griseofulvum, Penicillium hirsutum, Penicillium hispanicum, Penicillium islandicum, Penicillium italicum, Penicillium italicum var. avellaneum, Penicillium janczewskii, Penicillium janthinellum, Penicillium japonicum, Penicillium lavendulum, Penicillium iilacinum, Penicillium iividum, Penicillium martensii, Penicillium megasporum, Penicillium miczynskii, Penicillium nalgiovense, Penicillium nigricans, Penicillium notatum, Penicillium ochrochloron, Penicillium odoratum, Penicillium oxalicum, Penicillium paraherquei, Penicillium patulum, Penicillium pinophilum, Penicillium piscarium, Penicillium pseudostromaticum, Penicillium puberulum, Penicillium purpurogenum, Penicillium raciborskii, Penicillium roqueforti, Penicillium rotundum, Penicillium rubrum, Penicillium sacculum, Penicillium simplicissimum, Penicillium sp., Penicillium spinulosum, Penicillium steckii, Penicillium stoloniferum, Penicillium striatisporum, Penicillium striatum, Penicillium tardum, Penicillium thomii, Penicillium turbatum, Penicillium variabile, Penicillium vermiculatum, Penicillium vermoesenii, Penicillium verrucosum, Penicillium verrucosum var. corymbiferum, Penicillium verrucosum var. cyclopium, Penicillium verruculosum, Penicillium vinaceum, Penicillium violaceum, Penicillium viridicatum, Penicillium vulpinum, Trichoderma hamatum, Trichoderma harzianum, Trichoderma koningii, Trichoderma longibrachiatum, Trichoderma polysporum, Trichoderma reesei, Trichoderma vixens or Trichoderma viride; Mortierellaceae such as the genera Mortierella e.g. the species Mortierella isabellina, Mortierella polycephala, Mortierella ramanniana, Mortierella vinacea or Mortierella zonata; Mucoraceae such as the genera Actinomucor, Mucor, Phycomyces, Rhizopus, Zygorhynchus e.g. the species Mucor amphibiorum, Mucor circinelloides f. circinelloides, Mucor circinelloides var. griseocyanus, Mucor flavus, Mucor fuscus, Mucor griseocyanus, Mucor heterosporus, Mucor hiemalis, Mucor hiemalis f. hiemalis, Mucor inaequisporus, Mucor indicus, Mucor javanicus, Mucor mucedo, Mucor mucilagineus, Mucor piriformis, Mucor plasmaticus, Mucor plumbeus, Mucor racemosus, Mucor racemosus f. racemosus, Mucor racemosus f. sphaerosporus, Mucor rouxianus, Mucor rouxii, Mucor sinensis, Mucor sp., Mucor spinosus, Mucor tuberculisporus, Mucor variisporus, Mucor variosporus, Mucor wosnessenskii, Phycomyces blakesleeanus, Rhizopus achlamydosporus, Rhizopus arrhizus, Rhizopus chinensis, Rhizopus delemar, Rhizopus formosaensis, Rhizopus japonicus, Rhizopus javanicus, Rhizopus microsporus, Rhizopus microsporus var. chinensis, Rhizopus microsporus var. oligosporus, Rhizopus microsporus var. rhizopodiformis, Rhizopus nigricans, Rhizopus niveus, Rhizopus oligosporus, Rhizopus oryzae, Rhizopus pygmaeus, Rhizopus rhizopodiformis, Rhizopus semarangensis, Rhizopus sontii, Rhizopus stolonifer, Rhizopus thermosus, Rhizopus tonkinensis, Rhizopus tritici or Rhizopus usamii; Pythiaceae such as the genera Phytium, Phytophthora e.g. the species Pythium debaryanum, Pythium intermedium, Pythium irregulare, Pythium megalacanthum, Pythium paroecandrum, Pythium sylvaticum, Pythium ultimum, Phytophthora cactorum, Phytophthora cinnamomi, Phytophthora citricola, Phytophthora citrophthora, Phytophthora cryptogea, Phytophthora drechsleri, Phytophthora erythroseptica, Phytophthora lateralis, Phytophthora megasperma, Phytophthora nicotianae, Phytophthora nicotianae var. parasitica, Phytophthora palmivora, Phytophthora parasitica or Phytophthora syringae; Sacharomycetaceae such as the genera Hansenula, Pichia, Saccharomyces, Saccharomycodes, Yarrowia e.g. the species Hansenula anomala, Hansenula californica, Hansenula canadensis, Hansenula capsulata, Hansenula ciferńi, Hansenula glucozyma, Hansenula henricii, Hansenula holstii, Hansenula minuta, Hansenula nonfermentans, Hansenula philodendri, Hansenula polymorphs, Hansenula saturnus, Hansenula subpelliculosa, Hansenula wickerhamii, Hansenula wingei, Pichia alcoholophila, Pichia angusta, Pichia anomala, Pichia bispora, Pichia burtonii, Pichia canadensis, Pichia capsulata, Pichia carsonii, Pichia cellobiosa, Pichia ciferrii, Pichia farinosa, Pichia fermentans, Pichia finlandica, Pichia glucozyma, Pichia guilliermondii, Pichia haplophila, Pichia henricii, Pichia holstii, Pichia jadinii, Pichia lindnerii, Pichia membranaefaciens, Pichia methanolica, Pichia minuta var. minuta, Pichia minuta var. nonfermentans, Pichia norvegensis, Pichia ohmeri, Pichia pastoris, Pichia philodendri, Pichia pini, Pichia polymorpha, Pichia quercuum, Pichia rhodanensis, Pichia sargentensis, Pichia stipitis, Pichia strasburgensis, Pichia subpelliculosa, Pichia toletana, Pichia trehalophila, Pichia vini, Pichia xylosa, Saccharomyces aceti, Saccharomyces bailii, Saccharomyces bayanus, Saccharomyces bisporus, Saccharomyces capensis, Saccharomyces carlsbergensis, Saccharomyces cerevisiae, Saccharomyces cerevisiae var. ellipsoideus, Saccharomyces chevalieri, Saccharomyces delbrueckii, Saccharomyces diastaticus, Saccharomyces drosophilaeum, Saccharomyces elegans, Saccharomyces ellipsoideus, Saccharomyces fermentati, Saccharomyces florentinus, Saccharomyces fragilis, Saccharomyces heterogenous, Saccharomyces hienipiensis, Saccharomyces inusitatus, Saccharomyces italicus, Saccharomyces kiuyveri, Saccharomyces krusei, Saccharomyces lactis, Saccharomyces marxianus, Saccharomyces microellipsoides, Saccharomyces montanus, Saccharomyces norbensis, Saccharomyces oleaceus, Saccharomyces paradoxus, Saccharomyces pastorianus, Saccharomyces pretoriensis, Saccharomyces rosei, Saccharomyces rouxii, Saccharomyces uvarum, Saccharomycodes ludwigii or Yarrowia lipolytica; Saprolegniaceae such as the genera Saprolegnia e.g. the species Saprolegnia ferax; Schizosacharomycetaceae such as the genera Schizosaccharomyces e.g. the species Schizosaccharomyces japonicus var. japonicus, Schizosaccharomyces japonicus var. versatilis, Schizosaccharomyces malidevorans, Schizosaccharomyces octosporus, Schizosaccharomyces pombe var. malidevorans or Schizosaccharomyces pombe var. pombe; Sodariaceae such as the genera Neurospora, Sordaria e.g. the species Neurospora africana, Neurospora crassa, Neurospora intermedia, Neurospora sitophila, Neurospora tetrasperma, Sordaria fimicola or Sordaria macrospora; Tuberculariaceae such as the genera Epicoccum, Fusarium, Myrothecium, Sphacelia, Starkeyomyces, Tubercularia e.g. the species Fusarium acuminatum, Fusarium anthophilum, Fusarium aquaeductuum, Fusarium aquaeductuum var. medium, Fusarium avenaceum, Fusarium buharicum, Fusarium camptoceras, Fusarium cerealis, Fusarium chlamydosporum, Fusarium ciliatum, Fusarium coccophilum, Fusarium coeruleum, Fusarium concolor, Fusarium crookwellense, Fusarium culmorum, Fusarium dimerum, Fusarium diversisporum, Fusarium equiseti, Fusarium equiseti var. bullatum, Fusarium eumartii, Fusarium flocciferum, Fusarium fujikuroi, Fusarium graminearum, Fusarium graminum, Fusarium heterosporum, Fusarium incarnatum, Fusarium inflexum, Fusarium javanicum, Fusarium lateritium, Fusarium lateritium var. majus, Fusarium longipes, Fusarium melanochlorum, Fusarium merismoides, Fusarium merismoides var. chlamydosporale, Fusarium moniliforme, Fusarium moniliforme var. anthophilum, Fusarium moniliforme var. subglutinans, Fusarium nivale, Fusarium nivale var. majus, Fusarium oxysporum, Fusarium oxysporum f. sp. aechmeae, Fusarium oxysporum f. sp. cepae, Fusarium oxysporum f. sp. conglutinans, Fusarium oxysporum f. sp. cucumerinum, Fusarium oxysporum f. sp. cyclaminis, Fusarium oxysporum f. sp. dianthi, Fusarium oxysporum f. sp. lycopersici, Fusarium oxysporum f. sp. melonis, Fusarium oxysporum f. sp. passflorae, Fusarium oxysporum f. sp. pisi, Fusarium oxysporum f. sp. tracheiphilum, Fusarium oxysporum f. sp. tuberosi, Fusarium oxysporum f. sp. tulipae, Fusarium oxysporum f. sp. vasinfectum, Fusarium pallidoroseum, Fusarium poae, Fusarium proliferatum, Fusarium proliferatum var. minus, Fusarium redolens, Fusarium redolens f. sp. dianthi, Fusarium reticulatum, Fusarium roseum, Fusarium sacchari var. elongatum, Fusarium sambucinum, Fusarium sambucinum var. coeruleum, Fusarium semitectum, Fusarium semitectum var. majus, Fusarium solani, Fusarium solani f. sp. pisi, Fusarium sporotrichioides, Fusarium sporotrichioides var. minus, Fusarium sublunatum, Fusarium succisae, Fusarium sulphureum, Fusarium tabacinum, Fusarium tricinctum, Fusarium udum, Fusarium ventricosum, Fusarium verticillioides, Fusarium xylarioides or Fusarium zonatum; Sporobolomycetaceae such as the genera Bullera, Sporobolomyces, ltersonilia e.g. the species Sporobolomyces holsaticus, Sporobolomyces odorus, Sporobolomyces puniceus, Sporobolomyces salmonicolor, Sporobolomyces singularis or Sporobolomyces tsugae; Adelotheciaceae such as the genera e.g. the species Physcomitrella patens; Dinophyceae such as the genera Crypthecodinium, Phaeodactylum e.g. the species Crypthecodinium cohnii or Phaeodactylum tricornutum; Ditrichaceae such as the genera Ceratodon, Pleuridium, Astomiopsis, Ditrichum, Philibertiella, Ceratodon, Distichium, Skottsbergia e.g. the species Ceratodon antarcticus, Ceratodon purpureus, Ceratodon purpureus ssp. convolutes or Ceratodon purpureus ssp. stenocarpus; Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri; Actinomycetaceae such as the genera Actinomyces, Actinobaculum, Arcanobacterium, Mobiluncus e.g. the species Actinomyces bernardiae, Actinomyces bovis, Actinomyces bowdenii, Actinomyces canis, Actinomyces cardiffensis, Actinomyces catuli, Actinomyces coleocanis, Actinomyces denticolens, Actinomyces europaeus, Actinomyces funkei, Actinomyces georgiae, Actinomyces gerencsehae, Actinomyces hordeovulnehs, Actinomyces howellii, Actinomyces humiferus, Actinomyces hyovaginalis, Actinomyces israelii, Actinomyces marimamma\ddot{v}um, Actinomyces meyeri, Actinomyces naeslundii, Actinomyces nasicola, Actinomyces neuii subsp. anitratus, Actinomyces neuii subsp. neuii, Actinomyces odontolyticus, Actinomyces oricola, Actinomyces pyogenes, Actinomyces radicidentis, Actinomyces radingae, Actinomyces slackii, Actinomyces suimastitidis, Actinomyces suis, Actinomyces turicensis, Actinomyces urogenitalis, Actinomyces vaccimaxillae, Actinomyces viscosus, Actinobaculum schaalii, Actinobaculum suis, Actinobaculum urinate, Arcanobacterium bernardiae, Arcanobacterium haemolyticum, Arcanobactehum hippocoleae, Arcanobacterium phocae, Arcanobacterium pluranimalium, Arcanobacterium pyogenes, Mobiluncus curtisii subsp. curtisii, Mobiluncus curtisii subsp. holmesii or Mobiluncus mulieris; Bacillaceae such as the genera Amphibacillus, Anoxybacillus, Bacillus, Exiguobacterium, Gracilibacillus, Holobacillus, Saccharococcus, Salibacillus, Virgibacillus e.g. the species Amphibacillus fermentum, Amphibacillus tropicus, Amphibacillus xylanus, Anoxybacillus flavithermus, Anoxybacillus gonensis, Anoxybacillus pushchinoensis, Bacillus acidocaldarius, Bacillus acidoterrestris, Bacillus aeolius, Bacillus agaradhaerens, Bacillus agri, Bacillus alcalophilus, Bacillus alginolyticus, Bacillus alvei, Bacillus amyloliquefaciens, Bacillus amylolyticus, Bacillus aneurinilyticus, Bacillus aquimaris, Bacillus arseniciselenatis, Bacillus atrophaeus, Bacillus azotofixans, Bacillus azotoformans, Bacillus badius, Bacillus barbaricus, Bacillus benzoevorans, Bacillus borstelensis, Bacillus brevis, Bacillus carboniphilus, Bacillus centrosporus, Bacillus cereus, Bacillus chitinolyticus, Bacillus chondroitinus, Bacillus choshinensis, Bacillus circulans, Bacillus clarkii, Bacillus clausii, Bacillus coagulans, Bacillus cohnii, Bacillus curdlanolyticus, Bacillus cycloheptanicus, Bacillus decolorationis, Bacillus dipsosauri, Bacillus edaphicus, Bacillus ehimensis, Bacillus endophytics, Bacillus fastidiosus, Bacillus firmus, Bacillus flexus, Bacillus formosus, Bacillus fumarioli, Bacillus funiculus, Bacillus fusiformis, Bacillus sphaericus subsp. fusiformis, Bacillus galactophilus, Bacillus globisporus, Bacillus globisporus subsp. marinus, Bacillus glucanolyticus, Bacillus gordonae, Bacillus halmapalus, Bacillus haloalkaliphilus, Bacillus halodenitrificans, Bacillus halodurans, Bacillus halophilus, Bacillus horikoshii, Bacillus horti, Bacillus infernos, Bacillus insolitus, Bacillus jeotgali, Bacillus kaustophilus, Bacillus kobensis, Bacillus krulwichiae, Bacillus laevolacticus, Bacillus larvae, Bacillus laterosporus, Bacillus lautus, Bacillus lentimorbus, Bacillus lentus, Bacillus licheniformis, Bacillus luciferensis, Bacillus macerans, Bacillus macquariensis, Bacillus marinus, Bacillus marisflavi, Bacillus marismortui, Bacillus megaterium, Bacillus methanolicus, Bacillus migulanus, Bacillus mojavensis, Bacillus mucilaginosus, Bacillus mycoides, Bacillus naganoensis, Bacillus nealsonii, Bacillus neidei, Bacillus niacini, Bacillus okuhidensis, Bacillus oleronius, Bacillus pabuli, Bacillus pallidus, Bacillus pantothenticus, Bacillus parabrevis, Bacillus pasteurii, Bacillus peoriae, Bacillus polymyxa, Bacillus popilliae, Bacillus pseudalcaliphilus, Bacillus pseudofirmus, Bacillus pseudomycoides, Bacillus psychrodurans, Bacillus psychrophilus, Bacillus psychrosaccharolyticus, Bacillus psychrotolerans, Bacillus pulvifaciens, Bacillus pumilus, Bacillus pycnus, Bacillus reuszeri, Bacillus salexigens, Bacillus schlegelii, Bacillus selenitireducens, Bacillus silvestris, Bacillus simplex, Bacillus siralis, Bacillus smithii, Bacillus sonorensis, Bacillus sphaericus, Bacillus sporothermodurans, Bacillus stearothermophilus, Bacillus subterraneus, Bacillus subtilis subsp. spizizenii, Bacillus subtilis subsp. subtilis, Bacillus thermantarcticus, Bacillus thermoaerophilus, Bacillus thermoamylovorans, Bacillus thermoantarcticus, Bacillus thermocatenulatus, Bacillus thermocloacae, Bacillus thermodenitrificans, Bacillus thermoglucosidasius, Bacillus thermoleovorans, Bacillus thermoruber, Bacillus thermosphaericus, Bacillus thiaminolyticus, Bacillus thuringiensis, Bacillus tusciae, Bacillus validus, Bacillus vallismortis, Bacillus vedderi, Bacillus vulcani, Bacillus weihenstephanensis, Exiguobacterium acetylicum, Exiguobacterium antarcticum, Exiguobacterium aurantiacum, Exiguobacterium undae, Gracilibacillus dipsosauri, Gracilibacillus halotolerans, Halobacillus halophilus, Halobacillus karajensis, Halobacillus litoralis, Halobacillus salinus, Halobacillus trueperi, Saccharococcus caldoxylosilyticus, Saccharococcus thermophilus, Salibacillus marismortui, Salibacillus salexigens, Virgibacillus carmonensis, Virgibacillus marismortui, Virgibacillus necropolis, Virgibacillus pantothenticus, Virgibacillus picturae, Virgibacillus proomii or Virgibacillus salexigens, Brevibacteriaceae such as the genera Brevibacterium e.g. the species Brevibacterium acetylicum, Brevibacterium albidum, Brevibacterium ammoniagenes, Brevibacterium avium, Brevibacterium casei, Brevibacterium citreum, Brevibacterium divahcatum, Brevibacterium epidermidis, Brevibacterium fermentans, Brevibacterium frigoritolerans, Brevibacterium halotolerans, Brevibacterium imperiale, Brevibacterium incertum, Brevibacterium iodinum, Brevibacterium linens, Brevibacterium liquefaciens, Brevibacterium lutescens, Brevibacterium luteum, Brevibacterium lyticum, Brevibacterium mcbrellneri, Brevibacterium otitidis, Brevibacterium oxydans, Brevibacterium paucivorans, Brevibacterium protophormiae, Brevibacterium pusillum, Brevibacterium saperdae, Brevibacterium stationis, Brevibacterium testaceum or Brevibacterium vitaeruminis; Corynebacteriaceae such as the genera Corynebacterium e.g. the species Corynebacterium accolens, Corynebacterium afermentans subsp. afermentans, Corynebacterium afermentans subsp. lipophilum, Corynebacterium ammoniagenes, Corynebacterium amycolatum, Corynebacterium appendicis, Corynebacterium aquilae, Corynebacterium argentoratense, Corynebacterium atypicum, Corynebacterium aurimucosum, Corynebacterium auris, Corynebacterium auriscanis, Corynebacterium betae, Corynebacterium beticola, Corynebacterium bovis, Corynebacterium callunae, Corynebacterium camporealensis, Corynebacterium capitovis, Corynebacterium casei, Corynebacterium confusum, Corynebacterium coyleae, Corynebacterium cystitidis, Corynebacterium durum, Corynebacterium efficiens, Corynebacterium equi, Corynebacterium falsenii, Corynebacterium fascians, Corynebacterium felinum, Corynebacterium flaccumfaciens, Corynebacterium flavescens, Corynebacterium freneyi, Corynebacterium glaucum, Corynebacterium glucuronolyticum, Corynebacterium glutamicum, Corynebacterium hoagii, Corynebacterium ilicis, Corynebacterium imitans, Corynebacterium insidiosum, Corynebacterium iranicum, Corynebacterium jeikeium, Corynebacterium kroppenstedtii, Corynebacterium kutscheri, Corynebacterium lilium, Corynebacterium lipophiloflavum, Corynebacterium macginleyi, Corynebacterium mastitidis, Corynebacterium matruchotii, Corynebacterium michiganense, Corynebacterium michiganense subsp. tessellarius, Corynebacterium minutissimum, Corynebacterium mooreparkense, Corynebacterium mucifaciens, Corynebacterium mycetoides, Corynebacterium nebraskense, Corynebacterium oortii, Corynebacterium paurometabolum, Corynebacterium phocae, Corynebacterium pilosum, Corynebacterium poinsettiae, Corynebacterium propinquum, Corynebacterium pseudodiphtheriticum, Corynebacterium pseudotuberculosis, Corynebacterium pyogenes, Corynebacterium rathayi, Corynebacterium renale, Corynebacterium riegelii, Corynebacterium seminale, Corynebacterium sepedonicum, Corynebacterium simulans, Corynebacterium singulare, Corynebacterium sphenisci, Corynebacterium spheniscorum, Corynebacterium striatum, Corynebacterium suicordis, Corynebacterium sundsvallense, Corynebacterium terpenotabidum, Corynebacterium testudinoris, Corynebacterium thomssenii, Corynebacterium tritici, Corynebacterium ulcerans, Corynebacterium urealyticum, Corynebacterium variabile, Corynebacterium vitaeruminis or Corynebacterium xerosis; Enterobacteriacae such as the genera Alterococcus, Arsenophonus, Brenneria, Buchnera, Budvicia, Buttiauxella, Calymmatobacterium, Cedecea, Citrobacter, Edwardsiella, Enterobacter, Erwinia, Escherichia, Ewingella, Hafnia, Klebsiella, Kluyvera, Leclercia, Leminorella, Moellerella, Morganella, Obesumbacterium, Pantoea, Pectobacterium, Photorhabdus, Plesiomonas, Pragia, Proteus, Providencia, Rahnella, Saccharobacter, Salmonella, Shigella, Serratia, Sodalis, Tatumella, Trabulsiella, Wigglesworthia, Xenorhabdus, Yersinia and Yokenella e.g. the species Arsenophonus nasoniae, Brenneria alni, Brenneria nigrifiuens, Brenneria quercina, Brenneria rubrifaciens, Brenneria salicis, Budvicia aquatica, Buttiauxella agrestis, Buttiauxella brennerae, Buttiauxella ferragutiae, Buttiauxella gaviniae, Buttiauxella izardii, Buttiauxella noackiae, Buttiauxella warmboldiae, Cedecea davisae, Cedecea lapagei, Cedecea neteri, Citrobacter amalonaticus, Citrobacter diversus, Citrobacter freundii, Citrobacter genomospecies, Citrobacter gillenii, Citrobacter intermedium, Citrobacter koseri, Citrobacter murliniae, Citrobacter sp., Edwardsiella hoshinae, Edwardsiella ictaluri, Edwardsiella tarda, Erwinia alni, Erwinia amylovora, Erwinia ananatis, Erwinia aphidicola, Erwinia billingiae, Erwinia cacticida, Erwinia cancerogena, Erwinia carnegieana, Erwinia carotovora subsp. atroseptica, Erwinia carotovora subsp. betavasculorum, Erwinia carotovora subsp. odohfera, Erwinia carotovora subsp. wasabiae, Erwinia chrysanthemi, Erwinia cypripedii, Erwinia dissolvens, Erwinia herbicola, Erwinia mallotivora, Erwinia milletiae, Erwinia nigrifluens, Erwinia nimipressuralis, Erwinia persicina, Erwinia psidii, Erwinia pyrifoliae, Erwinia quercina, Erwinia rhapontici, Erwinia rubrifaciens, Erwinia salicis, Erwinia stewartii, Erwinia tracheiphila, Erwinia uredovora, Escherichia adecarboxylata, Escherichia anindolica, Escherichia aurescens, Escherichia blattae, Escherichia coli, Escherichia coli var. communior, Escherichia coli-mutabile, Escherichia fergusonii, Escherichia hermannii, Escherichia sp., Escherichia vulneris, Ewingella americana, Hafnia alvei, Klebsiella aerogenes, Klebsiella edwardsii subsp. atlantae, Klebsiella ornithinolytica, Klebsiella oxytoca, Klebsiella planticola, Klebsiella pneumoniae, Klebsiella pneumoniae subsp. pneumoniae, Klebsiella sp., Klebsiella terrigena, Klebsiella trevisanii, Kluyvera ascorbata, Kluyvera citrophila, Kluyvera cochleae, Kluyvera cryocrescens, Kluyvera georgiana, Kluyvera noncitrophila, Kluyvera sp., Leclercia adecarboxylata, Leminorella grimontii, Leminorella richardii, Moellerella wisconsensis, Morganella morganii, Morganella morganii subsp. morganii, Morganella morganii subsp. sibonii, Obesumbaterium proteus, Pantoea agglomerans, Pantoea ananatis, Pantoea citrea, Pantoea dispersa, Pantoea punctata, Pantoea stewartii subsp. stewartii, Pantoea terrea, Pectobacterium atrosepticum, Pectobacterium carotovorum subsp. atrosepticum, Pectobacterium carotovorum subsp. carotovorum, Pectobacterium chrysanthemi, Pectobacterium cypripedii, Photorhabdus asymbiotica, Photorhabdus luminescens, Photorhabdus luminescens subsp. akhurstii, Photorhabdus luminescens subsp. laumondii, Photorhabdus luminescens subsp. luminescens, Photorhabdus sp., Photorhabdus temperata, Plesiomonas shigelloides, Pragia fontium, Proteus hauseri, Proteus ichthyosmius, Proteus inconstans, Proteus mirabilis, Proteus morganii, Proteus myxofaciens, Proteus penneri, Proteus rettgeri, Proteus shigelloides, Proteus vulgaris, Providencia alcalifaciens, Providencia friedericiana, Providencia heimbachae, Providencia rettgeri, Providencia rustigianii, Providencia stuartii, Rahnella aquatilis, Salmonella abony, Salmonella arizonae, Salmonella bongori, Salmonella choleraesuis subsp. arizonae, Salmonella choleraesuis subsp. bongori, Salmonella choleraesuis subsp. cholereasuis, Salmonella choleraesuis subsp. diarizonae, Salmonella choleraesuis subsp. houtenae, Salmonella choleraesuis subsp. indica, Salmonella choleraesuis subsp. salamae, Salmonella daressalaam, Salmonella enterica subsp. houtenae, Salmonella enterica subsp. salamae, Salmonella entehtidis, Salmonella gallinarum, Salmonella heidelberg, Salmonella panama, Salmonella senftenberg, Salmonella typhimurium, Serratia entomophila, Serratia ficaria, Serratia fonticola, Serratia grimesii, Serratia liquefaciens, Serratia marcescens, Serratia marcescens subsp. marcescens, Serratia marinorubra, Serratia odorifera, Serratia plymouthensis, Serratia plymuthica, Serratia proteamaculans, Serratia proteamaculans subsp. quinovora, Serratia quinivorans, Serratia rubidaea, Shigella boydii, Shigella flexneri, Shigella paradysenteriae, Shigella sonnei, Tatumella ptyseos, Xenorhabdus beddingii, Xenorhabdus bovienii, Xenorhabdus luminescens, Xenorhabdus nematophila, Xenorhabdus nematophila subsp. beddingii, Xenorhabdus nematophila subsp. bovienii, Xenorhabdus nematophila subsp. poinarii or Xenorhabdus poinarii; Gordoniaceae such as the genera Gordonia, Skermania e.g. the species Gordonia aichiensis, Gordonia alkanivorans, Gordonia amarae, Gordonia amicalis, Gordonia bronchialis, Gordonia desulfuricans, Gordonia hirsuta, Gordonia hydrophobica, Gordonia namibiensis, Gordonia nitida, Gordonia paraffinivorans, Gordonia polyisoprenivorans, Gordonia rhizosphera, Gordonia rubripertincta, Gordonia sihwensis, Gordonia sinesedis, Gordonia sputi, Gordonia terrae or Gordonia westfalica; Micrococcaceae such as the genera Micrococcus, Arthrobacter, Kocuria, Nesterenkonia, Renibacterium, Rothia, Stomatococcus e.g. the species Micrococcus agilis, Micrococcus antarcticus, Micrococcus halobius, Micrococcus kristinae, Micrococcus luteus, Micrococcus lylae, Micrococcus nishinomiyaensis, Micrococcus roseus, Micrococcus sedentarius, Micrococcus varians, Arthrobacter agilis, Arthrobacter albus, Arthrobacter atrocyaneus, Arthrobacter aurescens, Arthrobacter chlorophenolicus, Arthrobacter citreus, Arthrobacter creatinolyticus, Arthrobacter crystallopoietes, Arthrobacter cumminsii, Arthrobacter duodecadis, Arthrobacter flavescens, Arthrobacter flavus, Arthrobacter gandavensis, Arthrobacter globiformis, Arthrobacter histidinolovorans, Arthrobacter ilicis, Arthrobacter koreensis, Arthrobacter luteolus, Arthrobacter methylotrophus, Arthrobacter mysorens, Arthrobacter nasiphocae, Arthrobacter nicotianae, Arthrobacter nicotinovorans, Arthrobacter oxydans, Arthrobacter pascens, Arthrobacter picolinophilus, Arthrobacter polychromogenes, Arthrobacter protophormiae, Arthrobacter psychrolactophilus, Arthrobacter radiotolerans, Arthrobacter ramosus, Arthrobacter rhombi, Arthrobacter roseus, Arthrobacter siderocapsulatus, Arthrobacter simplex, Arthrobacter sulfonivorans, Arthrobacter sulfureus, Arthrobacter terregens, Arthrobacter tumescens, Arthrobacter uratoxydans, Arthrobacter ureafaciens, Arthrobacter variabilis, Arthrobacter viscosus, Arthrobacter woluwensis, Kocuria erythromyxa, Kocuria kristinae, Kocuria palustris, Kocuria polaris, Kocuria rhizophila, Kocuria rosea, Kocuria varians, Nesterenkonia halobia, Nesterenkonia lacusekhoensis, Renibacterium salmoninarum, Rothia amarae, Rothia dentocariosa, Rothia mucilaginosa, Rothia nasimurium or Stomatococcus mucilaginosus; Mycobacteriaceae such as the genera Mycobacterium e.g. the species Mycobacterium africanum, Mycobacterium agri, Mycobacterium aichiense, Mycobacterium alvei, Mycobacterium asiaticum, Mycobacterium aurum, Mycobacterium austroafricanum, Mycobacterium bohemicum, Mycobacterium botniense, Mycobacterium brumae, Mycobacterium chelonae subsp. abscessus, Mycobacterium chitae, Mycobacterium chlorophenolicum, Mycobacterium chubuense, Mycobacterium confluentis, Mycobacterium cookii, Mycobacterium diernhoferi, Mycobacterium doricum, Mycobacterium duvalii, Mycobacterium fallax, Mycobacterium farcinogenes, Mycobacterium flavescens, Mycobacterium frederiksbergense, Mycobacterium gadium, Mycobacterium gilvum, Mycobacterium gordonae, Mycobacterium hassiacum, Mycobacterium hiberniae, Mycobacterium hodleri, Mycobacterium holsaticum, Mycobacterium komossense, Mycobacterium lacus, Mycobacterium madagascariense, Mycobacterium mageritense, Mycobacterium montefiorense, Mycobacterium moriokaense, Mycobacterium murale, Mycobacterium neoaurum, Mycobacterium nonchromogenicum, Mycobacterium obuense, Mycobacterium palustre, Mycobacterium parafortuitum, Mycobacterium peregrinum, Mycobacterium phlei, Mycobacterium pinnipedii, Mycobacterium poriferae, Mycobacterium pulveris, Mycobacterium rhodesiae, Mycobacterium shottsii, Mycobacterium sphagni, Mycobacterium terrae, Mycobacterium thermoresistibile, Mycobacterium tokaiense, Mycobacterium triviale, Mycobacterium tusciae or Mycobacterium vanbaalenii; Nocardiaceae such as the genera Nocardia, Rhodococcus e.g. the species Nocardia abscessus, Nocardia africana, Nocardia amarae, Nocardia asteroides, Nocardia autotrophica, Nocardia beijingensis, Nocardia brasiliensis, Nocardia brevicatena, Nocardia caishijiensis, Nocardia calcarea, Nocardia carnea, Nocardia cellulans, Nocardia cerradoensis, Nocardia coeliaca, Nocardia corynebacterioides, Nocardia crassostreae, Nocardia cummidelens, Nocardia cyriacigeorgica, Nocardia farcinica, Nocardia flavorosea, Nocardia fluminea, Nocardia globerula, Nocardia hydrocarbonoxydans, Nocardia ignorata, Nocardia mediterranei, Nocardia nova, Nocardia orientalis, Nocardia otitidis-caviarum, Nocardia otitidiscaviarum, Nocardia paucivorans, Nocardia petroleophila, Nocardia pinensis, Nocardia pseudobrasiliensis, Nocardia pseudovaccinii, Nocardia puris, Nocardia restricta, Nocardia rugosa, Nocardia salmonicida, Nocardia saturnea, Nocardia seriolae, Nocardia soli, Nocardia sulphurea, Nocardia transvalensis, Nocardia uniformis, Nocardia vaccinii, Nocardia veterana or Nocardia vinacea; Pseudomonaceae such as the genera Azomonas, Azotobacter, Cellvibrio, Chryseomonas, Flaviomojras, Lampropedia, Mesophilobacter, Morococcus, Oligella, Pseudomonas, Rhizobacter, Rugamonas, Serpens, Thermoleophilum, Xylophilus e.g. the species Azomonas agilis, Azomonas insignis, Azomonas macrocytogenes, Azotobacter agilis, Azotobacter agilis subsp. armeniae, Azotobacter armeniacus, Azotobacter beijerinckii, Azotobacter chroococcum, Azotobacter indicum, Azotobacter macrocytogenes, Azotobacter miscellum, Azotobacter nigricans subsp. nigricans, Azotobacter paspali, Azotobacter salinestris, Azotobacter sp., Azotobacter vinelandii, Flavimonas oryzihabitans, Mesophilobacter marinus, Oligella urethralis, Pseudomonas acidovorans, Pseudomonas aeruginosa, Pseudomonas agarici, Pseudomonas alcaligenes, Pseudomonas aminovorans, Pseudomonas amygdali, Pseudomonas andropogonis, Pseudomonas anguilliseptica, Pseudomonas antarctica, Pseudomonas antimicrobica, Pseudomonas antimycetica, Pseudomonas aptata, Pseudomonas an/illa, Pseudomonas asplenii, Pseudomonas atlantica, Pseudomonas atrofaciens, Pseudomonas aureofaciens, Pseudomonas avellanae, Pseudomonas azelaica, Pseudomonas azotocolligans, Pseudomonas balearica, Pseudomonas harken, Pseudomonas bathycetes, Pseudomonas beijerinckii, Pseudomonas brassicacearum, Pseudomonas brenneri, Pseudomonas butanovora, Pseudomonas carboxydoflava, Pseudomonas carboxydohydrogena, Pseudomonas carboxydovorans, Pseudomonas carrageenovora, Pseudomonas caryophylli, Pseudomonas cepacia, Pseudomonas chlohtidismutans, Pseudomonas chlororaphis, Pseudomonas cichorii, Pseudomonas citronellolis, Pseudomonas cocovenenans, Pseudomonas compransoris, Pseudomonas congelans, Pseudomonas coronafaciens, Pseudomonas corrugata, Pseudomonas dacunhae, Pseudomonas delafieldii, Pseudomonas delphinii, Pseudomonas denitrificans, Pseudomonas desmolytica, Pseudomonas diminuta, Pseudomonas doudoroffli, Pseudomonas echinoides, Pseudomonas elongata, Pseudomonas extorquens, Pseudomonas extremorientalis, Pseudomonas facilis, Pseudomonas ficuserectae, Pseudomonas flava, Pseudomonas flavescens, Pseudomonas fluorescens, Pseudomonas fragi, Pseudomonas frederiksbergensis, Pseudomonas fulgida, Pseudomonas fuscovaginae, Pseudomonas gazotropha, Pseudomonas gladioli, Pseudomonas glathei, Pseudomonas glumae, Pseudomonas graminis, Pseudomonas halophila, Pseudomonas helianthi, Pseudomonas huttiensis, Pseudomonas hydrogenothermophila, Pseudomonas hydrogenovora, Pseudomonas indica, Pseudomonas indigofera, Pseudomonas iodinum, Pseudomonas kilonensis, Pseudomonas lachrymans, Pseudomonas lapsa, Pseudomonas lemoignei, Pseudomonas lemonnieri, Pseudomonas lundensis, Pseudomonas luteola, Pseudomonas maltophilia, Pseudomonas marginalis, Pseudomonas marginata, Pseudomonas marina, Pseudomonas meliae, Pseudomonas mendocina, Pseudomonas mesophilica, Pseudomonas mixta, Pseudomonas monteilii, Pseudomonas morsprunorum, Pseudomonas multivorans, Pseudomonas natriegens, Pseudomonas nautica, Pseudomonas nitroreducens, Pseudomonas oleovorans, Pseudomonas oryzihabitans, Pseudomonas ovalis, Pseudomonas oxalaticus, Pseudomonas palleronii, Pseudomonas paucimobilis, Pseudomonas phaseolicola, Pseudomonas phenazinium, Pseudomonas pickettii, Pseudomonas pisi, Pseudomonas plantarii, Pseudomonas plecoglossicida, Pseudomonas poae, Pseudomonas primulae, Pseudomonas proteolytica, Pseudomonas pseudoalcaligenes, Pseudomonas pseudoalcaligenes subsp. konjaci, Pseudomonas pseudoalcaligenes subsp. pseudoalcaligenes, Pseudomonas pseudoflava, Pseudomonas putida, Pseudomonas putida var. naraensis, Pseudomonas putrefaciens, Pseudomonas pyrrocinia, Pseudomonas radiora, Pseudomonas reptilivora, Pseudomonas rhodesiae, Pseudomonas rhodos, Pseudomonas hboflavina, Pseudomonas rubescens, Pseudomonas rubrisubalbicans, Pseudomonas ruhlandii, Pseudomonas saccharophila, Pseudomonas savastanoi, Pseudomonas savastanoi pvar. glycinea, Pseudomonas savastanoi pvar. phaseolicola, Pseudomonas solanacearum, Pseudomonas sp., Pseudomonas spinosa, Pseudomonas stanieri, Pseudomonas stutzeri, Pseudomonas syringae, Pseudomonas syringae pvar. aptata, Pseudomonas syringae pvar. atrofaciens, Pseudomonas syringae pvar. coronafaciens, Pseudomonas syringae pvar. delphinii, Pseudomonas syringae pvar. glycinea, Pseudomonas syringae pvar. helianthi, Pseudomonas syringae pvar. lachrymans, Pseudomonas syringae pvar. lapsa, Pseudomonas syringae pvar. morsprunorum, Pseudomonas syringae pvar. phaseolicola, Pseudomonas syringae pvar. primulae, Pseudomonas syringae pvar. syringae, Pseudomonas syringae pvar. tabaci, Pseudomonas syringae pvar. tomato, Pseudomonas syringae subsp. glycinea, Pseudomonas syringae subsp. savastanoi, Pseudomonas syringae subsp. syringae, Pseudomonas syzygii, Pseudomonas tabaci, Pseudomonas taeniospiralis, Pseudomonas testosteroni, Pseudomonas thermocarboxydovorans, Pseudomonas thermotolerans, Pseudomonas thivervalensis, Pseudomonas tomato, Pseudomonas trivialis, Pseudomonas veronii, Pseudomonas vesicularis, Pseudomonas viridiflava, Pseudomonas viscogena, Pseudomonas woodsii, Rhizobacter dauci, Rhizobacter daucus or Xylophilus ampelinus; Rhizobiaceae such as the genera Agrobacterium, Carbophilus, Chelatobacter, Ensifer, Rhizobium, Sinorhizobium e.g. the species Agrobacterium atlanticum, Agrobacterium ferrugineum, Agrobacterium gelatinovorum, Agrobacterium larrymoorei, Agrobacterium meteori, Agrobacterium radiobacter, Agrobacterium rhizogenes, Agrobacterium rubi, Agrobacterium stellulatum, Agrobacterium tumefaciens, Agrobacterium vitis, Carbophilus carboxidus, Chelatobacter heintzii, Ensifer adhaerens, Ensifer arboris, Ensifer fredii, Ensifer kostiensis, Ensifer kummerowiae, Ensifer medicae, Ensifer meliloti, Ensifer saheli, Ensifer terangae, Ensifer xinjiangensis, Rhizobium ciceri Rhizobium etli, Rhizobium fredii, Rhizobium galegae, Rhizobium gallicum, Rhizobium giardinii, Rhizobium hainanense, Rhizobium huakuii, Rhizobium huautlense, Rhizobium indigoferae, Rhizobium japonicum, Rhizobium leguminosarum, Rhizobium loessense, Rhizobium loti, Rhizobium lupini, Rhizobium mediterraneum, Rhizobium meliloti, Rhizobium mongolense, Rhizobium phaseoli, Rhizobium radiobacter, Rhizobium rhizogenes, Rhizobium rubi, Rhizobium sullae, Rhizobium tianshanense, Rhizobium trifolii, Rhizobium tropici, Rhizobium undicola, Rhizobium vitis, Sinorhizobium adhaerens, Sinorhizobium arboris, Sinorhizobium fredii, Sinorhizobium kostiense, Sinorhizobium kummerowiae, Sinorhizobium medicae, Sinorhizobium meliloti, Sinorhizobium morelense, Sinorhizobium saheli or Sinorhizobium xinjiangense; Streptomycetaceae such as the genera Kitasatosprora, Streptomyces, Streptoverticillium e.g. the species Streptomyces abikoensis, Streptomyces aburaviensis, Streptomyces achromogenes subsp. achromogenes, Streptomyces achromogenes subsp. rubradiris, Streptomyces acidiscabies, Streptomyces acrimycini, Streptomyces aculeolatus, Streptomyces afghaniensis, Streptomyces alanosinicus, Streptomyces albaduncus, Streptomyces albiaxialis, Streptomyces albidochromogenes, Streptomyces albidoflavus, Streptomyces albireticuli, Streptomyces albofaciens, Streptomyces alboflavus, Streptomyces albogriseolus, Streptomyces albolongus, Streptomyces alboniger, Streptomyces albospinus, Streptomyces albosporeus subsp. albosporeus, Streptomyces albosporeus subsp. labilomyceticus, Streptomyces alboverticillatus, Streptomyces albovinaceus, Streptomyces alboviridis, Streptomyces albulus, Streptomyces albus subsp. albus, Streptomyces albus subsp. pathocidicus, Streptomyces almquistii, Streptomyces althioticus, Streptomyces amakusaensis, Streptomyces ambofaciens, Streptomyces aminophilus, Streptomyces anandii, Streptomyces anthocyanicus, Streptomyces antibioticus, Streptomyces antimycoticus, Streptomyces anulatus, Streptomyces arabicus, Streptomyces ardus, Streptomyces arenae, Streptomyces argenteolus, Streptomyces armeniacus, Streptomyces asiaticus, Streptomyces asterosporus, Streptomyces atratus, Streptomyces atroaurantiacus, Streptomyces atroolivaceus, Streptomyces atrovirens, Streptomyces aurantiacus, Streptomyces aurantiogriseus, Streptomyces aureocirculatus, Streptomyces aureofaciens, Streptomyces aureorectus, Streptomyces aureoversilis, Streptomyces aureoverticillatus, Streptomyces aureus, Streptomyces avellaneus, Streptomyces avermectinius, Streptomyces avermitilis, Streptomyces avidinii, Streptomyces azaticus, Streptomyces azureus, Streptomyces baarnensis, Streptomyces bacillaris, Streptomyces badius, Streptomyces baldaccii, Streptomyces bambergiensis, Streptomyces beijiangensis, Streptomyces bellus, Streptomyces bikiniensis, Streptomyces biverticillatus, Streptomyces blastmyceticus, Streptomyces bluensis, Streptomyces bobili, Streptomyces bottropensis, Streptomyces brasiliensis, Streptomyces bungoensis, Streptomyces cacaoi subsp. asoensis, Streptomyces cacaoi subsp. cacaoi, Streptomyces caelestis, Streptomyces caeruleus, Streptomyces californicus, Streptomyces calvus, Streptomyces canaries, Streptomyces candidus, Streptomyces canescens, Streptomyces cangkringensis, Streptomyces caniferus, Streptomyces canus, Streptomyces capillispiralis, Streptomyces capoamus, Streptomyces carpaticus, Streptomyces carpinensis, Streptomyces catenulae, Streptomyces caviscabies, Streptomyces cavourensis subsp. cavourensis, Streptomyces cavourensis subsp. washingtonensis, Streptomyces cellostaticus, Streptomyces celluloflavus, Streptomyces cellulolyticus, Streptomyces cellulosae, Streptomyces champavatii, Streptomyces chartreuses, Streptomyces chattanoogensis, Streptomyces chibaensis, Streptomyces chrestomyceticus, Streptomyces chromofuscus, Streptomyces chryseus, Streptomyces chrysomallus subsp. chrysomallus, Streptomyces chrysomallus subsp. fumigatus, Streptomyces cinereorectus, Streptomyces cinereoruber subsp. cinereoruber, Streptomyces cinereoruber subsp. fructofermentans, Streptomyces cinereospinus, Streptomyces cinereus, Streptomyces cinerochromogenes, Streptomyces cinnabarinus, Streptomyces cinnamonensis, Streptomyces cinnamoneus, Streptomyces cinnamoneus subsp. albosporus, Streptomyces cinnamoneus subsp. cinnamoneus, Streptomyces cinnamoneus subsp. lanosus, Streptomyces cinnamoneus subsp. sparsus, Streptomyces cirratus, Streptomyces ciscaucasicus, Streptomyces citreofluorescens, Streptomyces clavifer, Streptomyces clavuligerus, Streptomyces cochleatus, Streptomyces coelescens, Streptomyces coelicoflavus, Streptomyces coelicolor, Streptomyces coeruleoflavus, Streptomyces coeruleofuscus, Streptomyces coeruleoprunus, Streptomyces coeruleorubidus, Streptomyces coerulescens, Streptomyces collinus, Streptomyces colombiensis, Streptomyces corchorusii, Streptomyces costaricanus, Streptomyces cremeus, Streptomyces crystallinus, Streptomyces curacoi, Streptomyces cuspidosporus, Streptomyces cyaneofuscatus, Streptomyces cyaneus, Streptomyces cyanoalbus, Streptomyces cystargineus, Streptomyces daghestanicus, Streptomyces diastaticus subsp. ardesiacus, Streptomyces diastaticus subsp. diastaticus, Streptomyces diastatochromogenes, Streptomyces distallicus, Streptomyces djakartensis, Streptomyces durhamensis, Streptomyces echinatus, Streptomyces echinoruber, Streptomyces ederensis, Streptomyces ehimensis, Streptomyces endus, Streptomyces enissocaesilis, Streptomyces erumpens, Streptomyces erythraeus, Streptomyces erythrogriseus, Streptomyces eurocidicus, Streptomyces europaeiscabiei, Streptomyces eurythermus, Streptomyces exfoliates, Streptomyces felleus, Streptomyces fervens, Streptomyces fervens subsp. fervens, Streptomyces fervens subsp. melrosporus, Streptomyces filamentosus, Streptomyces filipinensis, Streptomyces fimbriatus, Streptomyces fimicarius, Streptomyces finlayi, Streptomyces flaveolus, Streptomyces flaveus, Streptomyces flavidofuscus, Streptomyces flavidovirens, Streptomyces flaviscleroticus, Streptomyces flavofungini, Streptomyces flavofuscus, Streptomyces flavogriseus, Streptomyces flavopersicus, Streptomyces flavotricini, Streptomyces flavovariabilis, Streptomyces flavovirens, Streptomyces flavoviridis, Streptomyces flocculus, Streptomyces floridae, Streptomyces fluorescens, Streptomyces fradiae, Streptomyces fragilis, Streptomyces fulvissimus, Streptomyces fulvorobeus, Streptomyces fumanus, Streptomyces fumigatiscleroticus, Streptomyces galbus, Streptomyces galilaeus, Streptomyces gancidicus, Streptomyces gardneri, Streptomyces gelaticus, Streptomyces geysiriensis, Streptomyces ghanaensis, Streptomyces gibsonii, Streptomyces glaucescens, Streptomyces glaucosporus, Streptomyces glaucus, Streptomyces globisporus subsp. caucasicus, Streptomyces globisporus subsp. flavofuscus, Streptomyces globisporus subsp. globisporus, Streptomyces globosus, Streptomyces glomeratus, Streptomyces glomeroaurantiacus, Streptomyces gobitricini, Streptomyces goshikiensis, Streptomyces gougerotii, Streptomyces graminearus, Streptomyces graminofaciens, Streptomyces ghseinus, Streptomyces griseoaurantiacus, Streptomyces griseobrunneus, Streptomyces griseocarneus, Streptomyces griseochromogenes, Streptomyces griseoflavus, Streptomyces griseofuscus, Streptomyces griseoincarnatus, Streptomyces griseoloalbus, Streptomyces griseolosporeus, Streptomyces griseolus, Streptomyces griseoluteus, Streptomyces griseomycini, Streptomyces griseoplanus, Streptomyces griseorubens, Streptomyces griseoruber, Streptomyces griseorubiginosus, Streptomyces griseosporeus, Streptomyces griseostramineus, Streptomyces griseoverticillatus, Streptomyces griseoviridis, Streptomyces griseus subsp. alpha, Streptomyces griseus subsp. cretosus, Streptomyces griseus subsp. griseus, Streptomyces griseus subsp. solvifaciens, Streptomyces hachijoensis, Streptomyces halstedii, Streptomyces hawaiiensis, Streptomyces heliomycini, Streptomyces helvaticus, Streptomyces herbaricolor, Streptomyces hiroshimensis, Streptomyces hirsutus, Streptomyces humidus, Streptomyces humiferus, Streptomyces hydrogenans, Streptomyces hygroscopicus subsp. angustmyceticus, Streptomyces hygroscopicus subsp. decoyicus, Streptomyces hygroscopicus subsp. glebosus, Streptomyces hygroscopicus subsp. hygroscopicus, Streptomyces hygroscopicus subsp. ossamyceticus, Streptomyces iakyrus, Streptomyces indiaensis, Streptomyces indigoferus, Streptomyces indonesiensis, Streptomyces intermedius, Streptomyces inusitatus, Streptomyces ipomoeae, Streptomyces janthinus, Streptomyces javensis, Streptomyces kanamyceticus, Streptomyces kashmirensis, Streptomyces kasugaensis, Streptomyces katrae, Streptomyces kentuckensis, Streptomyces kifunensis, Streptomyces kishiwadensis, Streptomyces kunmingensis, Streptomyces kurssanovii, Streptomyces labedae, Streptomyces laceyi, Streptomyces ladakanum, Streptomyces lanatus, Streptomyces lateritius, Streptomyces laurentii, Streptomyces lavendofoliae, Streptomyces lavendulae subsp. grasserius: Streptomyces lavendulae subsp. lavendulae, Streptomyces lavenduligriseus, Streptomyces lavendulocolor, Streptomyces levis, Streptomyces libani subsp. libani, Streptomyces libani subsp. rufus, Streptomyces lienomycini, Streptomyces lilacinus, Streptomyces limosus, Streptomyces lincolnensis, Streptomyces lipmanii, Streptomyces litmocidini, Streptomyces lomondensis, Streptomyces longisporoflavus, Streptomyces longispororuber, Streptomyces longisporus, Streptomyces longwoodensis, Streptomyces lucensis, Streptomyces luridiscabiei, Streptomyces luridus, Streptomyces lusitanus, Streptomyces luteireticuli, Streptomyces luteogriseus, Streptomyces luteosporeus, Streptomyces luteoverticillatus, Streptomyces lydicus, Streptomyces macrosporus, Streptomyces malachitofuscus, Streptomyces malachitospinus, Streptomyces malaysiensis, Streptomyces mashuensis, Streptomyces massasporeus, Streptomyces matensis, Streptomyces mauvecolor, Streptomyces mediocidicus, Streptomyces mediolani, Streptomyces megasporus, Streptomyces melanogenes, Streptomyces melanosporofaciens, Streptomyces mexicanus, Streptomyces michiganensis, Streptomyces microflavus, Streptomyces minutiscleroticus, Streptomyces mirabilis, Streptomyces misakiensis, Streptomyces misionensis, Streptomyces mobaraensis, Streptomyces monomycini, Streptomyces morookaensis, Streptomyces murinus, Streptomyces mutabilis, Streptomyces mutomycini, Streptomyces naganishii, Streptomyces narbonensis, Streptomyces nashvillensis, Streptomyces netropsis, Streptomyces neyagawaensis, Streptomyces niger, Streptomyces nigrescens, Streptomyces nigrifaciens, Streptomyces nitrosporeus, Streptomyces niveiciscabiei, Streptomyces niveoruber, Streptomyces niveus, Streptomyces noboritoensis, Streptomyces nodosus, Streptomyces nogalater, Streptomyces nojiriensis, Streptomyces noursei, Streptomyces novaecaesareae, Streptomyces ochraceiscleroticus: Streptomyces odorifer, Streptomyces olivaceiscleroticus, Streptomyces olivaceovihdis, Streptomyces olivaceus, Streptomyces olivochromogenes, Streptomyces olivomycini, Streptomyces olivoreticuli, Streptomyces olivoreticuli subsp. cellulophilus, Streptomyces olivoreticuli subsp. olivoreticuli, Streptomyces olivoverticillatus, Streptomyces olivoviridis, Streptomyces omiyaensis, Streptomyces orinoci, Streptomyces pactum, Streptomyces paracochleatus, Streptomyces paradoxus, Streptomyces parvisporogenes, Streptomyces parvulus, Streptomyces parvus, Streptomyces peucetius, Streptomyces phaeochromogenes, Streptomyces phaeofaciens, Streptomyces phaeopurpureus, Streptomyces phaeoviridis, Streptomyces phosalacineus, Streptomyces pilosus, Streptomyces platensis, Streptomyces plicatus, Streptomyces pluricolorescens, Streptomyces polychromogenes, Streptomyces poonensis, Streptomyces praecox, Streptomyces prasinopilosus, Streptomyces prasinosporus, Streptomyces prasinus, Streptomyces prunicolor, Streptomyces psammoticus, Streptomyces pseudoechinosporeus, Streptomyces pseudogriseolus, Streptomyces pseudovenezuelae, Streptomyces pulveraceus, Streptomyces puniceus, Streptomyces puniciscabiei, Streptomyces purpeofuscus, Streptomyces purpurascens, Streptomyces purpureus, Streptomyces purpurogeneiscleroticus, Streptomyces racemochromogenes, Streptomyces rameus, Streptomyces ramulosus, Streptomyces rangoonensis, Streptomyces recifensis, Streptomyces rectiverticillatus, Streptomyces rectiviolaceus, Streptomyces regensis, Streptomyces resistomycificus, Streptomyces reticuliscabiei, Streptomyces rhizosphaericus, Streptomyces rimosus subsp. paromomycinus, Streptomyces rimosus subsp. rimosus, Streptomyces rishiriensis, Streptomyces rochei, Streptomyces roseiscleroticus, Streptomyces roseodiastaticus, Streptomyces roseoflavus, Streptomyces roseofulvus, Streptomyces roseolilacinus, Streptomyces roseolus, Streptomyces roseosporus, Streptomyces roseoverticillatus, Streptomyces roseoviolaceus, Streptomyces roseoviridis, Streptomyces rubber, Streptomyces rubiginosohelvolus, Streptomyces rubiginosus, Streptomyces rubrogriseus, Streptomyces rutgersensis subsp. castelarensis, Streptomyces rutgersensis subsp. rutgersensis, Streptomyces salmonis, Streptomyces sampsonii, Streptomyces sanglieri, Streptomyces sannanensis, Streptomyces sapporonensis, Streptomyces scabiei, Streptomyces sclerotialus, Streptomyces scopiformis, Streptomyces seoulensis, Streptomyces septatus, Streptomyces setae, Streptomyces setonii, Streptomyces showdoensis, Streptomyces sindenensis, Streptomyces sioyaensis, Streptomyces somaliensis, Streptomyces sparsogenes, Streptomyces spectabilis, Streptomyces speibonae, Streptomyces speleomycini, Streptomyces spheroids, Streptomyces spinoverrucosus, Streptomyces spiralis, Streptomyces spiroverticillatus, Streptomyces spitsbergensis, Streptomyces sporocinereus, Streptomyces sporoclivatus, Streptomyces spororaveus, Streptomyces sporoverrucosus, Streptomyces stelliscabiei, Streptomyces stramineus, Streptomyces subrutilus, Streptomyces sulfonofaciens, Streptomyces sulphurous, Streptomyces syringium, Streptomyces tanashiensis, Streptomyces tauricus, Streptomyces tendae, Streptomyces termitum, Streptomyces thermoalcalitolerans, Streptomyces thermoautotrophicus, Streptomyces thermocarboxydovorans, Streptomyces thermocarboxydus, Streptomyces thermocoprophilus, Streptomyces thermodiastaticus, Streptomyces thermogriseus, Streptomyces thermolineatus, Streptomyces thermonitrificans, Streptomyces thermospinosisporus, Streptomyces thermoviolaceus subsp. apingens, Streptomyces thermoviolaceus subsp. thermoviolaceus, Streptomyces thermovulgaris, Streptomyces thioluteus, Streptomyces torulosus, Streptomyces toxytricini, Streptomyces tricolor, Streptomyces tubercidicus, Streptomyces tuirus, Streptomyces turgidiscabies, Streptomyces umbrinus, Streptomyces variabilis, Streptomyces variegates, Streptomyces varsoviensis, Streptomyces vastus, Streptomyces venezuelae, Streptomyces vinaceus, Streptomyces vinaceusdrappus, Streptomyces violaceochromogenes, Streptomyces violaceolatus, Streptomyces violaceorectus, Streptomyces violaceoruber, Streptomyces violaceorubidus, Streptomyces violaceus, Streptomyces violaceusniger, Streptomyces violatus, Streptomyces violascens, Streptomyces violatus, Streptomyces violens, Streptomyces vixens, Streptomyces virginiae, Streptomyces viridiflavus, Streptomyces viridiviolaceus, Streptomyces viridobrunneus, Streptomyces viridochromogenes, Streptomyces viridodiastaticus, Streptomyces viridosporus, Streptomyces vitaminophileus, Streptomyces vitaminophilus, Streptomyces wedmorensis, Streptomyces werraensis, Streptomyces willmorei, Streptomyces xanthochromogenes, Streptomyces xanthocidicus, Streptomyces xantholiticus, Streptomyces xanthophaeus, Streptomyces yatensis, Streptomyces yerevanensis, Streptomyces yogyakartensis, Streptomyces yokosukanensis, Streptomyces yunnanensis, Streptomyces zaomyceticus, Streptoverticillium abikoense, Streptoverticillium albireticuli, Streptoverticillium alboverticillatum, Streptoverticillium album, Streptoverticillium ardum, Streptoverticillium aureoversale, Streptoverticillium aureoversile, Streptoverticillium baldaccii, Streptoverticillium biverticillatum, Streptoverticillium blastmyceticum, Streptoverticillium cinnamoneum subsp. albosporum, Streptomyces cinnamoneus subsp. albosporus, Streptoverticillium cinnamoneum subsp. cinnamoneum, Streptoverticillium cinnamoneum subsp. lanosum, Streptoverticillium cinnamoneum subsp. sparsum, Streptoverticillium distallicum, Streptoverticillium ehimense, Streptoverticillium eurocidicum, Streptoverticillium fen/ens subsp. fervens, Streptoverticillium fervens subsp. melrosporus, Streptoverticillium flavopersicum, Streptoverticillium griseocarneum, Streptoverticillium griseoverticillatum, Streptoverticillium hachijoense, Streptoverticillium hiroshimense, Streptoverticillium kashmirense, Streptoverticillium kentuckense, Streptoverticillium kishiwadense, Streptoverticillium ladakanum, Streptoverticillium lavenduligriseum, Streptoverticillium lilacinum, Streptoverticillium luteoverticillatum, Streptoverticillium mashuense, Streptoverticillium mobaraense, Streptoverticillium morookaense, Streptoverticillium netropsis, Streptoverticillium olivomycini, Streptomyces olivomycini, Streptoverticillium olivoreticuli subsp. cellulophilum, Streptoverticillium olivoreticuli subsp. olivoreticuli, Streptoverticillium olivoreticulum, Streptoverticillium olivoreticulum subsp. cellulophilum, Streptoverticillium olivoverticillatum, Streptoverticillium orinoci, Streptoverticillium parvisporogenes, Streptoverticillium parvisporogenum, Streptoverticillium rectiverticillatum, Streptoverticillium reticulum subsp. protomycicum, Streptoverticillium roseoverticillatum, Streptoverticillium salmonis, Streptoverticillium sapporonense, Streptoverticillium septatum, Streptoverticillium syringium, Streptoverticillium thioluteum, Streptoverticillium verticillium subsp. quantum, Streptoverticillium verticillium subsp. tsukushiense or Streptoverticillium viridoflavum.

### Agricultural Products

As described herein, an agricultural product can be a product of an industrial agricultural process, a product of farming, a byproduct or waste product of agricultural process. Without bound by theory, any organism described herein can be evolutionarily adapted to grow on any agricultural product, either exclusively or inclusively on one or more agricultural products described herein. An organism can be adapted to grow and utilize carbon source from two or more agricultural products. Examples of agricultural products include, but are not limited to, stillage, stover, bagasse, pennycress seeds, soy stems, soy leaves, soy pods, soy molasses, soy flakes, peels, bean cake, seeds, seed cake, wheat straw, switchgrass or wood chips. Other agricultural products include various plant material such as grains such as maize (corn, e.g., whole ground corn), sorghum (milo), barley, wheat, rye, rice, and millet; and starchy root crops, tubers, or roots such as sweet potato or cassaya, corn fiber, or corn cobs. In one embodiment, agricultural product includes municipal waste, wood, plant material, plant material extract, a natural or synthetic polymer, or a combination thereof. In another embodiment, agricultural products inlcude plant matter such as woody plant matter, such as wood chip, non-woody plant matter, cellulosic material, lignocellulosic material, hemicellulosic material, carbohydrates, pectin, starch, inulin, fructans, glucans, corn, corn stover, sugar cane, grasses, switch grass, or bamboo.

Compositions of Agricultural Products

Various compositions of agricultural products can be a carbon/nitrogen source for the growth of an EMO. For example, an agricultural product can include lignocellulose, cellulose, hemicellulose, lignin, and other proteins and biological molecules such as a vitamin.

A lignocellulose is any polymeric composite formed by plants. In one embodiment, a lignnocellulose is a plant structural material. In another embodiment, a lignocellulose comprises one or more lignin, one or more cellulose, and one or more hemicellulose.

A cellulose is a glucose polymer. In one embodiment, a cellulose is a glucose dimer In another embodiment, a cellulose is cellubiose. In another embodiment, a cellulose is two or more glucose molecules linked by beta-1,4, glycosidic bonds.

A hemicellulose is a branched polymer of five (C5) or six (C6) carbon sugars. In one embodiment, a hemicellulose comprises a xylose. In another embodiment, a hemicellulose comprises an arabinose. In another embodiment, a hemicellulose comprises a mannose. In another embodiment, a hemicellulose comprises a rhamnose. In another embodiment, a hemicellulose comprises galactose. In another embodiment, a hemicellulose comprises glucose. In another embodiment, a hemicellulose is amorphous hemicellulose. In one embodiment, a hemicellulose is hydrolyzed by an EMO. In another embodiment, EMO produces xylose by hydrolyzing a hemicellulose.

A lignin is plant phlyphenolic molecule. In one embodiment, an EMO is adapted to break down a lignin. In another embodiment, an EMO adapted to break down a lignin is a fungal species. In another embodiment, the fungal species is Trichoderma reesei. In another embodiment, the fungal species is Phanerochaete chrysosporium. In another embodiment, the fungal species converts lignin to fungal biomass. In another embodiment, the fungal biomass is boiled and used to grow heterotrophic algae.

Ash is produced after dry oxidation of agricultural products, containing minerals such as aluminum, calcium magnesium, potassium or sodium. In one embodiment, ash is used to supplement the growth of an EMO.

Other compounds obtainable from agricultural products include fats, resins, fatty acids, phenolics, or phytosterols. Other compounds of interests are any agricultural by-products or any industrial organic waste stream comprising carbohydrates and proteins. These materials are preferably used as raw material or optionally further processed to support a culture of an EMO.

Stillage

Provided herein are methods for producing fatty acids, hydro-carbons or fermentation product for use in biofuel or chemicals production from stillage. The stillage can be from any type of grain, such as corn. In one embodiment, the stillage is an oilseed stillage.

Stillage produced from a plant can be a useful source for growing an EMO. In one ebodiment a plant useful for culture with a EMO is from the class of Liliopsida, the family of Arecaceae: Oil Palm (Elaeis guineensis), Macauba Palm (Acrocomia aculeate), Buriti Palm (Mauritia flexuosa), Coconut (Cocos nucifera), Babassu Palm (Orbignya martiana), Piassaya (Attalea funifera), Palm (Erythea salvadorensis); and the family of Poaceae: Rice (Oriza sativa L), Oat (Avena sativa), Corn (Zea cans); in the class of Magnoliopsida, the family of Fabaceae: Peanut (Arachis hypogaea), Soybean (Glycine max), Lupine (Lupinus albus); the Euphorbiaceae family: Jatropha (Jatropha curcas), Castor Bean (Ricinus communis), Gopher Plant (Euphorbia lathyris), Tung Oil Tree (Aleurites fordii), Euphorbia (Euphorbia lagascae), Rubber Seed (Hevea brasiliensis); the Brassicaceae Family: Crambe (Crambe abyssinica), Rapeseed (Brassica napus), Camelina (Camelina sativa), Mustard (Brassica alba); the Family Asteraceae: Sunflower (Helianthus annuus), Safflower (Carthamus tinctorius), Calendula (Calendula officinalis); the Family Malvaceae: Cotton (Gossypium hirsutum), Kenaf (Hibiscus cannabinus); the Family Cucurbitaceae: Buffalo Gourd (Cucurbita foetidissima), Pumpkin Seed (Cucurbita pepo); and in the class of Magnoliopsida: Family Anacardiaceae: Cashew Nut (Anacardium occidentale) Family Apiaceae: Coriander (Coriandrum sativum), Family Betulaceae: Hazelnut (Corylus avellana), Family Cannabaceae: Hemp (Cannabis sativa), Family Caryocaraceae: Pequi (Caryocar brasiliense), Family Chrysobalanaceae: Oiticia (Licania rigida), Family Clusiaceae: Bacuri (Platonia insignis), Family Juglandaceae: Pecan (Carya illinoinensis), Family Lauraceae: Avocado (Persea americana), Family Lecythidaceae: Brazil Nut (Bertholletia excelsa) Family Linaceae: Linseed (Linum usitatissimum), Family Oleaceae: Olive Tree (Olea europaea), Family Papaveraceae: Opium Poppy (Papaver somniferum), Family Pedaliaceae: Sesame (Sesamum indicum), Family Proteaceae: Macadamia Nut (Macadamia terniflora) Family Rubiaceae: Coffee (Coffea arabica), Family Simmondsiaceae: Jojoba (Simmondsia chinensis), or Family Sterculiaceae: Cocoa (Theobroma cacao)

In one embodiment, a stillage is useful for producing fatty acids, biofuel, biodiesel, or other end products by incubating the stillage with one or more EMOs. The type of grains or stillage includes, but is not limited to, Distillers Dried Solubles (DDS), Distillers Dried Grains (DDG), Condensed Distillers Solubles (or condensed distillers grains with solubles) (CDS), Distillers Wet Grains (DWG), or Distillers Dried Grains with Solubles (DDGS). In one embodiment, the stillage comprises wet corn distillers grains with solubles (WDGS). In another embodiment, the stillage comprises corn distillers grains (DG).

Composition of a stillage varies according to each plant species, comprised of a small portion of oil remaining after the seed processing, a proportion of carbohydrates, a proportion of ashes, and, a proportion of proteins. In one embodiment, a composition of WDGS and DDGS as shown in Tables 1, 2, or both, is used. In one embodiment, an organism evolved to grow on a common component of a stillage, such as cellulose in the fiber, can be further evolved to grow robustly on a particular type of stillage. In one embodiment, a microorganism evolved to utilize hemicellulose is further evolved to grow on stillage from Zea cans.

TABLE 1
Nutrient value of selected wet corn milling co-products.

| | Dry Corn | Corn | Wet Corn | Condensed |
|---|---|---|---|---|
| | Gluten | Gluten | Gluten | Steep Water |
| | Feed | Meal | Feed | Solubles |
| Dry Matter | 90 | 90 | 42-44 | 50 |
| Protein, % | 20 | 66 | 17-21 | 35 |
| Fat, % | 2.8 | 2.2 | 3-4 | 3 |
| Fiber, % | 11.1 | 3.3 | 3.5 | — |
| ADF, % | 37.6 | 14 | 12-14 | 6-1 |
| NDF, % | 12.4 | 5 | 38.48 | 1.5 |
| TDN, % | 80 | 86 | 80 | 91 |
| NEm, mcal/lb | 0.96 | 1 | 0.9-0.97 | 1 |
| NEg, mcal/lb | 0.59 | 0.69 | 0.62-0.67 | .72 |
| Ca, % | 0.055 | 0.08 | 0.03-0.1 | 0.07 |
| P, % | 1.1 | 0.53 | 0.5-1 | 2 |
| K, % | 1.6 | 0.22 | 1 | 3 |
| Mg, % | 0.5 | 0.088 | 0.3 | 0.9 |
| Na, % | 0.16 | 0.066 | 0.1 | 0.4-0.5 |
| S, % | 0.33 | 0.72 | 0.4-0.5 | 1.8-2 |
| Fe, ppm | 178 | 313 | 80-110 | 127-178 |
| Cu, ppm | 5.5 | 27 | 5-7 | 8 |
| Zn, ppm | 83.3 | 34 | 46-64 | 113-164 |
| Mn, ppm | 23.3 | 7.8 | 0.3 | 42-50 |

TABLE 2
Nutrient value of selected dry corn milling coproducts.

| | Distilled | Cond. | Wet | Modified |
|---|---|---|---|---|
| | Dried Grains | Distillers | Distillers | Distillers |
| | Solubles | Solubles | Grains | Grains |
| Dry Matter | 89 | 25-45 | 31-36 | 46-51 |
| Protein, % | 31 | 14-23 | 32-36 | 26-32 |
| Fat, % | 11 | 15-24 | 9-12 | 11-16 |
| Fiber, % | 7.2 | — | — | 5-15 |
| ADF, % | 12 | — | 10-12 | 11-18 |
| NDF, % | 45 | — | 30-50 | 35-50 |
| TDN, % | 87 | 95-120 | 90-110 | 90-110 |
| NEm, mcal/lb | 0.99 | 1.05-1.15 | 0.9-1.1 | .9-1.1 |
| NEg, mcal/lb | 0.68 | 0.85-0.93 | 0.7-0.8 | .7-.8 |
| Ca, % | .07 | 0.03-0.17 | 0.02-.05 | <.10 |
| P, % | .77 | 1.3-1.5 | 0.4-0.5 | .85-1.4 |
| K, % | 1.00 | 1.75-2.25 | 0.5-1 | 1-1.5 |
| Mg, % | .30 | 0.65-0.9 | 0.2-0.3 | .3-.5 |
| Na, % | .18 | .2-.4 | 0.1-0.2 | .2-.3 |
| S, % | .68 | .9-1.4 | .4-.6 | .4-1.2 |
| Fe, ppm | 127 | 90-120 | 70-180 | 67-130 |
| Cu, ppm | 6 | 6-7 | 6-7 | 5-7 |
| Zn, ppm | 62 | 100-140 | 40-80 | 62 |
| Mn, ppm | 19 | 30-35 | 8-16 | 27 |

Fermentation byproducts from fermentain corn, wheat, sorghum or barley are useful for growing an EMO. A fermentation byproduct includes, but is not limited to, thin stillage or DG. In one embodiment, a thin stillage or DG comprises ash. In another embodiment, a thin stillage or DG comprises ether extract. In another embodiment, a thin stillage or DG comprises neutral detergent fiber. In another embodiment, a thin stillage or DG comprises acid detergent fiber. In another embodiment, a thin stillage or DG comprises crude protein. In another embodiment, a thin stillage or DG comprises starch. In another embodiment, a thin stillage or DG comprises carbohydrates. In another embodiment, a thin stillage or DG comprises non-structural carbohydrates. In another embodiment, a DG comprises about 50% or more carbohydrates. In some embodiments, a DG comprises about 10%, 20%, 30%, 40% or more carbohydrates. In another embodiment, a thin stillage comprises about 30% or more carbohydrates. In some embodiments, a thin stillage comprises about 5%, 10%, 15%, 20%, 25% or more carbohydrates.

In one embodiment, DDGS are used to grow an EMO. In one embodiment, DDGS comprises, by weight, about 5%, 6%, 7%, 8%, 9%, 10%, 11%, 12%, 13%, 14% or 15% of moisture. In another embodiment, DDGS comprises, by weight, about more than 70%, 75%, 80%, or 85% of dry matter content. In another embodiment, DDGS comprises crude protein. In another embodiment, DDGS comprises crude fiber. In another embodiment, DDGS comprises starch. In another embodiment, DDGS comprises acid detergent fiber. In another embodiment, DDGS comprises ash. In another embodiment, DDGS comprises amino acids.

In one embodiment a whole stillage is used to grow an EMO. In one embodiment, a whole stillage comprises about, by weight, more than 70%, 75%, 80%, or 85% of moisture. In one embodiment, a whole stillage comprises crude vegetable oil. In another embodiment, a whole stillage comprises carbohydrates including starch, cellulose, hemicellulose, xylose, galactose, arabinose, and lignin. In another embodiment, a whole stillage comprises crude proteins and ash.

Seed Cake

Some species of Euphorbiaceae (jatropha, castor bean, gopher plant or euphorbia) produce high yield of oil. Often plants of the family Euphorbiaceae are toxic, leaving their seed cake or the biomass resulting of their culture unusable as animal feed or as a raw material for the food industry. Because of their toxicity, their cropping or pressed bean residues are either poorly valued as field fertilizer or disposed.

Jatropha curcas and castor beans can be used as oilseed crops for the production of biofuels. Their seeds contain high levels of oil (35% and 50%, respectively) that can be transesterified into high quality biodiesel or other hydrocarbon-based fuels. The oil is obtained by pressing the seeds and seed cake is a by-product of this process. For some oilseeds, the seed cake is a valuable co-product that contains proteins, carbohydrates and other nutrients. This seed cake can be sold on the animal and human feed market. For Jatropha and castor beans, the seed cake contains toxic compounds (e.g., ricin) that must be neutralized in order to be used as feed. Even with toxin neutralization, consumers are reluctant to use the seed cake as feed and, consequently, this by-product is commonly wasted or returned to the field as fertilizer.

Described herein are related to methods for producing fatty acids, hydrocarbons or fermentation products from seed cake, such as oilseed seed cake. In one embodiment, fatty acids are produced from oilseed seed cake. In another embodiment, a biofuel is produced from said fatty acids. Hydrolyzing a mixture of at least one protein issued from such seed cake, can be done using one of commonly known techniques of hydrolysis, including heat exposure, steam exposure, acid, alkaline or enzymatic method that results into the breakdown of proteins into various amino acids. In one embodiment, a seed cake is Euphorbiaceae seed cake. In one embodiment, a seed cake is Jatropha seed cake. In another embodiment, a seed cake is castor seed cake. In another embodiment, a seed cake or biomass is from gopher plant. In another embodiment, a seed cake or biomass is from a euphorbia plant.

In one embodiment, a method of producing fatty acids for biofuel, biodiesel or other useful end-products from the bioconversion of cellulosic portion of been cake or biomass can be achieved. In one embodiment, conversion of cellulosic portion to valuable chemicals is achieved by methods disclosed in U.S. Provisional Application No. 61/058,096, which is incorporated herein in its entirety by reference.

In one embodiment, a process produces fatty acids, hydro-carbons, or other end products from a seed cake. In another embodiment, the seed cake is processed either alone or mixed with other carbon or nitrogen source compound.

In one embodiment, an evolutionarily modified microorganism is inoculated into a mixture of seed cake in which the mixture is a mixture of cellulosic and non-cellulosic material. In another embodiment, the microorganism metabolizes at least one of amino acids in the mixture as carbon or nitrogen source. In another embodiment, under conditions that microorganisms metabolizing at least one amino acid, the microorganism produces one or more fatty acids, one or more hydrocarbons or one or more fermentation products. In another embodiment, the one or more fatty acids, one or more hydrocarbons, or one or more fermentation products are recovered from the culture of microorganism with the mixture. In another embodiment, the microorganism is evolutionarily modified to better tolerate any of the components included in the mixture.

Stover

In one embodiment, an agricultural product is stover. In one embodiment, a stover is a corn stover. In another embodiment, a stover is a sorghum stover. In another embodiment, a stover is a soybean stover. In another embodiment, a stover comprises cellulosic or lignocellulosic materials obtained from plant biomass. In another embodiment, the biomass comprises one or more of corn steep solids, corn steep liquor, malt syrup, xylan, cellulose, hemicellulose, fructose, glucose, galactose, mannose, rhamnose, or xylose. In another embodiment, corn stover comprises glucan. In another embodiment, corn stover comprises xylose and arabinose. In another embodiment, corn stover comprises ash and acetate. In another embodiment, corn stover comprises more than about 25%, 28%, 30%, 32%, 35%, 37% of cellulose by dry weight. In another embodiment, cellulose in corn stover comprises glucan.

Bagasse

A bagasse obtained from plant material can be useful for growing an EMO. Plaint material includes, but is not limited to, sugarcane or sorghum. An industrial agricultural process, such as sugar-production process produces large amount of bagasse from processing, for example, sugarcane. Fibrous material in bagasse contains carbohydrate, hemicellulose, or polysaccharides that can be used for growing an EMO. The fibrous material can be either processed to extract polysaccharides. Processing can involve physical or mechanical steps, such as shearing, pressing, tearing, freeze-drying, filtering, dewatering or chemical steps, such as dewaxing, or enzymatical steps. Chemical steps include treating with toluene, NaOH, NaCl or other treatment affecting the pH of the bagasse extracts. In one embodiment, a bagasse extract comprises rhamnose. In another embodiment, a bagasse extract comprises arabinose. In another embodiment, a bagasse extract comprises galactose. In another embodiment, a bagasse extract comprises glucose. In another embodiment, a bagasse extract comprises mannose. In another embodiment, a bagasse extract comprises xylose. In another embodiment, a bagasse extract comprises uronic acid. In another embodiment, a bagasse extract comprises glucose, mannose, xylose, uronic acid, galactose, arabinose, and rhamnose. In another embodiment, a bagasse extract comprises more than, by weight, about 20%, 30%, 40%, 45%, 50%, 51%, 52%, 53%, 54%, 55%, 56%, 57%, 58% 59%, or 60% of glucose. In another embodiment, a dry matter of bagasse extract comprises more than about 30%, 40%, or 50% of total weight. In another embodiment, a bagasse extract comprises protein, netral fiber and acid fiber. In another embodiment, a bagasse extract comprises crude protein, fat and ash. In another embodiment, a bagasse extract comprises minerals such as potassium, magnesium, manganese, calcium, iron, phosphate, zinc, cadmium, copper, and sodium. Compositions of bagasse or bagasse extract described in the following literatures are incorporated herein in their entirety: El-Sayed et al., Resources, Conservation and Recycling, 12:195-200, 1994; Okano et al., Animal Science Journal, 77:308-313, 2006; Carvalho et al., Arq. Bras. Med. Vet. Zootec., 61:1346-1352, 2009; Peng et al., Food Research International, 43:683-693, 2010.

Peels

Vegetable or fruit peels are useful source for growing an EMO. Industrial processing of furits, such as canned fruits produce large amount of peels. Peels contain cellulosic material upon which a microorganism can be modified, by methods described herein, to grow either exclusively or inclusively on the cellulosic material obtained from peels. A fruit can be any fruit either suitable for human or animal consumption. A fruit can by any product of a plant containing seed. In one embodiment, a fruit peel is apple peel. In another embodiment, an apple peel is Budimka apple. In another embodiment, a fruit peel is from apple pomace. In another embodiment, a fruit peel is a citrus peel. In another embodiment, a fruit peel is potato peel.

In one embodiment, apple peel comprises reducing sugars. In another embodiment, apple peel comprises Klason lignin. In another embodiment, apple peel comprises soluble klason lignin. In another embodiment, apple peel comprises pectin. In another embodiment, apple peel comprises nucleic acids. In another embodiment, apple peel further comprises ash, phosphorus, calcium and potassium. In one embodiment, pectins from apple peel comprises oligogalactouronic acid. In another embodiment, the oligogalactouronic acid comprises one or more dimers. In another embodiment, the oligogalactouronic acid comprises one or more trimers. In some embodiments, the oligogalactouronic acid comprises one or more tetramer, pentamer, hexamer, heptamer or octamer.

In one embodiment, an apple peel extract comprises rhamnose. In another embodiment, an apple peel extract comprises arabinose. In another embodiment, an apple peel contains rhamnose, arabinose, xylose, mannose, galactose, glucose. Compositions of apple peel or apple peel extract described in the following literatures are incorporated herein in their entirety: Massiot et al., Carbohydrate Polymers, 25:145-154, 1994; Nikolic et al., Journal of the Serbian Chemical Society, 73:157-167, 2008; Villas-Boas et al., World Journal of Microbiology and Biotechnology, 19:461-467, 2003.

In one embodiment, a citrus peel is a peel from orange. In another embodiment, a citrus peel is a peel from Mandarin orange. In another embodiment, a citrus peel comprises ash, sugars, fat, protein, pectin, lignin, cellulose and hemicellulose. In another embodiment, a citrus peel comprises crude protein, lactic acid, acetic acid, and acid or neutral detergent fibers. In another embodiment, a citrus peel comprises propionic acid. In another embodiment, a citrus peel comprises isobutyric acid. Compositions of citrus peel or citrus peel extract described in the following literatures are incorporated herein in their entirety: Lopez et al., Critical Reviews in Biotechnology, 30:63-69, 2010; Boluda-Aguilar et al., Bioresource Technology, 101:3506-3513, 2010.

In one embodiment, a potato peel comprises pectin, cellulose, starch, and ash. In another embodiment, a potato peel comprises volatile solids, alcohol-insoluble solids, and reducing sugars. In another embodiment, the pH of a potato peel extract is about 6. In some other embodiments, the pH of a potato peel extract is about 4.0, 4.2, 4.5, 5.0, 5.2, 5.5, 6.2, 6.5, 6.8 or 7.0. In another embodiment, a potato peel comprises element metal such as iron, silicon, aluminum or titanium. In another embodiment, the moisture of potato peel comprises less than 10% by weight. In another embodiment, a potato peel comprises more than about 50% cellulose by weight. In another embodiment, a potato peel comprises more than about 11% hemicellulose by weight. In another embodiment, a potato peel comprises more than about 14% lignin by weight. In another embodiment, a potato peel comprises more than 5% ash by weight. In another embodiment, for 100 g of potato peel, at least 50 g of glucose is produced. In another embodiment, at least 2.5 g of arabinose is produced from 100 g of potato peel. In another embodiment, nitrogen content in potato peel comprises at least 1.5% of dry matter. In another embodiment, carbon content of potato peel comprises at least 40% of dray matter. Compositions of potato peel or potato peel extract described in the following literatures are incorporated herein in their entirety: Lenihan et al., Chemical Engineering Journal, 156:395-403, 2010; Mahmood et al., Enzyme and Microbiol Technology, 22:130-137, 1998; Kryvoruchko et al., Biomass and Bioenergy, 33:620-627, 2009

Sugar Beet

Sugar beet can be used to produce sugar beet pulp silage (SBP) or sugar beet tail silage (SBT). SBT is the lowest part of the sugar been that is cut off from the beet during the cleaning process. Produced as stillage, they can be a convenient carbon or nitrogen source available year-round. Both SBP and SBT portions contain sugars that can be extracted and fermented to produce ethanol. In addition to anaerobic digestion of SBP or SBT, these materials can be utilized to grow an EMO. In one embodiment, an EMO adapted to grow on SBP or SBT is an alga.

In one embodiment, nitrogen content of SBP comprises at least 1.2% of dry matter. In another embodiment, nitrogen content of SBT comprises at least 2% of dry matter. In another embodiment, carbon content of SBP comprises at least 45% of dry matter. In another embodiment, carbon content of SBP comprises at least 32% of dry matter. Compositions of SBP or SBT described in the following literatures are incorporated herein in their entirety: Kryvoruchko et al., Biomass and Bioenergy, 33:620-627, 2009. In another embodiement, a method disclosed herein can be applied to metabolize any type of agricultural by-product or any industrial organic waste stream comprising carbohydrates and proteins.

End Products

An organism, method or composition disclosed herein can be used to produce one or more end products, such as a biofuel, or a product used to generate a biofuel. Furthermore, an organism or method disclosed herein can be used to produce other valuable chemicals. In one embodiment, various fermentative end-products can be produced through further saccharification and fermentation using an EMO on stillage. In one embodiment, more than one type or species of organism is used to produce an end product. For example, a co-culture of a fungi or bacteria with algae is used to generate one or more end products. The one or more organisms in a co-culture can be an EMO.

In one embodiment, the end-product is methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-) hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid or formic acid. In another embodiment the end-product is a lipid or a fatty acid.

In one embodiment, an organism disclosed herein can be used to produce a hydrocarbon-rich molecule, e.g. a terpene, such as using a method disclosed herein. A terpene (classified by the number of isoprene units) can be a hemiterpene, monoterpene, sesquiterpene, diterpene, triterpene, or tetraterpene. In one embodiment, the terpene is a terpenoid (aka isoprenoid), such as a steroid or carotenoid. Subclasses of carotenoids include carotenes and xanthophylls. In specific embodiments, a fuel product is limonene, 1,8-cineole, α-pinene, camphene, (+)-sabinene, myrcene, abietadiene, taxadiene, farnesyl pyrophosphate, amorphadiene, (E)-α-bisabolene, beta carotene, alpha carotene, lycopene, fusicoccadiene or diapophytoene. Some of these terpenes are pure hydrocarbons (e.g. limonene) and others are hydrocarbon derivatives (e.g. cineole).

In one embodiment, an organism disclosed herein can be used to produce an isoprenoid, such as by growing an organism, such as an alga. An isoprenoid can be produced from an algal biosynthetic pathway, such as through a synthase (e.g. a C5, C10, C15, C20, C30, C40 synthase, or combination thereof). In one embodiment, isoprenoids that can be produced include, but are not limited to, botryococcene, -caryophyllene, germacrene, 8-epicedrol, valencene, (+)-1-cadinene, germacrene C, (E)-1-farnesene, casbene, vetispiradiene, 5-epi-aristolochene, aristolchene, -humulene, (E,E)-1-farnesene, (−)-1-pinene, -terpinene, limonen, linalool, geranyl diphosphate, levopimaradiene, isopimaradiene, (E)-1-bisabolene, copalyl pyrophosphate, kaurene, longifolene, -humulene, -selinene, -phellandrene, terpinolene, (+)-3-carene, syn-copalyl diphosphate, -terpineol, syn-pimara-7,15-diene, ent-sandaaracopimaradiene, E-1-ocimene, S-linalool, geraniol, -terpinene, linalool, E-1-ocimene, epi-cedrol, -zingiberene, guaiadiene, cascarilladiene, cis-muuroladiene, aphidicolan-16b-ol, elizabethatriene, sandalol, patchoulol, zinzanol, cedrol, scareol, or copalol.

Mycolic acids are naturally produced by certain types of bacteria. These bacteria can be evolutionarily modified to robustly produce mycolic acids or their related compounds. Examples of bacteria adaptable for producing mycolic acid include Corynobacterium, Nocardia, and Mycobacterium. Corynobacterium produced mycolic acids are referred to as Corynnomycolic acids, which typically contain 22-36 carbon atoms. Nocardomycolic acids typically contain 44-60 carbon atoms and are produced by Nocardia bacterium. Mycolic acids isolated from Mycobacterium are also called eumycolic acids. Mycolic acids produced by Mycobacterium generally have 60-90 carbon atoms. A description of the various forms of mycolic acids found in Mycobacterium may be found in a review by Minnikin D E et al. (Arch Microbiol 1984, 139, 225), incorporated herein by reference.

In one embodiment, the methods described herein produce mycolic acid. Mycolic acids as used herein includes methoxymycolic acids, ketomycolic acids, epoxymycolic acids, or mycolic acid wax esters containing a double bond or a cyclopropane ring with an internal ester group. In one embodiment, mycolic acid is produced by growing Nocardia bacterium on stillage.

Mycobacterium smegmatis is a non-pathogenic, is a fast-growing and non-fastiduous bacterium (L. G. Wayne, Kubica, and G. P.: The Mycobacteria. In: Holt, J. G., Sneath, P. H., Mair, N. S, Sharpe, M. E. (Eds.). Bergey's Manual of Systematic Bacteriology Vol. 2. The Williams Wilkins Co.; Baltimore, Md.: 1435-1457, 1986, incorporated herein by reference). In one embodiment, mycolic acid is produced by evolutionarily modified Mycobacterium smegmatis.

Biofuels

The one or more end products produced by an organism, method, or composition disclosed herein, such as those described above, can be a biofuel or a product used to generate or produce a biofuel. The biofuel can be used in a combustor such as a boiler, kiln, dryer or furnace. Other examples of combustors are internal combustion engines such as vehicle engines or generators, including gasoline engines, diesel engines, jet engines, and others. Biofuels can also be used to produce plastics, resins, fibers, elastomers, lubricants, and gels.

The biofuel can include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but are not limited to, nitrogen, oxygen, sulfur, and phosphorus. Some products are hydrocarbon-rich, wherein at least 50%, 60%, 70%, 80%, 90%, 95, 99% of the product by weight is made up carbon and hydrogen. In an embodiment, a product is 100% by weight carbon and hydrogen atoms. In some embodiments, the products comprise terpenes. In other embodiments, the products comprise fatty acids or fatty acid methyl esters.

A biofuel can be a precursor or product conventionally derived from crude oil, or petroleum, such as, liquid petroleum gas, naptha (ligroin), gasoline, kerosene, diesel, lubricating oil, heavy gas, coke, asphalt, tar, or waxes. In one embodiment, a biofuel can include small alkanes (for example, 1 to approximately 4 carbons) such as methane, ethane, propane, or butane, which can be used for heating (such as in cooking) or making plastics. A biofuel can also include molecules with a carbon backbone of approximately 5 to approximately 9 carbon atoms, such as naptha or ligroin, or their precursors. Other biofuels can be about 5 to about 12 carbon atoms or cycloalkanes used as gasoline or motor filet. Molecules and aromatics of approximately 10 to approximately 18 carbons, such as kerosene, or its precursors, can also be biofuels. Biofuels can also include molecules, or their precursors, with more than 12 carbons, such as used for lubricating oil. Other biofuels include heavy gas or fuel oil, or their precursors, typically containing alkanes, cycloalkanes, and aromatics of approximately 20 to approximately 70 carbons. Biofuels also include other residuals from crude oil, such as coke, asphalt, tar, and waxes, generally containing multiple rings with about 70 or more carbons, and their precursors. Biofuels also include alcohols, such as ethanol, methanol, butaonl, or propanol.

The various biofuels can be further refined to a final product for an end user by a number of processes. Refining can occur by fractional distillation. For example, a mixture of fuel products, such as a mix of different hydrocarbons with different various chain lengths can be separated into various components by fractional distillation.

Refining can also include any one or more of the following steps: cracking, unifying, or altering the biofuel. Large fuel products, such as large hydrocarbons (for example C 10), can be broken down into smaller fragments by cracking. Cracking can be performed by heat or high pressure, such as by steam, visbreaking, or coking. Biofuels can also be refined by visbreaking, for example reducing the viscosity of heavy oils. Refining can also include coking, wherein a heavy, almost pure carbon residue is produced. Cracking can also be performed by catalytic means to enhance the rate of the cracking reaction by using catalysts such as, a zeolite, aluminum hydrosilicate, bauxite, or silica-alumina Catalysis can be by fluid catalytic cracking, whereby a hot catalyst, such as zeolite, is used to catalyze cracking reactions. Catalysis can also be performed by hydrocracking, where lower temperatures are generally used in comparison to fluid catalytic cracking. Hydrocracking typically occurs in the presence of elevated partial pressure of hydrogen gas. A biofuel can be refined by catalytic cracking to generate diesel, gasoline, and/or kerosene. Refining can also comprise hydrotreatment.

Biofuels can also be refined by combining them in a unification step, for example by using catalysts, such as platinum or a platinum-rhenium mix. The unification process typically produces hydrogen gas, a by-product which can be used in cracking.

The biofuel can also be refined by altering or rearranging or restructuring hydrocarbons into smaller molecules. There are a number of chemical reactions that occur in the catalytic reforming process of which are known to one of ordinary skill in the arts. Generally, catalytic reforming is performed in the presence of a catalyst and high partial pressure of hydrogen. One common process is alkylation. For example, propylene and butylene are mixed with a catalyst such as hydrofluoric acid or sulfinuric acid.

The biofuel can also be blended or combined into mixtures to obtain an end product. For example, the biofuel can be blended to form gasoline of various grades, gasoline with or without additives, lubricating oils of various weights and grades, kerosene of various grades, jet fuel, diesel fuel, heating oil, and chemicals for making plastics and other polymers. Compositions of the biofuel described herein can be combined or blended with fuel products produced by other means.

In one embodiment, the biofuel produced is a biodiesel. In one embodiment, the biodiesel is fatty acid methyl ester. In one embodiment, the biodiesel has less than 90% hydrogen and carbon atoms by weight. In one embodiment, the fatty acid methyl ester fuel or biodiesel has a hydrogen and carbon content by weight of less than about 89.5%. In one embodiment, the biodiesel is combined or blended with fuel products produced by other means.

Chemical End Products

Other chemicals produced by methods described herein include, but are not limited to, chemicals that can be freed from agricultural product upon incubation with EMO, chemicals produced by EMO by digesting substrate available on stillage, and chemicals produced by EMO as part of its natural life cycle. A chemical can be vitamin, amino acid, carbohydrates or other products of biosynthesis. The stillage resulting from oil processing can include cellulosic materials or materials derived from cellulose such as sucrose, fructose, cellobiose, glucose, xylose, arabinose, mannose, rhamnose, galactose, or fucose. Examples of chemicals produced by an alga is shown in Table 3.

Certain edible algae are known to contain a large amount of vitamin B12. Processes for drying algae and packaging dried algae are known in the art (e.g., U.S. Pat. No. 7,214,378). Alage grown on agricultural product can be provided as multi-vitamin supplement including vitamin B12 in a form of a dried power. In one embodiment, an alga is grown on corn stillage, and subsequently separated from the corn stillage. In another embodiment, an alga is grown on bagasse or extracts obtained from bagasse. In another embodiment, an alga is grown on corn stover or extracts obtained from corn stover. In another embodiment, an alga is grown on fruit peel or extracts obtained from fruit peel. In another embodiment, an alga is grown on seed cake or extracts obtained from seed cake. The separated algae are dried and packaged for human consumption according to methods known in the art.

TABLE 3
Compositions of algal biomass.

| | | Official AOAC |
|---|---|---|
| Parameter | Result | Method |
| Proximate analysis | | |
| Moisture (%) | 5.59 | 930.15 |
| Crude protein (%) | 5.9 | 990.03 |
| Fat (%) | 47.8 | 954.02 |
| Fiber (%) | 2.23 | 978.1 |
| Ash (%) | 6.01 | 942.05 |
| Carbohydrate (%) | 32.47 | By difference |
| Total (%) | 100 | |
| Minerals | | |
| Calcium (%) | 0.035 | 985.01/984.27 |
| Phosphorous (%) | 0.99 | 985.01/984.27 |
| Sodium (%) | 0.3 | 985.01/984.27 |
| Potassium (%) | 1.54 | 985.01/984.27 |
| Sulfur (%) | 0.128 | 923.01 |
| Chloride (%) | 0.02 | 923.01 |
| Magnesium (%) | 0.049 | 985.01/984.27 |
| Manganese (ppm) | 18 | 985.01/984.27 |
| Iron (ppm) | 11 | 985.01/984.27 |
| Zinc (ppm) | 25 | 985.01/984.27 |
| Copper (ppm) | 1 | 985.01/984.27 |
| Cobalt (ppm) | 1.1 | 990.08 |
| Chromium (ppm) | 0.4 | 990.08 |
| Iodine (ppm) | 16.5 | 975.08 |
| Selenium (ppm) | 0.02 | 986.15 |
| Molybdenum (ppm) | 1.3 | 990.08 |
| Lead (ppm) | 0.18 | 990.08 |
| Arsenic (ppm) | 0.06 | 990.08 |
| Mercury (ppm) | <0.05 | 975.08 |
| Cadmium (ppm) | 0.05 | 990.08 |
| Nickel (ppm) | 0.2 | 990.08 |
| Vitamins and | | |
| pigments | | |
| Carotene (mg/lb) | 0 | 970.64 |
| Xanchophyll | 13.5 | 970.64 |
| (mg/lb) | | |
| Riboflavin (mg/lb) | 13.1 | 970.65 |
| True vitamin A | 600 | 974.29 |
| (IU/lb) | | |
| Amino acids (% of | | |
| protein) | | |
| Methionine (%) | 2.24 | 985.28 |
| Cystine (%) | 1.84 | 985.28 |
| Lysine (%) | 5.1 | 994.12 |
| Phenylalanine (%)e | 4.1 | 994.12 |
| Leucine (%) | 8.57 | 994.12 |
| Isoleucine (%) | 3.67 | 994.12 |
| Threonine (%) | 5.51 | 994.12 |
| Valine (%) | 5.92 | 994.12 |
| Histidine (%) | 2.04 | 994.12 |
| Arginine (%) | 6.53 | 994.12 |
| Glycine (%) | 6.53 | 994.12 |
| Aspartic acid (%) | 9.18 | 994.12 |
| Serine (%) | 6.12 | 994.12 |
| Glutamic acid (%) | 12.86 | 994.12 |
| Proline (%) | 4.49 | 994.12 |
| Hydroxyproline (%) | 1.43 | 994.12 |
| Alanine (%) | 10.61 | 994.12 |
| Tyrosine (%) | 2.04 | 994.12 |
| Tryptophan (%) | 1.22 | 988.15 |
| Total AA (%) | 100 | |
| Fatty Acids (% of | | |
| FA) | | |
| Lauric (12:0) | 0 | 963.22 |
| Myristic (14:0) | 0.68 | 963.22 |
| Pentadecanoic (15:0) | 0 | 963.22 |
| Palmitic (16:0) | 8.04 | 963.22 |
| Palmitoleic (16:1) | 0.25 | 963.22 |
| Heptadecanoic (17:0) | 0.09 | 963.22 |
| Heptadecanoic (17:1) | 0 | 963.22 |
| Steric (18:0) | 3.17 | 963.22 |
| Oleic (18:1 u9) | 70.95 | 963.22 |
| Oleic (18:1 u7) | 0 | 963.22 |
| Linoleic (18:2 u6) | 14.99 | 963.22 |
| Linolenic(18:3 u3) | 1.15 | 963.22 |
| Arachidic (20:0) | 0.34 | 963.22 |
| Eicosanoic (20:1 u11) | 0.09 | 963.22 |
| Eicosanoic (20:1 u9) | 0.25 | 963.22 |
| Lignoceric (24:0) | 0 | 963.22 |
| Other | 0 | 963.22 |
| Total FA | 100 | |

Other products that can be produced by an EMO include phytanyl or dibiphytanyl compounds, acetone, 2,3-butanediol, butanol, butyrate, CO₂, formate, glycolate, lactate, malate, propionate, pyruvate, succinate, and other fermentation products.

In addition to chemicals, proteins can be produced by methods described herein. In one embodiment, an alga is grown on an agricultural product to produce an algal protein. In another embodiment, an alga is grown on stillage, seed cake, stover such as corn stover, bagasse or fruit peel for animal consumption. In one embodiment, the animal is a human.

Production of Biofuels and Other Chemicals

In one aspect, agricultural products are converted to fatty acids, biofuel, biodiesel, or other end products by growing an organism, such as a microorganism, producing fatty acids or other chemicals described herein. An organism, such as a microorganism, evolutionarily adapted to grow on agricultural product can metabolize an agricultural product or its extract and produce fatty acids or other end products as part of its natural metabolism. Fatty acids or other end products can be extracted from the organism.

In one embodiment, an agricultural product is hydrolyzed to be used as a culture medium. Hydrolysis can ease the accessibility of carbon or nitrogen sources, such as cellulose, lignin, hemicellulose, or amino acids to microorganisms adapted to grow on these sources. Methods of hydrolysis include, but are not limited to, chemical hydrolysis such as acid or alkaline treatment, heat shock, ultrasonication, enzymatic methods, or combinations thereof. In one embodiment, the mixture is inoculated with at least one Chlorella algae strain that metabolizes said at least one of amino acids, using them as carbon or nitrogen source. In another embodiment, the algal strain produces one or more fatty acids by utilizing one or more amino acids as carbon or nitrogen source. In another embodiment, a separation process is utilized, such as filtering, to recover one or more fatty acids produced form the algae strain. In one embodiment, an algal strain is evoluntionarily modified to tolerate chemical byproducts that are inhibitory of the breaking down process. In another embodiment, agricultural product is seed cake. In one embodiment, the seed cake is Jatropha seed cake. In another embodiment, amino acid from Jatropha seed cake includes cysteine, methionine, valine, isoleucine, leucine, tyrosine, phenylalanine, histidine, lysine, threonine, tryptophan, and also aspartic acid, praline, serine, glycine, alanine, asparagine, glutamine or arginine.

In one embodiment, a composition of the Jatropha seed cake is comprised of remaining fat for about 6.4% of weight of dry matter, ash accounting for about 17.8% of dry weight, a group of protein representing about 26% of dry weight. In another embodiment, the composition further comprises lectin (curcin), phorbol esters, saponins, protease inhibitors and phytates. In another embodiment, the composition further comprises carbohydrates for about 49.7% of weight of the dry weight. Protein compositions of Jatropha curcas seed cake have been described, which is incorporated herein by reference (Makkar et al, Protein concentrate from Jatropha curcas screw-pressed seed cake and toxic and antinutritional factors in protein concentrate, Journal of the Science of Food and Agriculture, volume 88, 1542-1548, 2008).

In one embodiment, a stillage is incubated with an EMO. The stillage can either be pretreated or not treated. A pretreatment can be enzymatic treatment, such as incubation with alpha-amylase. A pretreatment can also be hydration with water or a buffered solution. The stillage can be whole stillage obtained after distillation process or a thin stillage obtained after centrifugation of whole stillage. The stillage can also be WDGS, DDGS, CDS, or DG. Depending on the types of EMO cultivated on stillage, an end product can be a chemical, such as a fatty acid or a protein. In one embodiment, incubating stillage with an organism expressing cellulase can increase the production of sugar molecules. In another embodiment, incubating stillage with an alga can increase the production of algal protein and fatty acids.

Stillage can be hydrolyzed by chemical hydrolysis, alkaline or acidic treatment, heat shock, ultrasonication, or enzymatic method. In one embodiment, hydrolysis occurs prior to pretreatment with an EMO. Inoculation step can be performed in a bioreactor by mixing stillage or any derivative of stillage thereof with a stock of EMO. Inoculation and culture step can be performed in an industrial scale to produce fatty acids, biofuel, biodiesel, or other end product. Alternatively, inoculation and culture step can be performed in a laboratory scale to further evolve the stain to a particular type of stillage.

In one embodiment the stillage is obtained from corn. In another embodiment, the corn stillage is incubated with an alga. In another embodiment, the alga is a heterotrophic alga. In another embodiment, the alga is Chlorella protothecoides.

In one embodiment, bagasse extracts are incubated with an EMO. Preparation of bagasse extracts can include acid or alkali treatment, extraction with organic solvent such as toluene, filtration, heating or other separation methods. Bagasse extracts can be used either exclusively or added to other agricultural product to grow an EMO. In one embodiment, an EMO is adapted specifically to break down lignin from bagasse. In another embodiment, EMO adapted to break down ligin from bagasse is further adapted to break down lignin from corn stover.

In one embodiment, stover extracts are incubated with an EMO. Preparation of stover extracts can include acid or alkali treatment, extraction with organic solvent such as toluene, filtration, heating or other separation methods. Stover extracts can be used either exclusively or added to other agricultural product to grow an EMO. In one embodiment, an EMO is adapted specifically to break down lignin from stover. In another embodiment, EMO adapted to break down ligin from stover is further adapted to break down lignin from bagasse.

In one embodiment, peel extracts are incubated with an EMO. Preparation of peel extracts can include acid or alkali treatment, extraction with organic solvent such as toluene, filtration, heating or other separation methods. Peel extracts can be used either exclusively or added to other agricultural product to grow an EMO.

An end product can be an oil produced by an organism grown on an agricultural product. In one embodiment, the oil is extracted from an organism growing in a culture comprising an agricultural product. In one embodiment, an organism is harvested and dried and then the oil extracted from lysed or destroyed cells. The cells can be chemically lysed or mechanical force can be used to destroy cell walls. Oil can be extracted using an organic solvent such as hexane. Other methods of extracting oil from an organism can also be used as would be obvious to one skilled in the art.

The oil can be used as a biofuel, or combined, refined, or further processed to produce a biofuel. For example, a blend of oil and crude petroleum can be provided in a process as described herein and contacted with a catalytic composition. In another example, a blend of oil and a refined fuel such as gasoline can contacted with a catalytic composition. For example without limitation, the fuel component is selected from the group consisting of the following: fossil fuel, petroleum, a mixture for fuel blending, gasoline, diesel, jet fuel, and any combination thereof.

An oil produced from the organism can be refined, cracked, or both. For example, an oil can be subjected to an RBD (refining bleaching deodorizing) process. In another example, the oil can be fractionated into desired components, such as by distillation. Fractionation can be predetermined by a user or can be set to fractionate the oil into hydrocarbon components of desired sizes, compositions, or shapes.

A Biofuel Plant

In one embodiment a biofuel plant processes an agricultural product, (such as jatropha or castor beans) for use as biofuel. In one embodiment the biofuel is biodiesel, bio-gasoline, bio-oil, biojet fuel or bio-kerosene. In one embodiment this process produces a carbonaceous material by-product (e.g., seedcake) which is cultured with an EMO. In one embodiment an EMO (e.g., a algae or bacteria) is used to produce biodiesel from one or more by-products of biodiesel production. In one embodiment an evolutionarily modified algae has a maximum growth rate at least 25%, preferably 50%, 75%, 100%, 200%, 25%-100%, 25%-100%, 50%-150%, 25-200%, more than 200%, more than 300%, or more than 400% greater than algae of the same species that has not been evolutionarily modified to utilize the one or more by-products of biodiesel production.

In one embodiment a process is disclosed for converting a carbonaceous material into one or more end products (e.g., a lipid or fatty acid) which can be used to produce a fuel. In one embodiment the method comprises culturing one or more EMOs with the carbonaceous material. In one embodiment the one or more EMOs digests or ferments the carbonaceous material into one or more end products (e.g., a lipid, protein, oil, gas or alcohol). In one embodiment the EMO is heterotrophic and evolutionarily modified to process the carbonaceous material as a primary carbon source.; In one embodiment the EMO has a maximum growth rate of at least 25% greater than a non-EMO of the same species (e.g., wildtype) that has not been evolutionarily modified to the carbonaceous material as a primary carbon source. In one embodiment the EMO is an algae or a bacteria. In one embodiment the EMO is algae, such as Chlorophyta (Chlorella or Prototheca), Prasinophyta (Dunaliella), Bacillariophyta (Navicula or Nitzschia), Ochrophyta (Ochromonas), Dinophyta (Gyrodinium) or Euglenozoa (Euglena). Chlorella protothecoides and Dunaliella salina. In another embodiment the EMO algae is Chlorella protothecoides or Dunaliella salina.

In one embodiment, a method for producing a biofuel comprises the steps of cultivating and recovering a microorganism (e.g. algae) that has been evolutionary modified to grow in a culture medium comprising a carbonaceous material; fractionating the microorganism to obtain a first lipid fraction; and transesterifying the first lipid fraction with an alcohol to obtain an alkyl ester fraction and recovering the alkyl esters for use as a biodiesel. Optionally the biodiesel is washed or filtered. In another embodiment a method for producing a biofuel comprises growing an EMO with a by-product of biofuel production; isolating and recovering the EMO from the growing step; fractionating the EMO from the growing step to obtain a first lipid fraction and optionally recovering additional components from the carbohydrates and other chemicals; transesterifying the first fraction such as protein, lipid fraction with an alcohol to obtain alkyl esters as the biofuel product and a second or further by-product of biodiesel production; and recovering the biodiesel product and optionally washing and/or filtering the biodiesel product.

In another embodiment an EMO is cultured with a carbonaceous material to produce an end-product. In one embodiment that end-product is a lipid. In another embodiment the end-product is used as a food, medicine, and nutritional supplement.

In another aspect a carbonaceous material processing and biodiesel manufacturing site is disclosed. In one embodiment, a culture unit (e.g., a reactor or continuous culture machine) is provided for growing an EMO (e.g., algae) that processes a carbonaceous material, wherein the culture unit is adapted for growing the EMO under conditions suitable to produce a desired end-product. In one embodiment these conditions are heterotrophic conditions.

In one embodiment a biofuel manufacturing site comprises one or more of: a reactor unit for chemically treating or transesterifying a first lipid fraction with an alcohol (e.g., methanol) to obtain alkyl esters and optionally a glycerol fraction; a separator unit (e.g., filtration or centrifugation) to separate the alkyl esters from other fractions; a culture unit (e.g., a reactor or continuous culture machine) for growing an EMO (e.g., algae) that processes a carbonaceous material as a primary carbon source, wherein the culture unit is adapted for growing the EMO; a microorganism processing unit (e.g., a fractionating device for obtaining and isolating the naturally occurring lipids of the microorganism) for processing the EMO grown in the culture unit to obtain further lipid fractions; optionally a return unit (e.g., a pump) for sending a lipid fraction to the reactor unit; optionally a by-product recovery unit (e.g., a filtration or centrifugation device) for recovering protein or another by-product from the E M O, after the E M O has been processed in the processing unit; and optionally a bio fuel recovery unit (e.g., a washing device or filtration device) to clean or wash the biofuel.

In one embodiment a biofuel manufacturing site comprises one or more of: a reactor unit for chemically treating or transesterifying a first lipid fraction with an alcohol (e.g., methanol) to obtain alkyl esters and optionally a glycerol fraction; a separator unit (e.g., filtration or centrifugation) to separate the alkyl esters from other fractions; a culture unit (e.g., a reactor or continuous culture machine) for growing an EMO (e.g., algae) that processes a carbonaceous material as a primary carbon source, wherein the culture unit is adapted for growing the EMO; a microorganism processing unit (e.g., a fractionating device for obtaining and isolating the naturally occurring lipids of the microorganism) for processing the EMO grown in the culture unit to obtain further lipid fractions; optionally a return unit (e.g., a pump) for sending a lipid fraction to the reactor unit; optionally a by-product recovery unit (e.g., a filtration or centrifugation device) for recovering protein or another by-product from the E M O, after the E M O has been processed in the processing unit; and optionally a bio fuel recovery unit (e.g., a washing device or filtration device) to clean or wash the biofuel. In another embodiment the biofuel manufacturing site further comprises a plant which extracts a first fatty acid fraction from a biomass, producing a by product that is a carbonaceous material.

In one embodiment a biodiesel manufacturing site comprises one or more of: a reactor unit for chemically treating or transesterifying a first lipid fraction with an alcohol (e.g., methanol) to obtain alkyl esters and optionally a glycerol fraction; a separator unit (e.g., filtration or centrifugation) to separate the alkyl esters from other fractions; a culture unit (e.g., a reactor or continuous culture machine) for growing an EMO (e.g., algae) that processes a carbonaceous material as a primary carbon source, wherein the culture unit is adapted for growing the EMO; a microorganism processing unit (e.g., a fractionating device for obtaining and isolating the naturally occurring lipids of the microorganism) for processing the EMO grown in the culture unit to obtain further lipid fractions; optionally a return unit (e.g., a pump) for sending a lipid fraction to the reactor unit; optionally a by-product recovery unit (e.g., a filtration or centrifugation device) for recovering protein or another by-product from the E M O, after the E M O has been processed in the processing unit; and optionally a biodiesel recovery unit (e.g., a washing device or filtration device) to clean or wash the biodiesel. In another embodiment the biodiesel manufacturing site further comprises a plant which extracts a first fatty acid fraction from a biomass, producing a by product that is a carbonaceous material.

In one embodiment a biofuel manufacturing site processes glycerol to produce a biofuel. In one embodiment the glycerol is unpurified. In another embodiment the glycerol is raw waste from biofuel processing of oil isolated from an agricultural plant (e.g., jatropha or castor beans). In one embodiment glycerol is provided to a culture unit. The culture unit comprises at least one EMO that uses glycerol as a primary carbon source. The EMO is grown and then filtered and/or fractionated to obtain lipids or other end-products. The lipids are provided to a reactor unit to transesterify the fatty acids to produce alkyl esters. The alkyl esters can then be filtered and optionally processed to obtain a biofuel product. In one embodiment another end-product comprises a protein. In one embodiment the proteins can be processed in the culture unit and used as supplemental source of nitrogen and/or carbon by the EMO. In one embodiment any unprocessed glycerol in the culture medium can be returned to the culture unit for further processing. In one embodiment as the reactor unit carries out a transesterification reaction, glycerol is obtained and provided to the culture unit for processing. In another embodiment the biofuel manufacturing site further comprises a plant which extracts a first fatty acid fraction from a biomass, producing glycerol raw waste. In another embodiment the biofuel manufacturing site comprises further a plant which extracts a first fatty acid fraction from a biomass, producing glycerol raw waste and at least one other carbonaceous material.

In one embodiment a biofuel produced by a method or using an EMO disclosed herein is combined with one or more additives. In one embodiment the additive is a petroleum additive, such as inorganic peroxides, organic peroxides, di-t-butylperoxide, alkyl nitrates, ethyl hexyl nitrate, amyl nitrate, nitromethane, ethanol, or vegetable oil.

In one embodiment a biofuel without supplementation is used in a combustion engine. In another embodiment, a biofuel (e.g., biodiesel) is provided in a blend with another petroleum product or petroleum alternative to obtain a fuel (such as gasoline, jet fuel or kerosene), a distillate fuel oil composition; a finished nonfuel product (such as a solvent or lubricating oil); or a feedstock for the petrochemical industry) such as naphtha or a refinery gas). In another embodiment a bio fuel is combined with another petroleum based compounds to produce a solvent, a paint, a lacquer; a printing ink; a lubricating oil; a grease; a wax, a candle, a match, a polish; a jelly; a paving compound (e.g., asphalt); a petroleum based coke; a petroleum based feedstock, a chemical feedstock (such as a feedstock for the manufacture of a chemical, synthetic rubber, or a plastic).

In one embodiment, biofuel produced in accordance with the methods or apparatus disclosed can be used in a diesel engine, or can be blended with petroleum-based distillate fuel oil composition at a ratio such that the resulting petroleum substitute may be in an amount of about 5-95%, 15-85%, 20-80%, 25-75%, 35-50% 50-75%, and 75-95% by weight of the total composition.

In one embodiment the process of fueling a compression ignition internal combustion engine, comprises drawing air into a cylinder of a compression ignition internal combustion engine; compressing the air by a compression stroke of a piston in the cylinder; injecting into the compressed air, toward the end of the compression stroke, a fuel comprising the biodiesel; and igniting the fuel by heat of compression in the cylinder during operation of the compression ignition internal combustion engine. In another embodiment, a biofuel is used as a lubricant.

In another embodiment, a biofuel is processed to obtain a hydrocarbon such as a paraffin (e.g., methane, ethane, propane, butane, isobutane, pentane, or hexane), an aromatic (e.g., benzene or naphthalene), a cycloalkane (e.g., cyclohexane or methyl cyclopentane), an alkene (e.g., ethylene, butene, or isobutene), or an alkyne (e.g., acetylene, or butadienes).

Converting Agricultural Products to Ethanol

In one embodiment, an organism, method or system described herein converts a biomass to ethanol. In one embodiment, an organism, method or system described herein converts an agricultural product to ethanol. In one embodiment, the method can include preparing the plant material for saccharification, converting the prepared plant material to sugars without cooking, and fermenting the sugars.

In one embodiment, plant material can be prepared for saccharification by any a variety of methods, e.g., by grinding, to make the agricultural products available for saccharification and fermentation. In one embodiment, the vegetable material can be ground so that a substantial portion, e.g., a majority, of the ground material fits through a sieve. In one embodiment, the reduced plant material can be mixed with liquid. Other methods of plant material reduction are available. For example, vegetable material, such as kernels of corn, can be ground with a ball mill, a roller mill, a hammer mill, or another mill known for grinding vegetable material, and/or other materials for the purposes of particle size reduction. The use of emulsion technology, rotary pulsation, and other means of particle size reduction can be employed to increase surface area of plant material while raising the effectiveness of flowing the liquefied media.

Conversion can include converting reduced plant material to sugars that can be fermented by a microorganism. This conversion can be effected by saccharifying the reduced plant material with an enzyme preparation, such as a saccharifying enzyme composition. A saccharifying enzyme composition can include any of a variety of known enzymes suitable for converting reduced plant material to fermentable sugars, such as amylases (e.g., α-amylase and/or glucoamylase).

In one embodiment, conversion is fermenting sugars from reduced plant material to ethanol. Fermenting can be effected by a microorganism, such as yeast. Fermentation can take place in varying pH or in constant pH. Fermentation can take place in varying temperature.

A conversion process can be simultaneous, converting reduced plant material to sugars and fermenting those sugars with a microorganism.

Ethanol can be recovered from the fermentation mixture by any of a variety of known processes, such as by distilling. The remaining stillage includes both liquid and solid material. The liquid and solid can be separated by, for example, centrifugation.

Plant Material Reduction

Preparing the plant material can employ any of a variety of techniques for plant material reduction. For example, a method of preparing plant material can employ emulsion technology, rotary pulsation, sonication, magnetostriction, or ferromagnetic materials. These methods of plant material reduction can be employed for substrate pretreatment. These methods can include electrical to mechanical, mechanical to electrical, pulse, and sound based vibrations at varying speeds. This can provide varying frequencies over a wide range of frequencies, which can be effective for pretreating the plant material and/or reducing particle size.

In one embodiment, a method is related to vibrating plant material and cavitating the fluid containing the plant material. This can result in disrupting the plant material and/or decreasing the size of the plant material. In certain embodiments, a method is related to treating plant material with emulsion technology, with rotary pulsation, with magnetostriction, or with ferromagnetic materials. This can result in disrupting the plant material and/or decreasing the size of the plant material. In one embodiment, a method includes sonicating the plant material. This can result in disrupting the plant material and/or decreasing the size of the plant material.

In one embodiment, a method can include employing rotary pulsation for reducing plant material. The method can include rotary pulsating the plant material at a frequency (e.g., measured in Hz), power (e.g., measured in watts), and for a time effective to reduce (or to assist in reducing) the particle size to sizes described hereinabove. Such rotary pulsating can be carried out with known apparatus, such as apparatus described in U.S. Pat. No. 6,648,500, the disclosure of which is incorporated herein by reference.

In one embodiment, a method can include employing pulse wave technology for reducing plant material. The method can include rotary pulsing the plant material at a frequency, power, and for a time effective to reduce (or to assist in reducing) the particle size to sizes described hereinabove. Such pulsing can be carried out with known apparatus, such as apparatus described in U.S. Pat. No. 6,726,133, the disclosure of which is incorporated herein by reference.

Fractionation

In one embodiment, plant material can be fractionated into one or more components. For example, a vegetable material such as a cereal grain or corn can be fractionated into components such as fiber (e.g., corn fiber), germ (e.g., corn germ), and a mixture of starch and protein (e.g., a mixture of corn starch and corn protein). In one embodiment any of these fractionated components can be used by an EMO to produce a desired end-product (such as fatty acids, a biofuel) Fractionation of corn or another plant material can be accomplished by any of a variety of methods or apparatus.

In one embodiment, germ and fiber components of a vegetable material can be fractionated and separated from the remaining portion of the vegetable material. In one embodiment, the remaining portion of the vegetable material (e.g., corn endosperm) can be further milled and reduced in particle size and then combined with the larger pieces of the fractioned germ and fiber components for fermenting.

Fractionation can be accomplished by any of a variety of methods and apparatus, such as those disclosed in U.S. Patent Application Publication No. 2004/0043117, the disclosure of which is incorporated herein by reference. Suitable methods and apparatus for fractionation include a sieve, sieving, and elutriation. Suitable apparatus include a frictional mill such as a rice or grain polishing mill (e.g., those manufactured by Satake, Kett, or Rapsco)

Saccharification

Methods and compositions described herein can include converting reduced plant material to sugars that can be fermented by a microorganism. This conversion can be effected by saccharifying the reduced plant material with any of a variety of known saccharifying enzyme compositions. In one embodiment, the saccharifying enzyme composition includes an amylase, such as an alpha amylase (e.g., an acid fungal amylase). The enzyme preparation can also include glucoamylase. In one embodiment, a method employs acid fungal amylase for hydrolyzing raw starch.

Saccharifying can be conducted without cooking. For example, saccharifying can be conducted by mixing source of saccharifying enzyme composition (e.g., commercial enzyme), yeast, and fermentation ingredients with ground grain and process waters without cooking.

In one embodiment, saccharifying can include mixing the reduced plant material with a liquid, which can form a slurry or suspension and adding saccharifying enzyme composition to the liquid. In one embodiment, the method includes mixing the reduced plant material and liquid and then adding the saccharifying enzyme composition. Alternatively, adding enzyme composition can precede or occur simultaneously with mixing.

Suitable liquids include water and a mixture of water and process waters, such as stillage (backset), scrubber water, evaporator condensate or distillate, side stripper water from distillation, or other ethanol plant process waters.

Saccharification can employ any of a variety of known enzyme sources (e.g., a microorganism) or compositions to produce fermentable sugars from the reduced plant material. In one embodiment, the saccharifying enzyme composition includes an amylase, such as an alpha amylase (e.g., an acid fungal amylase) or a glucoamylase.

In one embodiment, saccharification is conducted at a pH of about 6.0 or less, pH of about 3.0 to about 6.0, about 3.5 to about 6.0, about 4.0 to about 5.0, about 4.0 to about 4.5, about 4.5 to about 5.0, or about 4.5 to about 4.8. In one embodiment, saccharification is conducted at a pH of about 4.1 to about 4.6 or about 4.9 to about 5.3. The initial pH of the saccharification mixture can be adjusted by addition of, for example, ammonia, sulfuric acid, phosphoric acid, process waters (e.g., stillage (backset), evaporator condensate (distillate), side stripper bottoms, and the like), and the like. Activity of certain saccharifying enzyme compositions (e.g., one including acid fungal amylase) can be enhanced at pH lower than the above ranges.

In one embodiment, saccharification is conducted at a temperature of about 25 to about 40° C. or about 30 to about 35° C.

In one embodiment, saccharifying can be carried out employing quantities of saccharifying enzyme composition selected to maintain low concentrations of dextrin in the fermentation broth.

Glucoamylase

In certain embodiments, a method can employ a glucoamylase. Glucoamylase is also known as amyloglucosidase and has the systematic name 1,4-alpha-D-glucan glucohydrolase (E.C. 3.2.1.3). Glucoamylase refers to an enzyme that removes successive glucose units from the non-reducing ends of starch. For example, certain glucoamylases can hydrolyze both the linear and branched glucosidic linkages of starch, amylose, and amylopectin. A variety of suitable glucoamylases are known and commercially available. For example, suppliers such as Novozymes and Genencor provide glucoamylases. The glucoamylase can be of fungal origin.

In one embodiment, the amount of glucoamylase employed in a process can vary according to the enzymatic activity of the amylase preparation. Suitable amounts include about 0.05 to about 6.0 glucoamylase units (AGU) per gram dry solids reduced plant material (e.g., DSC). In one embodiment, the reaction mixture can include about 1 to about 6 AGU per gram dry solids reduced plant material (e.g., DSC). In one embodiment, the reaction mixture can include about 1 to about 3 AGU per gram dry solids reduced plant material (e.g., DSC). In one embodiment, the reaction mixture can include about 1 to about 2.5 (e.g., 2.4) AGU per gram dry solids reduced plant material (e.g., DSC). In one embodiment, the reaction mixture can include about 1 to about 2 AGU per gram dry solids reduced plant material (e.g., DSC). In one embodiment, the reaction mixture can include about 1 to about 1.5 AGU per gram dry solids reduced plant material (e.g., DSC). In one embodiment, the reaction mixture can include about 1.2 to about 1.5 AGU per gram dry solids reduced plant material (e.g., DSC).

Acid Fungal Amylase

In certain embodiments, a method employs an α-amylase. The α-amylase can be one produced by fungi. The α-amylase can be one characterized by its ability to hydrolyze carbohydrates under acidic conditions. An amylase produced by fungi and able to hydrolyze carbohydrates under acidic conditions is referred to herein as acid fungal amylase, and is also known as an acid stable fungal α-amylase. Acid fungal amylase can catalyze the hydrolysis of partially hydrolyzed starch and large oligosaccharides to sugars such as glucose. The acid fungal amylase that can be employed in a process can be characterized by its ability to aid the hydrolysis of raw or native starch, enhancing the saccharification provided by glucoamylase. In one embodiment, the acid fungal amylase produces more maltose than conventional (e.g., bacterial) α-amylases.

Suitable acid fungal amylase can be isolated from any of a variety of fungal species, including Aspergillus, Rhizopus, Mucor, Candida, Coriolus, Endothia, Enthomophtora, Irpex, Penicillium, Sclerotium and Torulopsis species. In one embodiment, the acid fungal amylase is thermally stable and is isolated from an Aspergillus species, such as A. niger, A. saitoi or A. oryzae, from a Mucor species such as M. pusillus or M miehei, or from Endothia species such as E. parasitica. In one embodiment, the acid fungal amylase is isolated from Aspergillus niger. The acid fungal amylase activity can be supplied as an activity in a glucoamylase preparation, or it can be added as a separate enzyme. A suitable acid fungal amylase can be obtained from Novozymes, for example in combination with glucoamylase.

The amount of acid fungal amylase employed in a process can vary according to the enzymatic activity of the amylase preparation. Suitable amounts include about 0.1 to about 10 acid fungal amylase units (AFAU) per gram of dry solids reduced plant material (e.g., dry solids corn (DSC)). In one embodiment, the reaction mixture can include about 0.05 to about 3 AFAU/gram dry solids reduced plant material (e.g., DSC). In one embodiment, the reaction mixture can include, about 0.1 to about 3 AFAU/gram dry solids reduced plant material (e.g., DSC). In one embodiment, the reaction mixture can include about 0.3 to about 3 AFAU/gram dry solids reduced plant material (e.g., DSC). In one embodiment, the reaction mixture can include about 1 to about 2 AFAU/gram dry solids reduced plant material (e.g., DSC).

Fermenting

Any of a variety of yeasts can be employed as the yeast starter in a process. Suitable yeasts include any of a variety of commercially available yeasts, such as commercial strains of Saccharomyces cerevisiae. Suitable strains include “Fali” (Fleischmann's), Thermosac (Alltech), Ethanol Red (LeSafre), and BioFerm AFT (North American Bioproducts). In one embodiment, the yeast is selected to provide rapid growth and/or high fermentation rates in the presence of high temperature and high ethanol levels.

In one embodiment, the yeast employed is an evolutionarily modified yeast having enhanced stress resistance.

Yeast can be added to the fermentation by any of a variety of methods known for adding yeast to fermentation processes. For example, yeast starter can be added as a dry batch, or by conditioning/propagating. In one embodiment, yeast starter is added as a single inoculation. In one embodiment, yeast is added to the fermentation during the fermenter fill at a rate of 5 to 100 pounds of active dry yeast (ADY) per 100,000 gallons of fermentation mash. In one embodiment, the yeast can be acclimated or conditioned by incubating about 5 to 50 pounds of ADY per 10,000 gallon volume of fermenter volume in a prefermenter or propagation tank. Incubation can be from 8 to 16 hours during the propagation stage, which is also aerated to encourage yeast growth. The prefermenter used to inoculate the main fermenter can be from 1 to 10% by volume capacity of the main fermenter, for example, from 2.5 to 5% by volume capacity relative to the main fermenter.

In one embodiment, fermentation is conducted at a pH of about 6 or less, pH of about 3 to about 6, about 3 to about 4.5, about 3.5 to about 6, about 4 to about 5, about 4 to about 4.5, about 4.5 to about 5, or about 4.5 to about 4.8. The initial pH of the fermentation mixture can be adjusted by addition of, for example, ammonia, sulfuric acid, phosphoric acid, process waters (e.g., stillage (backset), evaporator condensate (distillate), side stripper bottoms, and the like), and the like.

In one embodiment, fermentation is conducted for about to 25 (e.g., 24) to about to 150 hours, about 25 (e.g., 24) to about 96 hours, about 40 to about 96 hours, about 45 (e.g., 44) to about 96 hours, about 48 (e.g., 47) to about 96 hours. For example, fermentation can be conducted for about 30, about 40, about 50, about 60, or about 70 hours. For example, fermentation can be conducted for about 35, about 45, about 55, about 65, or about 75 hours.

In one embodiment, fermentation is conducted at a temperature of about 25 to about 40° C. or about 30 to about 35° C. In one embodiment, during fermentation the temperature is decreased from about 40° C. to about 30° C. or about 25° C., or from about 35° C. to about 30° C., during the first half of the fermentation, and the temperature is held at the lower temperature for the second half of the fermentation. In one embodiment, the temperature can be decreased as ethanol is produced. For example, in one embodiment, during fermentation the temperature can be as high as about 99° F. and then reduced to about 79° F. This temperature reduction can be coordinated with increased ethanol titers (%) in the fermenter.

In one embodiment, a method includes solids staging. Solids staging includes filling at a disproportionately higher level of solids during the initial phase of the fermenter fill cycle to increase initial fermentation rates. The solids concentration of the mash entering the fermenter can then be decreased as ethanol titers increase and/or as the fermenter fill cycle nears completion. In one embodiment, the solids concentration can be about 40% (e.g. 41%) during the first half of the fermentation fill. This can be decreased to about 25% after the fermenter is 50% full and continuing until the fermenter fill cycle is concluded. In the above example, such a strategy results in a full fermenter with solids at 33%.

Simultaneous Saccharification and Fermentation

In one embodiment, saccharification can be performed by simultaneously converting reduced plant material to sugars and fermenting those sugars with a microorganism. Simultaneous saccharification and fermenting can be conducted using the reagents and conditions described above for saccharification and fermenting.

In one embodiment, saccharification and fermentation are conducted at a temperature of about 25 to about 40° C. or about 30 to about 35° C. In one embodiment, during saccharification and fermentation the temperature is decreased from about 40 to about 25° C. or from about 35 to about 30° C. during the first half of the saccharification, and the temperature is held at the lower temperature for the second half of the saccharification.

In another embodiment, saccharification and fermentation are conducted at a pH of about 6 or less, pH of about 3 to about 6, about 3.5 to about 6, about 4 to about 5, about 4 to about 4.5, about 4.5 to about 5, or about 4.5 to about 4.8. The initial pH of the saccharification and fermentation mixture can be adjusted by addition of, for example, ammonia, sulfuric acid, phosphoric acid, process waters (e.g., stillage (backset), evaporator condensate (distillate), side stripper bottoms, and the like), and the like.

In another embodiment, saccharification and fermentation are conducted for about to 25 (e.g., 24) to about to 150 hours, about 25 (e.g., 24) to about 72 hours, about 45 to about 55 hours, about 50 (e.g., 48) to about 96 hours, about 50 to about 75 hours, or about 60 to about 70 hours. For example, saccharification and fermentation can be conducted for about 30, about 40, about 50, about 60, or about 70 hours. For example, saccharification and fermentation can be conducted for about 35, about 45, about 55, about 65, or about 75 hours.

In another embodiment, Simultaneous saccharification and fermenting can be carried out employing quantities of enzyme and yeast selected to maintain high concentrations of yeast and high levels of budding of the yeast in the fermentation broth. For example, a process can employ quantities of enzyme and yeast selected to maintain yeast at or above about 200 cells/mL, at or above about 300 cells/mL, or at about 300 to about 600 cells/mL.

In another embodiment, Simultaneous saccharification and fermenting can be carried out employing quantities of enzyme and yeast selected for effective fermentation without added exogenous nitrogen; without added protease; and/or without added backset. Backset can be added, if desired, to consume process water and reduce the amount of wastewater produced by the process. In addition, a process maintains low viscosity during saccharification and fermenting.

In another embodiment, Simultaneous saccharification and fermenting can be carried out employing quantities of enzyme and yeast selected to maintain low concentrations of soluble sugar in the fermentation broth. In one embodiment, Simultaneous saccharification and fermenting can be carried out employing quantities of enzyme and yeast selected to maintain low concentrations of glucose in the fermentation broth. For example, a process can employ quantities of enzyme and yeast selected to maintain glucose at levels at or below about 2 wt-% during saccharification and fermenting. In one embodiment, Simultaneous saccharification and fermenting can be carried out employing quantities of enzyme and yeast selected to maintain low concentrations of maltose (DP2) in the fermentation broth.

In another embodiment, Simultaneous saccharification and fermenting can be carried out employing quantities of enzyme and yeast selected to maintain low concentrations of dextrin in the fermentation broth. For example, a process can employ quantities of enzyme and yeast selected to maintain maltotriose (DP3) at levels at or below about 0.5 wt-%, at or below about 0.2 wt-%, or at or below about 0.1 wt-%.

In another embodiment, Simultaneous saccharification and fermenting can be carried out employing quantities of enzyme and yeast selected to maintain low concentrations of fusel oils in the fermentation broth. For example, a process can employ quantities of enzyme and yeast selected to maintain fusel oils at levels at or below about 0.4 to about 0.5 wt-%.

For example, Simultaneous saccharification and fermenting can employ acid fungal amylase at about 0.05 to about 10 AFAU per gram of dry solids reduced plant material (e.g., DSC) and glucoamylase at about 0.5 to about 6 AGU per gram dry solids reduced plant material (e.g., DSC). For example, Simultaneous saccharification and fermenting can employ acid fungal amylase at about 0.1 to about 10 AFAU per gram of dry solids reduced plant material (e.g., DSC) and glucoamylase at about 0.5 to about 6 AGU per gram dry solids reduced plant material (e.g., DSC). For example, Simultaneous saccharification and fermenting can employ acid fungal amylase at about 0.3 to about 3 AFAU per gram of dry solids reduced plant material (e.g., DSC) and glucoamylase at about 1 to about 3 AGU per gram dry solids reduced plant material (e.g., DSC). For example, Simultaneous saccharification and fermenting can employ acid fungal amylase at about 1 to about 2 AFAU per gram of dry solids reduced plant material (e.g., DSC) and glucoamylase at about 1 to about 1.5 AGU per gram dry solids reduced plant material (e.g., DSC).

Additional Ingredients for Saccharification and/or Fermentation

In one embodiment, the saccharification and/or fermentation mixture can include additional ingredients to increase the effectiveness of the process. For example, the mixture can include added nutrients (e.g., yeast micronutrients), antibiotics, salts, added enzymes, and the like. Nutrients can be derived from stillage or backset added to the liquid. Suitable salts can include zinc or magnesium salts, such as zinc sulfate, magnesium sulfate, and the like. Suitable added enzymes include those added to conventional processes, such as protease, phytase, cellulase, hemicellulase, exo- and endo-glucanase, xylanase, and the like.

Recovering Ethanol

In one embodiment, stillage includes both liquid and solid material. The liquid and solid can be separated by, for example, centrifugation. The recovered liquid, thin stillage, can be employed as at least part of the liquid for forming the saccharification and fermentation mixture for subsequent batches or runs.

The recovered solids, distiller's dried grain, include unfermented grain solids and spent yeast solids. Thin stillage can be concentrated to a syrup, which can be added to the distiller's dried grain and the mixture then dried to form distiller's dried grain plus solubles. Distiller's dried grain and/or distiller's dried grain plus solubles can be sold as animal feed.

Fractionation of Solids from Fermentation

Large pieces of germ and fiber can ferment with the residual starch in the fermenter. After fermentation, the fractions could be removed prior to or after distillation. Removal can be effected with a surface skimmer before to distillation. In one embodiment, screening can be performed on the beer. The screened material can then be separated from the ethanol/water mix by, for example, centrifugation and rotary steam drum drying, which can remove the residual ethanol from the cake.

In one embodiment, all the components are blended and dried together. The fiber and germ can be removed from the finished product by aspiration and/or size classification. The fiber from the DDGS can be aspirated. Removal of fiber by aspiration after drying can increase the amount of oil and protein in the residual DDGS.

In one embodiment, fractionation can employ the larger fiber and germ pieces to increase the particle size of that part of the DDGS derived from the endosperm, as well as to improve syrup carrying capacity. A ring dryer disintegrator can provide some particle size reduction and homogenization.

Drying Wet Cake to Make Distiller's Dried Grains

In one embodiment, fermented product includes ethanol, other liquids, and solid material. Centrifugation and/or distillation of the products can yield solids known as wet cake and liquids known as thin stillage. The wet cake can be dried to produce distiller's dried grain. The thin stillage can be concentrated to a syrup, which can be added to the wet cake or distiller's dried grain and the mixture then dried to form distiller's dried grain plus solubles. A method can include drying the wet cake to produce distiller's dried grain. A method can include drying the syrup plus distiller's dried grain to produce distiller's dried grain plus solubles. The distiller's dried grain can be produced from whole grain (e.g., corn) or from fractionated grain (e.g., corn). The present method can produce high protein distiller's dried grain and/or distiller's dried grain with improved physical characteristics. Such distiller's dried grains are described hereinbelow.

Conventional ethanol production processes employed drum dryers. Advantageously, in one embodiment, the present method and system can employ a flash or ring dryer. Flash or ring dryers have not previously been employed in processes like the present one. Configurations of flash and ring dryers are known. Briefly, a flash or ring dryer can include a vertical column through which a pre-heated air stream moves the wet cake. For example, a flash or ring dryer can include one or more inlets that provide entry of heat or heated air into the dryer. This dries the wet cake. The dried wet cake is transported to the top of a column. In a ring dryer, further drying can be accomplished by moving the wet cake through one or more rings connected to the column. For example, a ring dryer can include one or more inlets through which heated air enters a ring structure which propels or circulates the wet cake in or around the ring structure. The dried wet cake can then be pneumatically conveyed to down-stream separating equipment such as a cyclone or dust collector.

The present method can include employing a flash dryer to dry (i.e., flash drying) the wet cake and to produce distiller's dried grain. The present method can include employing a flash dryer to dry (i.e., flash drying) the syrup plus distiller's dried grain to produce distiller's dried grain plus solubles. Employing a flash dryer can produce high protein distiller's dried grain and/or distiller's dried grain with improved physical characteristics. Such distiller's dried grains are described hereinbelow.

The present method can include employing a ring dryer to dry (i.e., ring drying) the wet cake and to produce distiller's dried grain. The present method can include employing a ring dryer (i.e., ring drying) to dry the syrup plus distiller's dried grain to produce distiller's dried grain plus solubles. Employing a ring dryer can produce high protein distiller's dried grain and/or distiller's dried grain with improved physical characteristics. Such distiller's dried grains are described hereinbelow.

The present method can include employing a fluid bed dryer to dry (i.e., fluid bed drying) the wet cake and to produce distiller's dried grain. The present method can include employing a fluid bed dryer to dry (i.e., fluid bed drying) the syrup plus distiller's dried grain to produce distiller's dried grain plus solubles. Employing a fluid bed dryer can produce high protein distiller's dried grain and/or distiller's dried grain with improved physical characteristics. Such distiller's dried grains are described hereinbelow.

The present method can include adding syrup (backset or thin stillage) to the wet cake before, during, or after drying. In one embodiment, the present method includes adding syrup (backset or thin stillage) to the wet cake during drying. For example, the method can include mixing wet cake and syrup in the dryer. For example, the method can include flowing or injecting syrup into the flash, ring, or fluid bed dryer. In one embodiment, the present method includes adding syrup into the column or ring of the dryer in the presence of wet cake and/or distiller's dried grain.

In one ebodiment, flash and/or ring dryers differ from rotary or drum dryers by providing decreased exposure of wet cake to high temperatures of the drying process. A rotary or drum dryer generally has high temperature metal that is in prolonged contact with the wet cake product. It is believed that prolonged contact of this high temperature metal with the wet cake can result in browned, burned, or denatured distiller's dried grains or distiller's dried grains plus solubles. Further, the internal air temperature can be higher in a rotary or drum dryer.

Accordingly, in one embodiment, the present method can include drying the wet cake or wet cake plus syrup for a shorter time than employed with a rotary or drum dryer, and obtaining distiller's dried grain or distiller's dried grain plus solubles that has been sufficiently dried. Accordingly, in one embodiment, the present method can include drying the wet cake or wet cake plus syrup at a lower temperature than employed with a rotary or drum dryer, and obtaining distiller's dried grain or distiller's dried grain plus solubles that has been sufficiently dried. In one embodiment, the method includes changing the drying temperature during drying.

In certain embodiments, such drying systems and methods can provide one or more advantages such as decreased energy consumption in drying, decreased leakage from the drying system.

In one embodiment the flash or ring dryer(s) is used to change the conditions inside the dryer system to increase or decrease temperature. In another embodiment the flash or ring dryer(s) is used to change the conditions inside the dryer system to increase or decrease the moisture. In another embodiment the flash or ring dryer(s) is used to change the conditions inside the dryer system to increase or decrease recycle speed. In another embodiment the flash or ring dryer(s) is used to change the conditions inside the dryer system to increase or decrease the feed rate into the dryer system.

Continuous Fermentation

A process can be run via a batch or continuous process. A continuous process includes moving (pumping) the saccharification and/or fermenting mixtures through a series of vessels (e.g., tanks) to provide a sufficient duration for the process. For example, a multiple stage fermentation system can be employed for a continuous process with 48-96 hours residence time. For example, reduced plant material can be fed into the top of a first vessel for saccharification and fermenting. Partially incubated and fermented mixture can then be drawn out of the bottom of the first vessel and fed in to the top of a second vessel, and so on.

In one embodiment the present method is more suitable than conventional methods for running as a continuous process. In aonther embodiment a process provides reduced opportunity for growth of contaminating organisms in a continuous process. At present, the majority of dry grind ethanol facilities employ batch fermentation technology. This is in part due to the difficulty of preventing losses due to contamination in these conventional processes. For efficient continuous fermentation using traditional liquefaction technology, a separate saccharification stage prior to fermentation is used to pre-saccharify the mash for fermentation. Such pre-saccharification insures that there is adequate fermentable glucose for the continuous fermentation process.

The present method achieves efficient production of high concentrations of ethanol without a liquefaction or saccharification stage prior to fermentation. The present method can provide low concentrations of glucose and efficient fermentation. In the present method, it appears that the glucose is consumed rapidly by the fermenting yeast cell. It is believed that such low glucose levels reduce stress on the yeast, such as stress caused by osmotic inhibition and bacterial contamination pressures. In one embodiment ethanol levels greater than 18% by volume can be achieved in about 45 to about 96 hours.

In one embodiment, a process can ferment a portion of a reduced plant material, such as corn. For example, the process can ferment at least one of endosperm, fiber, or germ. A process can increase ethanol production from such a portion of corn. In one embodiment, a process can saccharify and ferment endosperm. Endosperm fermentation is lower in free amino nitrogen (FAN) towards the beginning of fermentation due to the removal of germ, which contains FAN. A process can, for example, preserve the FAN quality of the endosperm compared to conventional high temperature liquefaction. In one embodiment endosperm FAN is used to increase flexibility and efficiency of fermentation.

In one embodiment, a process can employ endogenous enzyme activity in the grain. In one embodiment, dramatic increase in FAN in whole corn and defibered corn fermentations are reached compared to the initial mash slurry.

Conventional grain dry milling operations separate germ (containing oil) and bran or pericarp (fiber fraction) from the endosperm (starch and protein) portion of the grain using a series of steps and procedures. These steps and procedures include: grain cleaning, tempering, degerming, particle size reduction, roller milling, aspirating, and sifting. This process differs from the traditional wet milling of grains (commonly corn) which are more expensive and water intensive, but capable of achieving cleaner separations of the components of the grain. Dry milling processes offer a version of separating components using lower capital costs for facilities. In one embodiment less water is used for operation. In anoother embodiment the tempering process in dry milling uses less water than wet milling.

In one embodiment the competitiveness of dry grain fractionation processes is enhanced when a process is used for ethanol conversion of these fractions. Traditionally dry milling processes produce various grades of each fraction (germ, bran, and endosperm). In one embodiment, the present method provides bran and endosperm fractions that can be more readily fermented. Depending on the desired purity of each fraction, the fractions can either be pooled to create composites of each stream, or the fractions can be processed individually.

### EXAMPLES

### Example 1

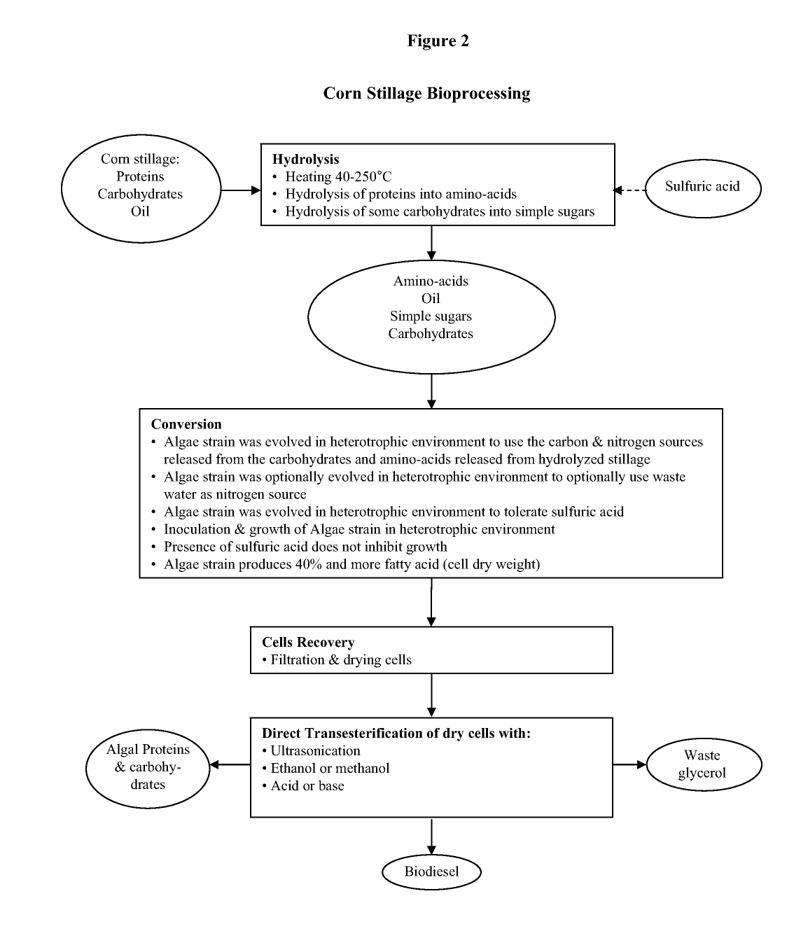

Corn stillage is hydrolyzed with or without sulfur treatment. Carbon or nitrogen sources released from the stillage are fed into an organism suitable for producing hydrocarbon molecules. Alternatively, an alga is evolved to grow directly on stillage is used to convert carbon or nitrogen sources to hydrocarbon molecules. Cells grown on stillage are separated from the stillage and subjected to further purification steps. Transesterification and further processing, such as cracking, if necessary, are employed to produce biofuel. Hydrocarbons extracted from cells are used directly as biodiesel (FIG. 2).

### Example 2

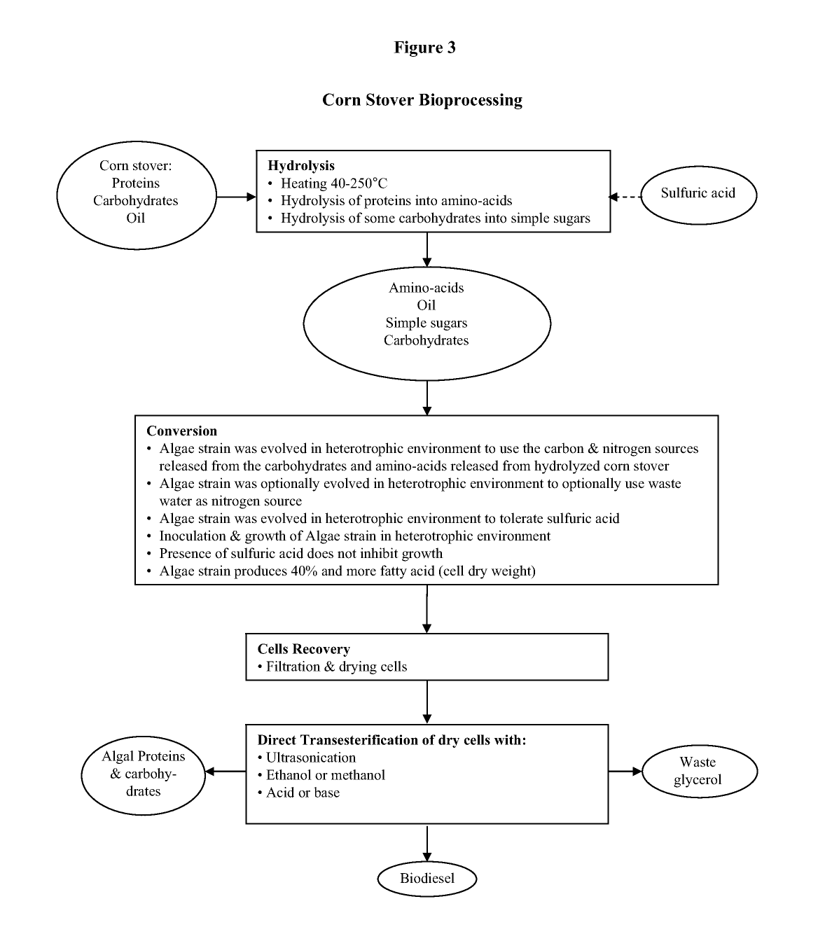

Corn stover is hydrolyzed with or without sulfur treatment. Carbon or nitrogen sources released from the stover are fed into an organism suitable for producing hydrocarbon molecules. Alternatively, an alga is evolved to grow directly on stover is used to convert carbon or nitrogen sources to hydrocarbon molecules. Cells grown on stover are separated from the stover and subjected to further purification steps. Transesterification and further processing, such as cracking, if necessary, are employed to produce biofuel. Hydrocarbons extracted from cells are used directly as biodiesel (FIG. 3).

### Example 3

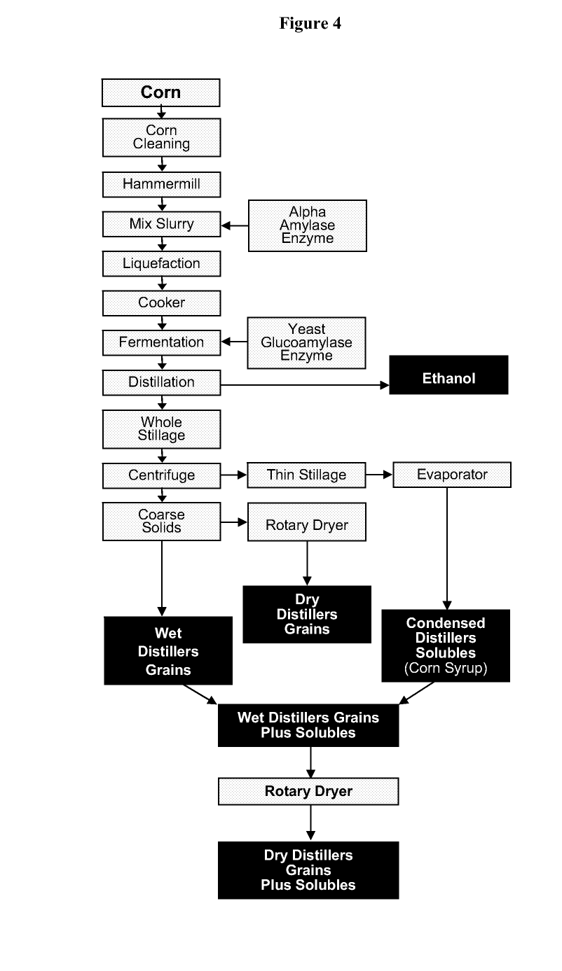

At various points of corn processing toward ethanol production, stillages are produced by various physical methods such as drying, centrifugation, or evaporation (FIG. 4). At various points in the illustrated process, stillage can be withdrawn from the process and used for the production of biofuel, biodiesel, and other chemicals. Stillages that can be withdrawn from the process include whole stillage produced after the distillation step, thin stillage produced after the centrifugation step, WDG from coarse solids, DDG produced after drying in rotatory dryer, condensed distillers solubes (CDS) produced after evaporation step, WDGS from WDG and CDS, DDGS from WDGS. Stillages obtained by these processes are further utilized for production of fatty acids, biodiesel, or other end product by methods described herein, such as Examples 1 and 2.

### Example 4

The present example relates to a possible preparation of the sugarcane bagasse extract prior to evolve micro-organism to better metabolizing its components. Sugarcane bagasse extract is prepared by the following steps. First, sugarcane bagasse is dried at 65° C. Second, the heated sugarcane bagasse is air-dried. After air-dry, the sugarcane bagasse is then milled through a 2 mm diameter mesh. A fine grind of sugarcane bagasse is mixed with distilled water. About 15 g of sample bagasse is placed in 45 ml of water and sterilized by autoclaving at 121° C. for 20 minutes.

### Example 5

A strain of Pleurotus ostreatus, Lentinula edodes, P. eryngii, P. salmoneostramineus, or C. subvermispora is obtained from a vendor and placed in a continuous culture device. Initially, the strain is cultured on yeast extract. The culture is grown at 26° C. After reaching an exponential growth phase, the initial culture is divided into smaller volumes of culture. Each subculture is put to artificial evolution process by inoculating the subculture with a medium prepared for artificial evolution. A medium for artificial evolution is prepared by mixing yeast extract with sugarcane bagasse extract. Initially, an inoculum is grown in a medium containing 99% yeast extract with 1% sugarcane bagasse extract. In subsequent culture, each culture from previous step is transfered to a medium having higher percentage of sugarcane bagasse extract. The process is continuously repeated until a strain that grows on 99% or higher percentage of sugarcane bagasse extract emerges.

### Example 6

Apple peels will be prepared to evolutionarily modify a micro-organism to better metabolize the apple peel's components. A strain of Candida utilis, or Pleurotus ostreatus is obtained from a vendor and placed in a continuous culture device. Initially, the strain is cultured in medium containing yeast extract, malt extract, peptone, and glucose. The strain is grown to reach an exponential phase. Afterward, the culture is divided into smaller volume and each subculture is subjected to artificial evolution process to adapt to growth on utilizing medium comprised of 99% or higher percentage of apple peel extract. Apple peel extract is prepared as the following: wet apple peel is mixed with water in 1 to 2 ratio (weight/volume). The mixture is sterilized by autoclaving at 121° C. for 20 minutes. The sterilized liquid is cooled and mixed with NaCl solution followed by centrifugation at 850×g for 10 minutes.

### Example 7

Ripe apple is collected and the outer epidermis zone is peeled from the apple. The peel is immersed in ethanol (1:4 w/v) and grinded in a blender for 20 seconds followed by boiling for 10 minutes. The mixture is filtered through a sintered-glass filter. An alcohol-insoluble solid is prepared by washing with 70% ethanol until the filtrate becomes colorless. The alcohol-insoluble solid is dried by solvent exchange (96%, ethanol, acetone) and is air-dried at 35° C. The alcohol-insoluble solid is further treated either chemically or enzymatically (e.g., endoglucanase or pectin methyltransferase) to break down the solid into pectin or lower alkyl groups. The broken-down material is used for growing and adapting microorganisms to robustly grow on pectin-derived carbohydrates.

Mandarin citrus peel with dry matter content having at least 20% of total weight are collected and cut into small pieces. The citrus peel is further broken down to less than 7 mm size particles in a cutter machine. Particles are introduced into a pressure reactor. Thermohydrolysis is performed in the pressure reactor by quickly reducing the pressure reactor and causing rapid decompression of citrus peel particles (e.g., heating to 160° C. and to 6 bar for 5 min and rapidly reducing the pressure to atmosphere). The pressure/heat treated particles are subjected to enzymatic hydrolysis, fermentation, or used as an adaptation medium for a microorganism.

### Example 8

Industrially useful molecules are extracted from orange peel in a series of steps. First, essential oils and fatty acids are extracted from orange peel by solvent extraction. Solid residues left from solvent extraction are either utilized for robust fermentation by an EMO, producing ethanol, or subjected to acid hydrolysis to produce pectin. Pectin is further processed to produce lower alkyl group molecules. Solid residues are also hydrolyzed to product hexoses, pentoses, or lignin, which are utilized as a feedstock for growing oil-producing microorganisms. An evolutionarily modified microorganism adapted to grow on lignin is utilized to convert lignin to oil.

### Example 9

Potato peels will be prepared to evolutionarily modify a micro-organism to better metabolize the potato peel's components. Potato peel is ground to pass a mesh screen. Alternatively, a blender or a cutting machine is used to cut down the potato peel into small particles. A steam and/or pressure are applied to break down the particles. To remove phenolic compound and other growth inhibitors, charcoal active carbon is used. pH of the extract is adjusted to neutral or close to neutral pH. pH-adjusted potato peel extract is mixed with a seed culture of bacterium to start fermentation. Alternatively, steam/pressure treated extract is incubated with 85% sulphuric acid to extract cellulose and hemicellulose fraction. The extracted cellulose and hemicellulose fraction, substantially free of lignin, are used to grow microorganisms adapted to robustly utilize cellulose and/or hemicellulose.

### Example 10

Sugar beet pulp (SBP) and sugar beet tail (SBT) will be prepared to evolutionarily modify a micro-organism to better metabolize the SBP or SBT components. SBP and SBT are collected as silages. SBP and SBT are minced to a particle size less than 0.3 cm. The minced silages are added to a bioreactor containing an inoculum containing an oil-producing microorganism adapted to grow on silage and yeast. Alternatively, minced silages are pretreated in autoclave for 15 min at 107° C. at 0.4-0.8 bar before adding to a bioreactor. An industrial scale flow digester is used as a bioreactor for continuous stirring, degasing and maintaining the temperature. The yeast fermented product, such as acetic acid, is extracted and converted to ethanol by esterification reaction and hydrogenation reaction. The oil-producing microorganism is separated from the culture and further process for oil extraction.

### Example 11

Raw biodiesel-derived waste glycerol is an excellent feedstock for the growth of heterotrophic microorganisms. However, this waste product contains contaminants (catalyst, methanol, free fatty acids, etc.) that can inhibit microbial growth.

Evolutionary Modification of an Algae (Chlorella protothecoides)

Chlorella protothecoides, a green algae, was evolutionarily modified to grow on biodiesel-derived waste glycerol in the presence of contaminants Chlorella protothecoidese can grow heterotrophically on refined glycerol and hyperaccumulates triglycerides (˜55% dry weight) that can be used to make biofuel. The Chlorella protothecoides strain CP25 was obtained from the UTEX algal culture collection. This strain was grown on minimal salt algae medium with 10 g/L pure glycerol as the carbon source in an Evolugator™ continuous culture apparatus. Slowly over time, the pure glycerol was incrementally replaced with waste glycerol until only waste glycerol was present in the medium. An evolutionary modified strain of Chlorella protothecoides (EVG09) was isolated from the Evolugator™ continuous culture apparatus that could grow robustly on waste glycerol.

Triglyceride Production and Isolation

A 25 L lab scale batch culture of the EVG09 strain was then grown on medium containing biodiesel-derived waste glycerol. The EVG09 strain was grown 7H9 algae medium (4.7 g/L) with Tween 80 (0.5 g/L) and 2 mL/L waste glycerol. The waste glycerol was heated at 90° C. for 10 min. to remove methanol and filter sterilized by passage through 0.22 μm filters prior to addition to the medium.

Triglycerides were harvested from the EVG09 cells, using heating or ultrasonication. Heating was determined to be the simplest method. By volumetric analysis, it was determined that the EVG09 cells retained the ability to hyperaccumulate triglycerides to ˜50% of the algal biomass. The EVG09 cells were subjected to fatty acid methyl ester analysis, which indicated that the EVG09 cells contained the following fatty acids.

TABLE 4
EVG09 cells-fatty acid profile

| Unsaturated Fatty Acids | Saturated Fatty Acids |
|---|---|
| 58.22% cis-oleate C18:1 w9c | 29.86% palmitate C16:0 |
| 1.93% cis-oleate dimethylacetal | 7.09% stearate C18:0 |
| 18:1 w9c DMA | |
| 1.33% cis-vaccenate 18:1 w5c | 0.33% myristate C14:0 |
| 0.80% cis-vaccenate dimethylacetal | |
| 18:1 w7c DMA | |
| 0.45% palmitoleate16:1 w7c | |

Biofuel Production

Biodiesel was produced by from a hexane extract of the EVG09 cells. Two hundred grams of cells were added to 40 mL of hexane, which was stirred for 1 hour at room temperature. The resulting mixture was added to a separation funnel for 1 hour and dried at 65° C. to remove residual hexane. Transesterification was achieved using base-catalyzed methodology. Two hundred mL of 99% methanol and 3.5 g/L NaOH was used to make a stock sodium methoxide solution. Titration was used to determine how much NaOH needed to account for the free fatty acids. It was determined that for the EVG09 algae oil it was negligible. Therefore the stock solution was used. 200 mL of methoxide was added to 1 L of pre-warmed oil at 55° C. The oil and methoxide we mixed slowly for 20 min. at 55° C. and allowed to settle for 12-24 hours and the glycerol layer was separated.

### Example 12

### Evolutionary Modification of an Algae (Chlorella protothecoides)

Chlorella protothecoides, a green algae, was evolutionarily modified to grow on Jatropha and castor bean seed cake. Chlorella protothecoidese can grow heterotrophically on refined glycerol and hyperaccumulates triglycerides (˜55% dry weight) that can be used to make biofuel.

The Chlorella protothecoides strain CP25 was obtained from the UTEX algal culture collection. This strain was grown on Bristol medium with 50 g/L carbohydrates (20% cellobiose, 4% raw glycerol, 2% glucose, mannose, galactose, and rhamnose; 9% xylose and arabinose) and 50 g/L Jatropha or castor bean seed cake hydrolysate.

Castor or Jatropha seeds were crushed in a hand crank expeller, producing 50 g of de-oiled seed cake. The 50 g of seed cake was then added to 250 mL of deionized water with 10 grams of NaOH pellets. The solution was mixed in a fume hood, covered and incubated at 65° C. overnight. The following morning the solution was removed from the incubator and autoclaved for 20 minutes at 15 psi and 121° C. (to neutralize the ricin). The seed cake hydrolyzate was cooled then diluted with the proper concentration of Bristol Medium to result in a 50 g/L dilution mix. After the medium was prepared, it was added into culture tubing by funnel, and autoclaved for 1 hour at 121° C./15 psi. After the tubing was cooled it was loaded into the Evolugator™ continuous culture apparatus and inoculated with Chlorella protothecoidese.

Evolutionary Modified Chlorella protothecoides with an Increased Growth Rate.

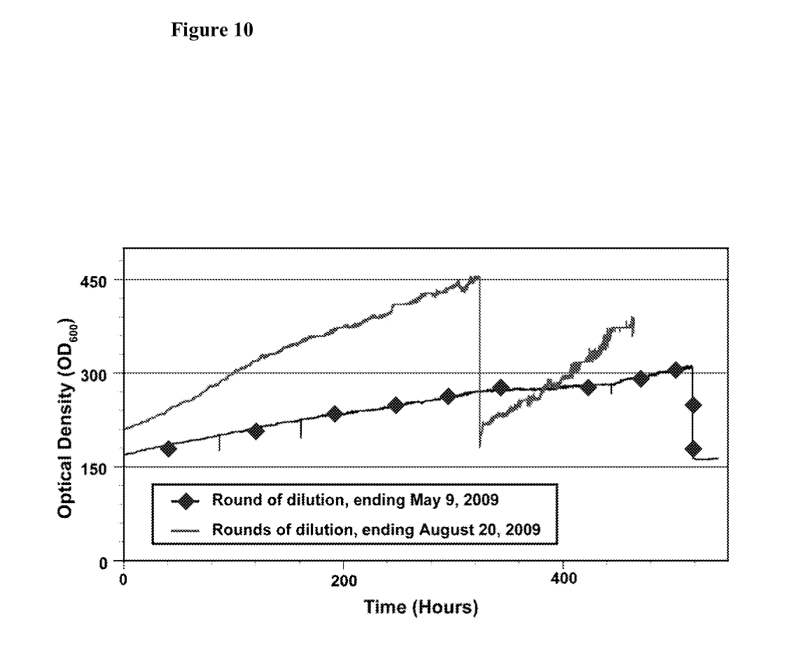

FIG. 10 illustrates the growth curves from samples obtained from the Evolugator™ showing C. protothecoides strain growing on a mix of carbohydrates, raw glycerol and Jatropha seed cake. The diamond line is the growth curve for the first dilution. The growth rate is slow (shallow slope) and the cells reach a maximum (relative) cells density of 300 arbitrary units. The non-diamond line is the growth curve for the same strain after 4 months of adaptation to the mixture. The growth rate is much faster (steep slope) and the cells reach a higher maximum density or 450 arbitrary units, indicating that the cells are converting more of the mixture into algal biomass.

### Example 13

Wild type Dunaliella Salina will be grown in a culture, with a carbonaceous material comprising refined glycerol and raw glycerol. The Dunaliella Salina is cultivated under conditions so that the amount of raw glycerol in the medium is slowly increased over a period of time so that the Dunaliella Salina is evolutionarily modified to utilize raw glycerol as the primary carbon source. This method produces an evolutionarily modified Dunaliella Salina.

The evolutionarily modified Dunaliella Salina will exhibit a higher maximum growth rate in a medium containing 100% raw glycerol than an unmodified strain of Dunaliella Salina cultivated in a medium containing 100% refined glycerol.

Next a culture of evolutionarily modified Dunaliella Salina will be grown in a culture medium comprising raw glycerol as the main carbon source. The Dunaliella Salina will then be isolated from the culture medium and optionally fractionated to obtain an extract comprising Dunaliella Salina lipids. The lipids will then be transesterified to obtain alkyl esters. The alkyl esters will then be separated and purified by filtration.

The alkyl esters will then be incorporated as a biodiesel in a distillate fuel oil composition comprising 50% fatty acids, and 50% diesel fuel by weight of the composition. The distillate fuel oil composition can be used to fuel a compression ignition internal combustion engine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. An evolutionarily modified organism wherein said evolutionarily modified organism's growth rate or end product yield during culture with a carbonaceous material is greater than the growth rate or end product yield of a non-evolutionarily modified organism, wherein said greater growth rate or product yield is due to a non-genetically engineered modification.

2. The organism of claim 1, wherein said organism is a bacterium, yeast, alga, or fungus.

3. The organism of claim 1, wherein said organism is Chlorella protothecoides.

4. The organism of claim 1, wherein said carbonaceous material is a stillage, bagasse, stover, peel, seed cake, seed, sugar beet, a wood chip, or any combination thereof.

5. The organism of claim 4, wherein said stillage is wet distillers grains with solubles (WDGS), dry distillers grains with solubles (DDGS), condensed distillers grains with solubles (CDS), or distillers grains (DG).

6. The organism of claim 1, wherein said organism has a greater end product yield of a non-evolutionarily modified organism.

7. The organism of claim 1, wherein said end product is a fatty acid.

8. An evolutionarily modified organism wherein said evolutionarily modified organism's growth rate or end product yield during culture with a carbonaceous material is greater than the growth rate or end product yield of a non-evolutionarily modified organism, wherein said evolutionarily modified organism is modified by continuous culture inside a flexible sterile tube filled with growth medium.

9. A method of producing an evolutionarily evolved organism comprising:
a) exposing an organism to a carbonaceous material;
b) continuously culturing said organism; and
c) producing an evolutionarily evolved organism from said organism, when said evolutionarily evolved organism has a growth rate or end product yield on said carbonaceous material that is greater than the growth rate or end product yield observed for said organism not continuously cultured with said carbonaceous material.

10. The method of claim 9, wherein said organism is a bacterium, yeast, alga, or fungus.

11. The method of claim 11, wherein said organism is Chlorella protothecoides.

12. The method of claim 9, wherein said carbonaceous material is a stillage, bagasse, stover, peel, seed cake, seed, sugar beet, a wood chip, or any combination thereof.

13. The method of claim 12, wherein said stillage is wet distillers grains with solubles (WDGS), dry distillers grains with solubles (DDGS), condensed distillers grains with solubles (CDS), or distillers grains (DG).

14. The method of claim 12, wherein said stillage is hydrolyzed stillage.

15. The method of claim 14, wherein said hydrolysis is enzymatic hydrolysis with alpha-amylase.

16. The method of claim 9, wherein said stillage is corn stillage.

17. The method of claim 9, wherein said organism is placed in a flexible tubing, wherein said tubing is subdivided by an operation of a gate into one or more discreet chambers.

18. The method of claim 9, wherein said organism is a naturally occurring organism.

19. The method of claim 9, wherein said organism is a genetically modified organism.

20. A method of producing an end product comprising:
a) contacting an evolutionarily evolved organism with a carbonaceous material
b) culturing said carbonaceous material with said organism; and
c) separating an end product from said culture of step b).

21. The method of claim 20, wherein said organism is a bacterium, yeast, alga, or fungus.

22. The method of claim 20, wherein said organism is Chlorella protothecoides.

23. The method of claim 20, wherein said carbonaceous material is a stillage, bagasse, stover, peel, seed cake, seed, sugar beet, a wood chip, or any combination thereof.

24. The method of claim 23, wherein said stillage is wet distillers grains with solubles (WDGS), dry distillers grains with solubles (DDGS), condensed distillers grains with solubles (CDS), or distillers grains (DG).

25. The method of claim 23, wherein said stillage is hydrolyzed stillage.

26. The method of claim 25, wherein said hydrolysis is enzymatic hydrolysis with alpha-amylase.

27. The method of claim 20, wherein said stillage is corn stillage.

28. The method of claim 20, further comprising:
d) extracting oil from said end product.

29. The method of claim 28, further comprising:
e) processing said oil to a biofuel.

30. The method of claim 29, wherein said biofuel is a biodiesel.

31. The method of claim 20, wherein said end product is a protein.

32. The method of claim 31, wherein said protein is an algal protein

33. The method of claim 20, wherein said end product is a vitamin.

34. A biofuel manufacturing plant, comprising:
a) a culture unit comprising an evolutionary modified organism and a carbonaceous material wherein said carbonaceous material is said evolutionary modified organism's primary carbon source;
b) a processing unit for processing the organism grown in said culture unit to obtain a lipid fraction;
and a
c) a reactor unit for transesterifying a first lipid fraction with an alcohol to obtain alkyl esters;
d) a separator unit to separate the alkyl esters from by-products.

35. The plant according to claim 34, further comprising a protein recovery unit for recovering protein from said organism.

36. The plant according to claim 34, wherein the culture unit has a growth chamber for growing the organism in a heterotrophic environment.

37. The plant according to claim 34, further comprising a filter unit for recovering and filtering the alkyl esters to obtain a biofuel product.

38. The plant according to claim 34, wherein said carbonaceous material is a stillage, bagasse, stover, peel, seed cake, seed, sugar beet, a wood chip, or any combination thereof.

39. A method for producing a biofuel product from a by-product of biofuel production, comprising the steps of: obtaining a by-product of biofuel production, said by-product of biodiesel production comprising an agricultural product; growing a evolutionary modified organism with said byproduct of bio fuel production, and wherein the agricultural product is the primary carbon source; isolating and recovering the evolutionary modified organism from said growing step; fractionating the evolutionary modified organism from said growing step to obtain a first lipid fraction; transesterifying the first lipid fraction with an alcohol to obtain alkyl esters as the biofuel product; and recovering the biofuel product.

40. The method of claim 39, wherein said organism is a bacterium, yeast, alga, or fungus.

41. The method of claim 39, wherein said organism is Chlorella protothecoides.

42. The method of claim 39, wherein said agricultural product is a glycerol, stillage, bagasse, stover, peel, seed cake, seed, sugar beet, a wood chip, or any combination thereof.

43. The method of claim 39, wherein said biofuel is a biodiesel.
